(19) United States
(12) Patent Application Publication
(10) Pub. No.: US 2024/0317875 A1
(43) Pub. Date: Sep. 26, 2024

(54) USE OF ANTI-CD40 ANTIBODIES FOR TREATMENT OF INFLAMMATORY CONDITIONS

(71) Applicant: Boehringer Ingelheim International GmbH, Ingelheim am Rhein (DE)

(72) Inventors: Fenglei HUANG, Princeton, NJ (US); Meera RAMANUJAM, Danbury, CT (US); Juergen Theodor STEFFGEN, Ulm (DE); Yasuhiro TSUDA, Kobe (JP); Sudha VISVANATHAN, Hartsdale, NY (US)

(73) Assignee: Boehringer Ingelheim International GmbH, Ingelheim am Rhein (DE)

(21) Appl. No.: 18/588,587

(22) Filed: Feb. 27, 2024

Related U.S. Application Data
(63) Continuation of application No. 17/477,905, filed on Sep. 17, 2021, now abandoned.
(60) Provisional application No. 63/080,896, filed on Sep. 21, 2020.
(60) Provisional application No. 63/138,014, filed on Jan. 15, 2021.
(60) Provisional application No. 63/193,776, filed on May 27, 2021.

(51) Int. Cl. C07K 16/28; A61K 39/00; A61P 37/02

(52) U.S. Cl. CPC C07K 16/2878; A61P 37/02; A61K 2039/505; C07K 2317/565

(57) ABSTRACT

The present invention relates to anti-CD40 antibodies and therapeutic methods for using the same for treating and/or preventing autoimmune or inflammatory diseases.

### CROSS-REFERENCE TO RELATED APPLICATIONS

The present application is a Continuation of U.S. application Ser. No. 17/477,905, filed Sep. 17, 2021, which claims priority to U.S. Provisional Application Nos. 63/193,776, filed May 27, 2021, 63/138,014, filed Jan. 15,2021, and 63/080,896, filed Sep. 21, 2020, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Sep. 14, 2021, is named 09-0706-US-4-2021-09-17-Sequence-Listing.txt and is 106,888 bytes in size.

### FIELD OF THE INVENTION

This invention generally relates to the use of humanized anti-CD40 antibodies for the treatment and/or prevention of various autoimmune or inflammatory diseases, more particularly for the treatment of various diseases or disorders characterized by cells expressing CD40 are disclosed.

### BACKGROUND OF THE INVENTION

CD40 is a 48 kDa type I integral membrane glycoprotein and a member of the tumor necrosis factor (TNF) receptor superfamily. CD40 is expressed on a variety of cell types including normal and neoplastic B cells, interdigitating cells, carcinomas, epithelial cells (e.g. keratinocytes), fibroblasts (e.g. synoviocytes) and platelets. It is also present on monocytes, macrophages, some endothelial cells, and follicular dendritic cells. CD40 is expressed early in B cell ontogeny, appearing on B cell precursors subsequent to the appearance of CD10 and CD19, but prior to expression of CD21, CD23, CD24, and appearance of surface immunoglobulin M (slgM). CD40 has also been detected on tonsil and bone marrow-derived plasma cells.

The ligand of CD40 is CD40L (also referred to as CD154, gp39, and TRAP), a TNF superfamily member. CD40L is a transmembrane protein expressed predominantly on activated CD4⁺ T cells and a small subset of CD8+ T cells.

The interaction of CD40 with CD40L induces both humoral and cell-mediated immune responses. CD40 regulates this ligand-receptor pair to activate B cells and other antigen-presenting cells (APC) including dendritic cells (DCs). The function of CD40 on B cells has been studied extensively. Activation of CD40 on B cells induces proliferation, differentiation into antibody secreting cells and isotype switching in germinal centers of secondary lymphoid organs. In vitro studies have shown direct effects of CD40 activation on cytokine production (IL-6, IL-10, TNF-α, LT-α), expression of adhesion molecules and costimulatory receptors (ICAM, CD23, CD80 and CD86), and increased expression of MHC class I, MHC class II, and TAP transporter by B lymphocytes. For most of these processes, CD40 acts in concert with either cytokines or other receptor-ligand interactions.

CD40 signaling on monocytes and DCs results in enhanced survival as well as secretion of cytokines (IL-1, IL-6, IL-8, IL-10, IL-12, TNF-α and MIP-1a). CD40 ligation on these APCs also leads to the up-regulation of costimulatory molecules such as (ICAM-1, LFA-3, CD80, and CD86). Activation of CD40 receptors is one of the critical signals that allow the full maturation of DC into efficient APCs driving T cell activation.

Recent studies in mouse models showed that CD40 signaling on dendritic cells also plays an important role in the generation of TH17 cells which are considered as mediators of autoimmunity in diseases such as arthritis and multiple sclerosis.

The availability of CD40 and CD40L knock-out mice as well as agonistic and antagonistic anti-mouse antibodies offered the possibility to study the role of CD40-CD40L interactions in several disease models. Administration of blocking anti-CD40L has been demonstrated to be beneficial in several models of autoimmunity including spontaneous diseases like lupus nephritis in SNF1 mice or diabetes in NOD mice or in experimentally induced forms of disease like collagen-induced arthritis (CIA) or experimental autoimmune encephalomyelitis (EAE). CIA in mice was inhibited by an anti-CD40L mAb which blocked the development of joint inflammation, serum antibody titers to collagen, the infiltration of inflammatory cells into the subsynovial tissue in addition to the erosion of cartilage and bone. Both for lupus nephritis and EAE, it was demonstrated that anti-CD40L could also alleviate ongoing disease, confirming the role of CD40-CD40L in the effector phase of the disease.

The role for CD40-CD40L interactions in the development of EAE was also studied in CD40L-deficient mice that carried a transgenic T cell receptor specific for myelin basic protein. These mice failed to develop EAE after priming with antigen, and CD4+ T cells remained quiescent and produced no INF-γ.

Furthermore, inhibitory antibodies directed against CD40 showed beneficial effects in inflammatory disease models such as EAE. Lamann and colleagues demonstrated that the antagonistic mouse anti-human CD40 mAb mu5D12 and a chimeric version of this mAb effectively prevented clinical expression of chronic demyelinating EAE in outbred marmoset monkeys. A follow-up study showed that therapeutic treatment with the chimeric anti-human CD40 antibody reduces MRI-detectable inflammation and delays enlargement of pre-existing brain lesions in the marmoset EAE model.

Anti-CD40 antibodies with agonistic activity were tested in mouse models of arthritis with some conflicting results. As expected for an immunostimulatory agent, the agonistic anti-mouse CD40 mAb FGK45 was shown to exacerbate disease in the DBA/1 mouse model of CIA. However, in another chronic CIA model FGK45, and another agonistic anti-mouse CD40 mAb, 3/23, both exhibited positive therapeutic effects. It was postulated by this group that the agonistic antibodies in this therapeutic treatment regimen have a beneficial effect by inducing immune deviation towards a Th2 response with decreased levels of IFN-γ and increased levels of IL-4 and IL-10.

The prevention of transplant rejection by blocking CD40/CD154 interactions has also been documented. The use of ch5D12, a chimeric anti-CD40 antagonist, in renal allograft studies in rhesus monkeys indicates that antagonism of CD40 is sufficient for disease modification and lengthening mean survival times past 100 days. When ch5D12 was combined with an anti-CD86 antibody and given only at the initiation of the allograft studies followed by prolonged treatment with cyclosporine, mean survival times greater than 4 years were achieved, indicating this combination can potentially induce tolerance.

Thus, preclinical studies that provide evidence for the crucial role of the CD40-CD40L dyad in driving an efficient T cell-dependent immune response. Blocking of CD40 signaling is therefore recognized as a suitable and needed therapeutic strategy to suppress a pathogenic autoimmune response in diseases such as RA, lupus nephritis multiple sclerosis or psoriasis. However, to date, there are no CD40 antibodies that have been approved for therapeutic intervention of such disorders due to the findings that anti-CD40 antibodies previously in development were shown to have significant side effects.

For example, all treatments available for lupus nephritis may be associated with significant toxicity (for example, infertility, infection, malignancy). Furthermore the complete response rates remain low and within the responders there is a high rate of relapses justifying long-term maintenance therapy. Recently conducted Phase III trials in lupus nephritis (e.g. rituximab, abatacept) have failed to meet their primary endpoints. Taken together, there is a high unmet need for new therapies in lupus nephritis. This need could be addressed by the humanized anti-CD40 antibodies described herein and in US20110243932 that specifically bind CD40 and which show the antigen binding specificity, affinity, and pharmacokinetic and pharmacodynamic properties that allow use thereof in therapeutic intervention of CD40 based disorders such as lupus nephritis.

### BRIEF SUMMARY OF THE INVENTION

In an embodiment, the present invention relates to a method of treating or preventing an autoimmune or inflammatory disease in a subject in need thereof, the method comprising administering to the subject a composition comprising an anti-CD40 (anti-cluster of differentiation 40) antibody,

wherein the anti-CD40 antibody comprises a heavy chain and a light chain, wherein the heavy chain sequence and light chain sequence are selected from the group consisting of: a) a heavy chain CDR1 sequence selected from the group consisting of SEQ ID NO: 9 through SEQ ID NO:11, a heavy chain CDR2 sequence selected from the group consisting of SEQ ID NO:12 through SEQ ID NO:15, and a heavy chain CDR3 sequence selected from the group consisting of SEQ ID NO:16 through SEQ ID NO:17; and b) the light chain CDR1 sequence has a sequence selected from the group consisting of SEQ ID NO:18 through SEQ ID NO:21, a light chain CDR2 sequence of SEQ ID NO:22 through SEQ ID NO:23, and a light chain CDR3 sequence selected from the group consisting of SEQ ID NO:24 through SEQ ID NO:25; or
wherein the anti-CD40 antibody comprises a variable heavy chain domain comprising any of SEQ ID NO:42, SEQ ID NO:44, SEQ ID NO:46, SEQ ID NO:48, SEQ ID NO:53, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73; and a variable light chain domain comprising any of SEQ ID NO:41, SEQ ID NO:43, SEQ ID NO:45, SEQ ID NO:47, SEQ ID NO:49, SEQ ID NO:50, SEQ ID NO:51, SEQ ID NO:52, SEQ ID NO:54, SEQ ID NO:55, SEQ ID NO:56, SEQ ID NO:74, SEQ ID NO:75, or SEQ ID NO:76; or
wherein the anti-CD40 antibody comprises a heavy chain sequence and a light chain sequence comprising the amino acid sequences of SEQ ID NO:27 and SEQ ID NO:26, respectively; SEQ ID NO:28 and SEQ ID NO:26, respectively; SEQ ID NO:29 and SEQ ID NO:26, respectively; SEQ ID NO:30 and SEQ ID NO:26, respectively; SEQ ID NO:32 and SEQ ID NO:31, respectively; SEQ ID NO:33 and SEQ ID NO:31, respectively; SEQ ID NO:34 and SEQ ID NO:31, respectively; SEQ ID NO:35 and SEQ ID NO:31, respectively; SEQ ID NO:37 and SEQ ID NO:36, respectively; SEQ ID NO:38 and SEQ ID NO:36, respectively; SEQ ID NO:39 and SEQ ID NO:36, respectively; or SEQ ID NO:40 and SEQ ID NO: 36, respectively;
wherein the composition comprises 80 mg, 120 mg, 180 mg or 240 mg of the anti-CD40 antibody;
wherein a single-dose administration of the composition comprising 80 mg of the anti-CD40 antibody results in Cmax (ng mL-1) of about 888 to about 1550, AUC0-tz (μg·h mL−1) of about 126 to about 365, or AUC0-inf (μg·h mL-1) of about 330 to about 464; or wherein a single-dose administration of the composition comprising 120 mg of the anti-CD40 antibody results in Cmax (ng mL−1) of about 5160 to about 7210, AUC0-tz (μg·h mL−1) of about 1110 to about 2010, or AUC0-inf (μg·h mL−1) of about 1120 to about 2020; or
wherein a single-dose administration of the composition comprising 180 mg of the anti-CD40 antibody results in Cmax (ng mL−1) of about 8650 to about 16300, AUC0-tz (μg·h mL−1) of about 2900 to about 6380, or AUC0-inf (μg·h mL−1) of about 2020 to about 2910; or
wherein a single-dose administration of the composition comprising 240 mg of the anti-CD40 antibody results in Cmax (ng mL−1) of about 15700 to about 21300, AUC0-tz (μg·h mL−1) of about 5680 to about 7750, or AUC0-inf (μg·h mL−1) of about 5610 to about 7780; or
wherein a multiple-dose administration (q1w or once every week) of the composition comprising 240 mg of the anti-CD40 antibody results in Cmax,1 (μg mL−1) of about 23 after the first dose or AUC_{T}, 1 (μg·h mL−1) of about 2600 after the first dose; or
wherein a multiple-dose administration (q1w or once every week) of the composition comprising 240 mg of the anti-CD40 antibody results in Cmax,4 (μg mL−1) of about 74 after the frouth dose, or Cmin, 4(μg mL−1) of about 49 after the fourth dose, or AUC_{T}, 4 (μg·h mL−1) of about 10900 after the fourth dose.

The method according to the above embodiment, wherein the autoimmune or inflammatory disease is selected from the group consisting of lupus nephritis, rheumatoid arthritis, multiple sclerosis, proliferative lupus glomerulonephritis, inflammatory bowel disease (IBD), psoriasis, idiopathic thrombocytopenic purpura (ITP), Crohn's Disease and systemic lupus erythematosus (SLE), Hashimoto's thyroiditis, primary myxoedema, thyrotoxicosis/Graves disease, pernicious anaemia, autoimmune atrophic gastritis, autoimmune carditis, Addison's disease, premature menopause, type 1-diabetes mellitus, Good pasture's syndrome, myasthenia gravis, autoimmune haemolytic anaemia, idiopathic leucopenia, primary biliary cirrhosis, active chronic hepatitis (HBs Ag negative), cryptogenic cirrhosis, Sjogren's syndrome, dermatomyositis, scleroderma, mixed tissues connective disease, discoid lupus erythematosus, and systemic vasculitis.

The method according to the above embodiment, wherein the antibody comprises a heavy chain CDR1 sequence of SEQ ID NO: 10, a heavy chain CDR2 sequence of SEQ ID NO:13 and a heavy chain CDR3 sequence of SEQ ID NO:16; and wherein said antibody comprises a light chain CDR1 sequence of SEQ ID NO:19, a light chain CDR2 sequence of SEQ ID NO:22 and a light chain CDR3 sequence of SEQ ID NO:24.

The method according to the above embodiment, wherein said antibody comprises a heavy chain CDR1 sequence of SEQ ID NO: 9, a heavy chain CDR2 sequence of SEQ ID NO:14 and a heavy chain CDR3 sequence of SEQ ID NO:16; and wherein said antibody comprises a light chain CDR1 sequence of SEQ ID NO:20, a light chain CDR2 sequence of SEQ ID NO:22 and a light chain CDR3 sequence of SEQ ID NO:24.

The method according to the above embodiment, wherein said antibody comprises a heavy chain CDR1 sequence of SEQ ID NO: 11, a heavy chain CDR2 sequence of SEQ ID NO:15 and a heavy chain CDR3 sequence of SEQ ID NO:17; and wherein said antibody comprises a light chain CDR1 sequence of SEQ ID NO:21, a light chain CDR2 sequence of SEQ ID NO:23 and a light chain CDR3 sequence of SEQ ID NO:25.

The method according to the above embodiment, wherein the antibody comprises a heavy chain variable domain and a light chain variable region comprising the amino acid sequences of SEQ ID NO:27 and SEQ ID NO:26, respectively; SEQ ID NO:28 and SEQ ID NO:26, respectively; SEQ ID NO:29 and SEQ ID NO:26, respectively; SEQ ID NO:30 and SEQ ID NO:26, respectively; SEQ ID NO:32 and SEQ ID NO:31, respectively; SEQ ID NO:33 and SEQ ID NO:31, respectively; SEQ ID NO:34 and SEQ ID NO:31, respectively; SEQ ID NO:35 and SEQ ID NO:31, respectively; SEQ ID NO:37 and SEQ ID NO:36, respectively; SEQ ID NO:38 and SEQ ID NO:36, respectively; SEQ ID NO:39 and SEQ ID NO:36, respectively; SEQ ID NO:40 and SEQ ID NO: 36, respectively.

The method according to the above embodiment, wherein the antibody comprises: a heavy chain variable domain comprising SEQ ID NO:44 and a light chain variable domain comprising SEQ ID NO:43; or a heavy chain variable domain comprising SEQ ID NO:53 and a light chain variable domain comprising SEQ ID NO:52; or a heavy chain variable domain comprising SEQ ID NO:58 and a light chain variable domain comprising SEQ ID NO:56.

The method according to the above embodiment, wherein the antibody comprises: a heavy chain sequence comprising SEQ ID NO:30 and a light chain sequence comprising SEQ ID NO:26; or a heavy chain sequence comprising SEQ ID NO:35 and a light chain sequence comprising SEQ ID NO:31; or a heavy chain sequence comprising SEQ ID NO:40 and a light chain sequence comprising SEQ ID NO:36.

The method according to the above embodiment, wherein the autoimmune or inflammatory disease is selected from the group consisting of lupus nephritis, graft v. host disease, autoimmune or inflammatory disease, and CD40-related disorder.

In another embodiment, the invention relates to a method of treating or preventing an autoimmune or inflammatory disease in a subject in need thereof, the method comprising administering to the subject a composition comprising an anti-CD40 (anti-cluster of differentiation 40) antibody,

wherein the anti-CD40 antibody comprises a heavy chain and a light chain, wherein the heavy chain sequence and light chain sequence are selected from the group consisting of: a) a heavy chain CDR1 sequence selected from the group consisting of SEQ ID NO: 9 through SEQ ID NO:11, a heavy chain CDR2 sequence selected from the group consisting of SEQ ID NO:12 through SEQ ID NO:15, and a heavy chain CDR3 sequence selected from the group consisting of SEQ ID NO:16 through SEQ ID NO:17; and b) the light chain CDR1 sequence has a sequence selected from the group consisting of SEQ ID NO:18 through SEQ ID NO:21, a light chain CDR2 sequence of SEQ ID NO:22 through SEQ ID NO:23, and a light chain CDR3 sequence selected from the group consisting of SEQ ID NO:24 through SEQ ID NO:25; or
wherein the anti-CD40 comprises a variable heavy chain domain comprising any of SEQ ID NO:42, SEQ ID NO:44, SEQ ID NO:46, SEQ ID NO:48, SEQ ID NO:53, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73; and a variable light chain domain comprising any of SEQ ID NO:41, SEQ ID NO:43, SEQ ID NO:45, SEQ ID NO:47, SEQ ID NO:49, SEQ ID NO:50, SEQ ID NO:51, SEQ ID NO:52, SEQ ID NO:54, SEQ ID NO:55, SEQ ID NO:56, SEQ ID NO:74, SEQ ID NO:75, or SEQ ID NO:76; or
wherein the anti-CD40 antibody comprises a heavy chain sequence and a light chain sequence comprising the amino acid sequences of SEQ ID NO:27 and SEQ ID NO:26, respectively; SEQ ID NO:28 and SEQ ID NO:26, respectively; SEQ ID NO:29 and SEQ ID NO:26, respectively; SEQ ID NO:30 and SEQ ID NO:26, respectively; SEQ ID NO:32 and SEQ ID NO:31, respectively; SEQ ID NO:33 and SEQ ID NO:31, respectively; SEQ ID NO:34 and SEQ ID NO:31, respectively; SEQ ID NO:35 and SEQ ID NO:31, respectively; SEQ ID NO:37 and SEQ ID NO:36, respectively; SEQ ID NO:38 and SEQ ID NO:36, respectively; SEQ ID NO:39 and SEQ ID NO:36, respectively; or SEQ ID NO:40 and SEQ ID NO: 36, respectively;
wherein the composition comprises 120 mg or 180 mg of the anti-CD40 antibody, and wherein the administration results in improvement in total SLEDAI-SELENA score and its subscores in the subject as compared to placebo.

In an embodiment relating to any of the aspects or embodiments, the administration results in an improvement in total SLEDAI or non-renal SLEDAI scores in the subject as compared to placebo. In some embodiments, the improvement is ≥5% at Weeks 26 or 52. In some embodiments, the improvement is ≥10% at Weeks 26 or 52.

In another embodiment, the invention relates to a method of determining the treatment efficacy of an anti-CD40 antibody in treating or preventing an autoimmune or inflammatory disease in a subject, the method comprising administering to the subject a composition comprising the anti-CD40 antibody, measuring the levels of an activated B-cell subset in the subject, wherein a decrease in the levels of the activated B-cell subset (when comparing the levels before and after the treatment) is indicative of efficacy. In a related embodiment, the activated B-cell subset is selected from the group consisting of CD19⁺IgD⁻CD27⁻CD95⁺, CD19+IgD+CD27⁻CD95⁺, CD19⁺IgD⁻CD27⁺CD95⁺ and CD19⁺IgD⁺CD27⁺CD95⁺. In a related embodiment, the anti-CD40 antibody is any of those disclosed herein. In a related embodiment, the autoimmune or inflammatory disease is any of the ones disclosed herein.

In another embodiment, the invention relates to a method of decreasing the levels of an activated B-cell subset in a subject suffering from an autoimmune or inflammatory disease, the method comprising administering to the subject a composition comprising an anti-CD40 antibody, wherein the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺. In a related embodiment, the anti-CD40 antibody is any of the ones disclosed herein. In a related embodiment, the autoimmune or inflammatory disease is any of the ones disclosed herein.

In another embodiment, the invention relates to a method of treating or preventing an autoimmune or inflammatory disease in a subject in need thereof, the method comprising administering to the subject a composition comprising 80 mg, 120 mg, 180 mg or 240 mg of an anti-CD40 (anti-cluster of differentiation 40) antibody, wherein the subject exhibits (or has been determined to exhibit) the presence of an activated B-cell subset. In a related embodiment, the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺. In a related embodiment, the anti-CD40 antibody is any of those disclosed herein. In a related embodiment, the autoimmune or inflammatory disease is any of the ones disclosed herein.

In another embodiment, the invention relates to a method of treating or preventing an autoimmune or inflammatory disease in a subject in need thereof, the method comprising (a) determining that the subject exhibits the presence of an activated B-cell subset (e.g., by testing a biological sample obtained from the subject), (b) administering to the subject a composition comprising 80 mg, 120 mg, 180 mg or 240 mg of an anti-CD40 (anti-cluster of differentiation 40) antibody. In a related embodiment, the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺. In a related embodiment, the anti-CD40 antibody is any of those disclosed herein. In a related embodiment, the autoimmune or inflammatory disease is any of the ones disclosed herein.

In another embodiment, the invention relates to a method of treating or preventing an autoimmune or inflammatory disease in a subject in need thereof, the method comprising administering to the subject a composition comprising 80 mg, 120 mg, 180 mg or 240 mg of an anti-CD40 (anti-cluster of differentiation 40) antibody, wherein the subject has been determined to exhibit the presence of an activated B-cell subset. In a related embodiment, the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺. In a related embodiment, the anti-CD40 antibody is any of those disclosed herein. In a related embodiment, the autoimmune or inflammatory disease is any of the ones disclosed herein.

It will be understood that any of the herein disclosed methods, administration schemes and/or dosing regimens, in particular the methods of treating or preventing an autoimmune or inflammatory disease in a subject in need thereof, also equally apply to the use of any of the disclosed anti-CD40 (anti-cluster of differentiation 40) antibodies in such methods, administration schemes and/or dosing regimens: i.e. an anti-CD40 antibody, as disclosed herein, for use in the treatment, prevention, reducing and/or amelioration of any of the disclosed diseases and/or conditions. In other words, the invention also provides for the use of an anti-CD40 antibody, as disclosed herein, for the manufacture of a medicament for the treatment, prevention, reducing and/or amelioration of any of the disclosed diseases and/or conditions.

### BRIEF DESCRIPTION OF SEVERAL VIEWS OF THE DRAWINGS

The accompanying drawings, which are included to provide further understanding of the present invention and are incorporated in and constitute a part of this specification, illustrate aspects of the subject technology and together with the description serve to explain the principles of the present invention.

FIG. 1 shows the mean (±SD) BI 655064 plasma concentration-time profile following single-dose administration to Chinese and Japanese subjects in study 1 of Example 2. s.c., subcutaneous; SD, standard deviation. Upper panels: linear scaler with SD. Lower panels: semi-log scale.

FIG. 2 shows the mean (±SD) BI 655064 plasma concentration-time profile following multiple-dose administration to Chinese subjects in study 2 of Example 2. SD, standard deviation. Upper panel: linear scaler with SD. Lower panel: semi-log scale.

FIG. 3 shows the mean (±SD) inhibition of CD40 receptor occupancy over time following single-dose administration to Chinese and Japanese subjects in study 1 and following multiple dose administration to Chinese subjects in study 2 of Example 2. SD, standard deviation. Note: For Study 2, a drop in the unstained raw data values was observed. The calculation of the inhibition of CD40 receptor occupancy is based on ratios of fluorescence values from stained vs unstained samples. Therefore, small deviations in staining intensity of the unstained samples can result in large effects on the calculated percent inhibition results in certain instances.

FIG. 4 shows the relationship between BI 655064 plasma concentrations and inhibition of CD40 receptor occupancy following single-dose administration to Chinese and Japanese subjects in study 1 of Example 2. Dotted line indicates 90% inhibition.

FIG. 5 shows the study design described in Example 3. Abbreviations are as follows: MMF, mycophenolate mofetil; SLEDAI, Systemic Lupus Erythematosus Disease Activity Index; PRR, Partial Renal Response; SoC, standard of care.

FIGS. 6A-6D show the efficacy of BI 655064 based on UP from 24-hour collections. FIG. 6A shows the proportion of patients achieving a CRR at Week 26. FIG. 6B shows the proportion of patients achieving a CRR or PRR at Week 26. FIG. 6C shows the adjusted proportion of patients achieving a CRR at Week 52. FIG. 6D shows the proportion of patients achieving a CRR or PRR at Week 52. CRR, complete renal response; PRR, partial renal response; UP, urine protein.

FIGS. 7A-7B show the efficacy of BI 655064 based on UP/UC from spot urine. FIG. 7A. Proportion of patients achieving CRR at Week 52. FIG. 7B. Adjusted proportion of patients achieving cCRR at Weeks 46 and 52. *Post hoc analysis showing modelled proportion of patients achieving CRR at Weeks 46 and 52; In the multiple comparison procedures and modelling (MCPMod) analysis, two significant models (p<0.2) were selected, sigEmax and exponential. CRR, complete renal response; cCRR, confirmed complete renal response; PRR, partial renal response; UP/UC, urine protein/urine creatinine ratio.

FIGS. 8A-8D show the assessment of further endpoints. FIG. 8A. Time to CRR. FIG. 8B. Median change in UP/UC based on spot urine from baseline over time. FIG. 8C. Mean change from baseline in total SLEDAI at Weeks 26 and 52. FIG. 8D. Mean change from baseline in non-renal SLEDAI at Weeks 26 and 52. SLEDAI, systemic lupus erythematosus disease activity index.

FIGS. 9A-9B show the change from baseline in CD95+memory B-cell subsets. FIG. 9A. At Week 12. FIG. 9B. At Week 26. *p<0.05 BI 240 mg vs placebo; **p<0.05 BI 240 mg and 180 mg vs placebo.

### DETAILED DESCRIPTION OF THE INVENTION

CD40 mediated signalling is now recognized as being involved in a variety of target disorders. Despite the availability of a variety of preclinical data showing that intervention in these disorders would be therapeutically beneficial, there remains a need for antagonistic anti-CD40 antibodies that can be used in the treatment of autoimmune diseases such as lupus nephritis.

The terms “CD40” and “CD40 surface antigen” refer to an approximately 48 kD glycoprotein expressed on the surface of normal and neoplastic B cells, which acts as a receptor for signals involved in cellular proliferation and differentiation (Ledbetter et al., 1987, J. Immunol. 138:788-785). A cDNA molecule encoding CD40 has been isolated from a library prepared from the Burkitt lymphoma cell line Raji (Stamenkovic et al., 1989, EMBO J. 8:1403).

A used herein, a cell that endogenously expresses CD40 is any cell characterized by the surface expression of CD40, including, but not limited to, normal and neoplastic B cells, interdigitating cells, basal epithelial cells, carcinoma cells, macrophages, endothelial cells, follicular dendritic cells, tonsil cells, and bone marrow-derived plasma cells. In some embodiments, the CD40 molecule is a human CD40 molecule.

The antibodies of the invention specifically bind to human recombinant and native CD40. A humanized monoclonal antibody wherein said antibody specifically binds to human CD40 having an antagonistic activity IC50 of less than 1 nM and has no agonism up to 100 μg/ml in B cell proliferation and wherein said antibody is further characterized in that the antibody has an in vivo half-life in non-human primates that is at least 10 days.

The generalized structure of antibodies or immunoglobulin is well known to those of skill in the art, these molecules are heterotetrameric glycoproteins, typically of about 150,000 daltons, composed of two identical light (L) chains and two identical heavy (H) chains. Each light chain is covalently linked to a heavy chain by one disulfide bond to form a heterodimer, and the heterotrameric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (V_{H}), followed by three or four constant domains (C_{H1}, C_{H2}, C_{H3}, and C_{H4}), as well as a hinge region between C_{H1} and C_{H2}. Each light chain has two domains, an amino-terminal variable domain (V_{L}) and a carboxy-terminal constant domain (C_{L}). The V_{L} domain associates non-covalently with the V_{H} domain, whereas the C_{L} domain is commonly covalently linked to the C_{H1} domain via a disulfide bond. Particular amino acid residues are believed to form an interface between the light and heavy chain variable domains (Chothia et al., 1985, J. Mol. Biol. 186:651-663.)

Certain domains within the variable domains differ extensively between different antibodies i.e., are “hypervariable.” These hypervariable domains contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, In: Sequences of Proteins of Immunological Interest, 5^{th} Ed. Public Health Service, National Institutes of Health, Bethesda, Md., whereas HVLs are structurally defined according to the three-dimensional structure of the variable domain, as described by Chothia and Lesk, 1987, J. Mol. Biol. 196: 901-917. Where these two methods result in slightly different identifications of a CDR, the structural definition is preferred. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. The CDR1, CDR2, CDR3 of the heavy and light chains therefore define the unique and functional properties specific for a given antibody.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely β-sheet configuration of the FRs brings the CDRs within each of the chains into close proximity to each other as well as to the CDRs from the other chain. The resulting conformation contributes to the antigen binding site (see Kabat et al., 1991, NIH Publ. No. 91-3242, Vol. I, pages 647-669), although not all CDR residues are necessarily directly involved in antigen binding.

FR residues and Ig constant domains are not directly involved in antigen binding, but contribute to antigen binding and/or mediate antibody effector function. Some FR residues are thought to have a significant effect on antigen binding in at least three ways: by noncovalently binding directly to an epitope, by interacting with one or more CDR residues, and by affecting the interface between the heavy and light chains. The constant domains are not directly involved in antigen binding but mediate various Ig effector functions, such as participation of the antibody in antibody dependent cellular cytotoxicity (ADCC), complement dependent cytotoxicity (CDC) and antibody dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. IgG and IgA are further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of the classes of native immunoglobulins are well known.

The terms, “antibody”, “anti-CD40 antibody”, “humanized anti-CD40 antibody”, and “variant humanized anti-CD40 antibody” are used herein in the broadest sense and specifically encompass monoclonal antibodies (including full length monoclonal antibodies), polyclonal antibodies, multispecific antibodies (e.g., bispecific antibodies), and antibody fragments such as variable domains and other portions of antibodies that exhibit a desired biological activity, e.g., CD40 binding.

The term “monoclonal antibody” (mAb) refers to an antibody of a population of substantially homogeneous antibodies; that is, the individual antibodies in that population are identical except for naturally occurring mutations that may be present in minor amounts. Monoclonal antibodies are highly specific, being directed against a single antigenic determinant, an “epitope”. Therefore, the modifier “monoclonal” is indicative of a substantially homogeneous population of antibodies directed to the identical epitope and is not to be construed as requiring production of the antibody by any particular method. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352: 624-628, and Marks et al., 1991, J. Mol. Biol. 222: 581-597.

A humanized anti-CD40 antibody can be selected from any class of immunoglobulins, including IgM, IgG, IgD, IgA and IgE, and any isotype, including IgG₁, IgG₂, IgG₃, IgG₄, IgA₁ and IgA₂. For example, the constant domain can be a complement fixing constant domain where it is desired that the humanized antibody exhibit cytotoxic activity, and the isotype is typically IgG₁. Where such cytotoxic activity is not desirable, the constant domain may be of another isotype, e.g., IgG₂. An alternative humanized anti-CD40 antibody can comprise sequences from more than one immunoglobulin class or isotype, and selecting particular constant domains to optimize desired effector functions is within the ordinary skill in the art. In specific embodiments, the present invention provides antibodies that are IgG1 antibodies and more particularly, are IgG1 antibodies in which there is a knock-out of effector functions.

The FRs and CDRs, or HVLs, of a humanized anti-CD40 antibody need not correspond precisely to the parental sequences. For example, one or more residues in the import CDR, or HVL, or the consensus or germline FR sequence may be altered (e.g., mutagenized) by substitution, insertion or deletion such that the resulting amino acid residue is no longer identical to the original residue in the corresponding position in either parental sequence but the antibody nevertheless retains the function of binding to CD40. Such alteration typically will not be extensive and will be conservative alterations. Usually, at least 75% of the humanized antibody residues will correspond to those of the parental consensus or germline FR and import CDR sequences, more often at least 90%, and most frequently greater than 95%, or greater than 98% or greater than 99%.

Immunoglobulin residues that affect the interface between heavy and light chain variable regions (“the V_{L}-V_{H} interface”) are those that affect the proximity or orientation of the two chains with respect to one another. Certain residues that may be involved in interchain interactions include V_{L} residues 34, 36, 38, 44, 46, 87, 89, 91, 96, and 98 and V_{H} residues 35, 37, 39, 45, 47, 91, 93, 95, 100, and 103 (utilizing the numbering system set forth in Kabat et al., Sequences of Proteins of Immunological Interest (National Institutes of Health, Bethesda, Md., 1987)). U.S. Pat. No. 6,407,213 also discusses that residues such as V_{L} residues 43 and 85, and V_{H} residues 43 and 60 also may be involved in this interaction. While these residues are indicated for human IgG only, they are applicable across species. Important antibody residues that are reasonably expected to be involved in interchain interactions are selected for substitution into the consensus sequence.

The terms “consensus sequence” and “consensus antibody” refer to an amino acid sequence which comprises the most frequently occurring amino acid residue at each location in all immunoglobulins of any particular class, isotype, or subunit structure, e.g., a human immunoglobulin variable domain. The consensus sequence may be based on immunoglobulins of a particular species or of many species. A “consensus” sequence, structure, or antibody is understood to encompass a consensus human sequence as described in certain embodiments, and to refer to an amino acid sequence which comprises the most frequently occurring amino acid residues at each location in all human immunoglobulins of any particular class, isotype, or subunit structure. Thus, the consensus sequence contains an amino acid sequence having at each position an amino acid that is present in one or more known immunoglobulins, but which may not exactly duplicate the entire amino acid sequence of any single immunoglobulin. The variable region consensus sequence is not obtained from any naturally produced antibody or immunoglobulin. Kabat et al., 1991, Sequences of Proteins of Immunological Interest, 5th Ed. Public Health Service, National Institutes of Health, Bethesda, Md., and variants thereof. The FRs of heavy and light chain consensus sequences, and variants thereof, provide useful sequences for the preparation of humanized anti-CD40 antibodies. See, for example, U.S. Pat. Nos. 6,037,454 and 6,054,297.

Human germline sequences are found naturally in human population. A combination of those germline genes generates antibody diversity. Germline antibody sequences for the light chain of the antibody come from conserved human germline kappa or lambda v-genes and j-genes. Similarly the heavy chain sequences come from germline v-, d- and j-genes (LeFranc, M-P, and LeFranc, G, “The Immunoglobulin Facts Book” Academic Press, 2001).

An “isolated” antibody is one that has been identified and separated and/or recovered from a component of its natural environment. Contaminant components of the antibody's natural environment are those materials that may interfere with diagnostic or therapeutic uses of the antibody, and can be enzymes, hormones, or other proteinaceous or nonproteinaceous solutes. In one aspect, the antibody will be purified to at least greater than 95% isolation by weight of antibody.

An isolated antibody includes an antibody in situ within recombinant cells in which it is produced, since at least one component of the antibody's natural environment will not be present. Ordinarily however, an isolated antibody will be prepared by at least one purification step in which the recombinant cellular material is removed.

The term “antibody performance” refers to factors that contribute to antibody recognition of antigen or the effectiveness of an antibody in vivo. Changes in the amino acid sequence of an antibody can affect antibody properties such as folding, and can influence physical factors such as initial rate of antibody binding to antigen (kₐ), dissociation constant of the antibody from antigen (k_{d}), affinity constant of the antibody for the antigen (Kd), conformation of the antibody, protein stability, and half-life of the antibody.

The antibodies also may be conjugated to prodrugs. A “prodrug” is a precursor or derivative form of a pharmaceutically active substance that is less cytotoxic to tumor cells compared to the parent drug and is capable of being enzymatically activated or converted into the more active form. See, for example, Wilman, 1986, “Prodrugs in Cancer Chemotherapy”, In Biochemical Society Transactions, 14, pp. 375-382, 615th Meeting Belfast and Stella et al., 1985, “Prodrugs: A Chemical Approach to Targeted Drug Delivery, In: “Directed Drug Delivery, Borchardt et al., (ed.), pp. 247-267, Humana Press.

For diagnostic as well as therapeutic monitoring purposes, the antibodies used in the method of the invention also may be conjugated to a label, either a label alone or a label and an additional second agent (prodrug and the like). A label, as distinguished from the other second agents refers to an agent that is a detectable compound or composition and it may be conjugated directly or indirectly to a humanized antibody of the present invention. The label may itself be detectable (e.g., radioisotope labels or fluorescent labels) or, in the case of an enzymatic label, may catalyze chemical alteration of a substrate compound or composition that is detectable. Labeled humanized anti-CD40 antibody can be prepared and used in various applications including in vitro and in vivo diagnostics.

The antibodies used in the method of the present invention may be formulated as part of a liposomal preparation in order to effect delivery thereof in vivo. A “liposome” is a small vesicle composed of various types of lipids, phospholipids, and/or surfactant. Liposomes are useful for delivery to a mammal of a compound or formulation, such as a humanized anti-CD40 antibody disclosed herein, optionally, coupled to or in combination with one or more pharmaceutically active agents and/or labels. The components of the liposome are commonly arranged in a bilayer formation, similar to the lipid arrangement of biological membranes.

The term “mammal” for purposes of treatment refers to any animal classified as a mammal, including humans, domesticated and farm animals, and zoo, sports, or pet animals, such as dogs, horses, cats, cows, and the like. Preferably, the mammal is human.

A “disorder”, as used herein, is any condition that would benefit from treatment with a humanized anti-CD40 antibody described herein. This includes chronic and acute disorders or diseases including those pathological conditions that predispose the mammal to the disorder in question. Non-limiting examples or disorders to be treated herein include cancer, hematological malignancies, benign and malignant tumors, leukemias and lymphoid malignancies and inflammatory, angiogenic, autoimmune and immunologic disorders.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia.

As used herein, the term “CD40-associated disorder” or “CD40-associated disease” refers to a condition in which modification or elimination of cells expressing CD40 is indicated. These include CD40-expressing cells demonstrating abnormal proliferation or CD40-expressing cells that are associated with cancerous or malignant growth. More particular examples of cancers that demonstrate abnormal expression of CD40 antigen include B lymphoblastoid cells, Burkitt's lymphoma, multiple myeloma, T cell lymphomas, Kaposi's sarcoma, osteosarcoma, epidermal and endothelial tumors, pancreatic, lung, breast, ovarian, colon, prostate, head and neck, skin (melanoma), bladder, and kidney cancers. Such disorders include, but are not limited to, leukemias, lymphomas, including B cell lymphoma and non-Hodgkin's lymphoma, multiple myeloma, Waldenstrom's macroglobulinemia; solid tumors, including sarcomas, such as osteosarcoma, Ewing's sarcoma, malignant melanoma, adenocarcinoma, including ovarian adenocarcinoma, Kaposi's sarcoma/Kaposi's tumor and squamous cell carcinoma.

A CD40-associated disorder also includes diseases and disorders of the immune system, such as autoimmune disorders and inflammatory disorders. Such conditions include, but are not limited to, lupus nephritis, rheumatoid arthritis (RA), systemic lupus erythematosus (SLE), scleroderma, Sjogren's syndrome, multiple sclerosis, psoriasis, inflammatory bowel disease (e.g., ulcerative colitis and Crohn's disease), pulmonary inflammation, asthma, and idiopathic thrombocytopenic purara (ITP).

The phrase “arrests the growth of” or “growth inhibitory” when used herein refers to inhibiting growth or proliferation of a cell, especially a neoplastic cell type expressing the CD40 antigen. Thus, growth inhibition, for example, significantly reduces the percentage of neoplastic cells in S phase.

The term “intravenous infusion” refers to introduction of an agent into the vein of an animal or human patient over a period of time greater than approximately 15 minutes, generally between approximately 30 to 90 minutes.

The term “intravenous bolus” or “intravenous push” refers to drug administration into a vein of an animal or human such that the body receives the drug in approximately 15 minutes or less, generally 5 minutes or less.

The term “subcutaneous administration” refers to introduction of an agent under the skin of an animal or human patient, preferable within a pocket between the skin and underlying tissue, by relatively slow, sustained delivery from a drug receptacle. Pinching or drawing the skin up and away from underlying tissue may create the pocket.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is less than approximately 15 minutes; in another aspect, less than 5 minutes, and in still another aspect, less than 60 seconds. In yet even another aspect, administration is within a pocket between the skin and underlying tissue, where the pocket may be created by pinching or drawing the skin up and away from underlying tissue.

The term “therapeutically effective amount” is used to refer to an amount of an active agent that relieves or ameliorates one or more of the symptoms of the disorder being treated. In doing so it is that amount that has a beneficial patient outcome, for example, a growth arrest effect or causes the deletion of the cell. In one aspect, the therapeutically effective amount has apoptotic activity, or is capable of inducing cell death. In another aspect, the therapeutically effective amount refers to a target serum concentration that has been shown to be effective in, for example, slowing disease progression. Efficacy can be measured in conventional ways, depending on the condition to be treated. For example, in neoplastic diseases or disorders characterized by cells expressing CD40, efficacy can be measured by assessing the time to disease progression, or determining the response rates.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the humanized CD40 antibody composition, the result should be considered an effective treatment of the underlying disorder regardless of whether all the symptoms of the disorder are alleviated or not.

The term “package insert” is used to refer to instructions customarily included in commercial packages of therapeutic products, that contain information about the indications, usage, administration, contraindications and/or warnings concerning the use of such therapeutic products.

### Antibodies

The humanized anti-CD40 antibodies and binding agents can be used in the treatment and/or prevention a variety of diseases or disorders characterized by the proliferation of cells expressing the CD40 surface antigen such as lupus nephritis. A humanized anti-CD40 antibody and a CD40 binding agent each includes at least a portion that specifically recognizes a CD40 epitope (i.e., an antigen-binding fragment).

Methods for making the anti-CD40 antibodies have been previously described in US20110243932, the entire contents of which are incorporated herein by reference.

As previously described in US20110243932, the initial characterization murine antibodies were selected based on CD40 binding characterization.

From these initial studies, murine antibodies were selected that had the following heavy chain variable regions shown in Table 1 and the light chain variable regions shown in Table 2:

TABLE 1
CD40 Murine Leads-VH Sequences

| 2H11 | EVQLQQSGAELVRPGASVKLSCTASGFNIKDYYVHWVKQRPEKG |
|---|---|
| | LEWIGRIDPEDGDSKYAPKFQGKATMTADTSSNTAYLHLSSLTS |
| | EDTAVYYCTTSYYVGTYGYWGQGTTLTVSS (SEQ ID NO: |
| | 1) |
| 10F2 | EVQLQQSGAELVRPGASVKLSCTASGFNIKDYYIHWVKQRPEKG |
| | LEIWIGRDPEDGDTKYDPKFQGKATMTADTSSNTAYLHLSSLTS |
| | EDTAVYYCTTSYYVGTYGYWGQGTTLTVSS (SEQ ID NO: |
| | 2) |
| 19B10 | EVQLQQSGAELVRPGASVQLSCTASGFNIKDYYVHWVKQRPEKG |
| | LEWIGRIDPEDGDTKFAPKFQGKATMTADTSSNTVYLHLSSLTS |
| | EDTAVYYCTTSYYVGTYGYWGQGTTLTVSS (SEQ ID NO: |
| | 3) |
| 20E2 | EVQLVESGGGLVKPGGSRKLSCAASGFTFSDYGMHWVRQAPEKG |
| | WLEVAYISSGNRIIYYADTVKGRFTISRDNAKNTLFLQMTSLRS |
| | EDTALYYCARQDGYRYAMDYWGQGTSVTVSS (SEQ ID NO: |
| | 4) |

TABLE 2
CD40 Murine Leads-VK Sequences

| | |
|---|---|
| 2H11 | QIVLTQSPAIMSASPGEKVTITCSASSSVSYMLWFQQKPGTSPK |
| | ILWYSTSNLASGVPARFGGSGSGTSYSLTISRMEAEDAATYYCQ |
| | QRTFYPYTFGGGTKLEIK (SEQ ID NO: 5) |
| 10F2 | QIVLTQSPTIMSASPGEKVIITCSATSSVSYILWFQQKPGTSPK |
| | LWIYSTSNLASGVPARFSGSGSGASYSLTISRMEAEDAATYYCQ |
| | QRTFYPYTFGGGTKLEIK (SEQ ID NO: 6) |
| 19B10 | QIVLTQSPAIMSASPGEKVTITCSASSSVSYMLWFQQKPGTSPK |
| | ILWYSTSNLASGVPARFSGSGSGTSYSLTISRMEAEDAATYYCQ |
| | QRTFYPYTFGGGTKLEIK (SEQ ID NO: 7) |
| 20E2 | DIVMTQSPSSLTVTAGEKVTMSCKSSQSLLNSGNQKNYLTWHQQ |
| | KPGQPPKLLIYWTSTRESGVPDRFTGSGSGTDFTLTISNLQAED |
| | LAVYYCQNDYTYPLTFGAGTKLELK (SEQ ID NO: 8) |

Human framework sequences were selected for each of the mouse leads based on the framework homology, CDR structure, conserved canonical residues, conserved interface packing residues and other parameters.

The murine heavy chain and light chain CDRs of the various murine antibodies selected antibodies are shown in Table 3 and Table 4, respectively:

TABLE 3
HEAVY CHAIN CDR sequences

| Construct | | | |
|---|---|---|---|
| name | H-CDR1 | H-CDR2 | H-CDR3 |
| 2H11 | GFNIKDYYVH | RIDPEDGDSKYAPKFQG | SYYVGTYGY |
| | SEQ ID NO: 9 | SEQ ID NO: 12 | SEQ ID NO: 16 |
| 10F2 | GFNIKDYYIH | RIDPEDGDTKYDPKFQG | SYYVGTYGY |
| | SEQ ID NO: 10 | SEQ ID NO: 13 | SEQ ID NO: 16 |
| 19B10 | GFNIKDYYVH | RIDPEDGDTKFAPKFQG | SYYVGTYGY |
| | SEQ ID NO: 9 | SEQ ID NO: 14 | SEQ ID NO: 16 |
| 20E2 | GFTFSDYGMH | YISSGNRIIYYADTVKG | QDGYRYAMDY |
| | SEQ ID NO: 11 | SEQ ID NO: 15 | SEQ ID NO: 17 |

The H-CDR1 listed above is using the sequence using the Chothia numbering system (AI-Lazikani et al., (1997) JMB 273,927-948). The Kabats numbering for the sequences is denoted by the bold italicized text and the IMGT numbering is shown by underlined text of the residues in the above table for CDR1 and CDR2. The sequences for the H-CDR3 for each of 2H11, 10F2 and 19B10 is

(SEQ ID NO: 77)
TTSYYVGTYGY

### and for 20E2 is

(SEQ ID NO: 78)
ARQDGYRYAMDY

TABLE 4
LIGHT CHAIN CDR sequences

| Construct | | | |
|---|---|---|---|
| name | L-CDR1 | L-CDR2 | L-CDR3 |
| 2H11 | SASSSVSYML | STSNLAS | QQRTFYPYT |
| | SEQ ID NO: 18 | SEQ ID NO: 22 | SEQ ID NO: 24 |
| 10F2 | SATSSVSYIL | STSNLAS | QQRTFYPYT |
| | SEQ ID NO: 19 | SEQ ID NO: 22 | SEQ ID NO: 24 |
| 19B10 | SASSSVSYML | STSNLAS | QQRTFYPYT |
| | SEQ ID NO: 20 | SEQ ID NO: 22 | SEQ ID NO: 24 |
| 20E2 | KSSQSLLNSGNQKNYLT | WTSTRES | QNDYTYPLT |
| | SEQ ID NO: 21 | SEQ ID NO: 23 | SEQ ID NO: 25 |

Again, the Chothia numbering system is used in Table 4 with the Kabats numbering for the sequences being denoted by the bold, italicized text and the IMGT numbering is shown by underlined text.

Fabs that showed better or equal binding as compared to the chimeric parent Fab were selected for conversion to IgG. Clones from the 20E2 series were converted to two different IgG formats: a) IgG4DM (double mutant) has two mutations in the Fc/hinge region, Ser228Pro which reduces half-molecule formation and Leu235Glu which further reduces FcγR binding. b) IgG1KO (knock-out of effector functions) has two mutations in the Fc region, Leu234Ala and Leu235Ala, which reduce effector function such as FcγR and complement binding. Both IgG formats are described in the literature. Example 1 describes the humanization of three candidates in further detail. The results of such humanization resulted in humanized antibody sequences, which have the heavy and light chain sequences shown below:

| Identity | Sequence | SEQ ID NO: |
|---|---|---|
| Antibody A | DIVMTQSPDSLAVSLGERATMSCKSSQSLLNSGNQKNYLTW | 26 |
| (Light Chain) | HQQKPGQPPKLLIYWTSTRESGVPDRFSGSGSGTDFTLTIS | |
| | SLQAEDVAVYYCQNDYTYPLTFGGGTKVEIKRTVAAPSVFI | |
| | FPPSDEQLKSGTASVVCLLNNFYPREAKVQWKVDNALQSGN | |
| | SQESVTEQDSKDSTYSLSSTLTLSKADYEKHKVYACEVTHQ | |
| | GLSSPVTKSFNRGEC | |
| Antibody A | EVQLVESGGGLVKPGGSLRLSCAASGFTFSDYGMHWVRQAP | 27 |
| (Heavy Chain, | GKGLEWVAYISSGNRIIYYADTVKGRFTISRDNAKNSLYLQ | |
| IgG1KO) | MNSLRAEDTALYYCARQDGYRYAMDYWAQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody A | EVQLVESGGGLVKPGGSLRLSCAASGFTFSDYGMHWVRQAP | 28 |
| (Heavy Chain, | GKGLEWVAYISSGNRIIYYADTVKGRFTISRDNAKNSLYLQ | |
| IgG1) | MNSLRAEDTALYYCARQDGYRYAMDYWAQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody A | EVQLVESGGGLVKPGGSLRLSCAASGFTFSDYGMHWVRQAP | 29 |
| (Heavy Chain, | GKGLEWVAYISSGNRIIYYADTVKGRFTISRDNAKNSLYLQ | |
| IgG4DM) | MNSLRAEDTALYYCARQDGYRYAMDYWAQGTLVTVSSASTK | |
| | GPSVFPLAPCSRSTSESTAALGCLVKDYFPEPVTVSWNSGA | |
| | LTSGVHTFPAVLQSSGLYSLSSVVTVPSSSLGTKTYTCNVD | |
| | HKPSNTKVDKRVESKYGPPCPPCPAPEFEGGPSVFLEPPKP | |
| | KDTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVK | |
| | GFYPSDIAVEWESNGQPENNYKTTPPVLDSDGSFFLYSRLT | |
| | VDKSRWQEGNVESCSVMHEALHNHYTQKSLSLSLGK | |
| Antibody A | EVQLVESGGGLVKPGGSLRLSCAASGFTFSDYGMHWVRQAP | 30 |
| (Heavy, IgG1KOb) | GKGLEWVAYISSGNRIIYYADTVKGRFTISRDNAKNSLYLQ | |
| | MNSLRAEDTALYYCARQDGYRYAMDYWAQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSRDELTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody B | DIVMTQSPDSLAVSLGEKVTINCKSSQSLLNSGNQKNYL | 31 |
| (Light Chain) | TWHQQKPGQPPKLLIYWTSTRESGVPDRFSGSGSGTDFT | |
| | LTISSLQAEDVAVYYCQNDYTYPLTFGGGTKVEIKRTVA | |
| | APSVFIFPPSDEQLKSGTASVVCLLNNFYPREAKVQWKV | |
| | DNALQSGNSQESVTEQDSKDSTYSLSSTLTLSKADYEKH | |
| | KVYACEVTHQGLSSPVTKSFNRGEC | |
| Antibody B | EVQLVESGGGLVKPGGSLRLSCAASGFTFSDYGMHWVRQAP | 32 |
| (Heavy Chain, | GKGLEWVAYISSGNRIIYYADTVKGRFTISRDNAKNSLYLQ | |
| IgG1KO) | MNSLRAEDTAVYYCARQDGYRYAMDYWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody B | EVQLVESGGGLVKPGGSLRLSCAASGFTFSDYGMHWVRQAP | 33 |
| (Heavy Chain, | GKGLEWVAYISSGNRIIYYADTVKGRFTISRDNAKNSLYLQ | |
| IgG1) | MNSLRAEDTAVYYCARQDGYRYAMDYWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody B | EVQLVESGGGLVKPGGSLRLSCAASGFTFSDYGMHWVRQAP | 34 |
| (Heavy | GKGLEWVAYISSGNRIIYYADTVKGRFTISRDNAKNSLYLQ | |
| Chain, IgG4 DM) | MNSLRAEDTAVYYCARQDGYRYAMDYWGQGTLVTVSSASTK | |
| | GPSVFPLAPCSRSTSESTAALGCLVKDYFPEPVTVSWNSGA | |
| | LTSGVHTFPAVLQSSGLYSLSSVVTVPSSSLGTKTYTCNVD | |
| | HKPSNTKVDKRVESKYGPPCPPCPAPEFEGGPSVFLEPPKP | |
| | KDTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVK | |
| | GFYPSDIAVEWESNGQPENNYKTTPPVLDSDGSFFLYSRLT | |
| | VDKSRWQEGNVESCSVMHEALHNHYTQKSLSLSLGK | |
| Antibody B | EVQLVESGGGLVKPGGSLRLSCAASGFTFSDYGMHWVRQAP | 35 |
| (Heavy Chain, | GKGLEWVAYISSGNRIIYYADTVKGRFTISRDNAKNSLYLQ | |
| IgG1KOb) | MNSLRAEDTAVYYCARQDGYRYAMDYWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSRDELTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody C | DIQMTQSPSSLSASVGDRVTITCSASSSVSYMLWFQ | 36 |
| (Light Chain) | QKPGKAPKLLIYSTSNLASGVPSRFSGSGSGTDFTL | |
| | TISSLQPEDFATYYCQQRTFYPYTFGGGTKVEIKRT | |
| | VAAPSVFIFPPSDEQLKSGTASVVCLLNNFYPREAK | |
| | VQWKVDNALQSGNSQESVTEQDSKDSTYSLSSTLTL | |
| | SKADYEKHKVYACEVTHQGLSSPVTKSFNRGEC | |
| Antibody C | QVQLVQSGAEVKKPGASVKVSCTASGFNIKDYYVHWVKQAP | 37 |
| (Heavy Chain, | GQGLEWMGRIDPEDGDSKYAPKFQGKATMTADTSTSTVYME | |
| IgG1KO) | LSSLRSEDTAVYYCTTSYYVGTYGYWGQGTLVTVSSASTKG | |
| | PSVFPLAPSSKSTSGGTAALGCLVKDYFPEPVTVSWNSGAL | |
| | TSGVHTFPAVLQSSGLYSLSSVVTVPSSSLGTQTYICNVNH | |
| | KPSNTKVDKRVEPKSCDKTHTCPPCPAPEAAGGPSVFLFPP | |
| | KPKDTLMISRTPEVTCVVVDVSHEDPEVKFNWYVDGVEVHN | |
| | AKTKPREEQYNSTYRVVSVLTVLHQDWLNGKEYKCKVSNKA | |
| | LPAPIEKTISKAKGQPREPQVYTLPPSREEMTKNQVSLTCL | |
| | VKGFYPSDIAVEWESNGQPENNYKTTPPVLDSDGSFFLYSK | |
| | LTVDKSRWQQGNVESCSVMHEALHNHYTQKSLSLSPGK | |
| Antibody C | QVQLVQSGAEVKKPGASVKVSCTASGFNIKDYYVHWVKQAP | |
| (Heavy Chain, | GQGLEWMGRIDPEDGDSKYAPKFQGKATMTADTSTSTVYME | 38 |
| IgG1) | LSSLRSEDTAVYYCTTSYYVGTYGYWGQGTLVTVSSASTKG | |
| | PSVFPLAPSSKSTSGGTAALGCLVKDYFPEPVTVSWNSGAL | |
| | TSGVHTFPAVLQSSGLYSLSSVVTVPSSSLGTQTYICNVNH | |
| | KPSNTKVDKRVEPKSCDKTHTCPPCPAPELLGGPSVFLFPP | |
| | KPKDTLMISRTPEVTCVVVDVSHEDPEVKFNWYVDGVEVHN | |
| | AKTKPREEQYNSTYRVVSVLTVLHQDWLNGKEYKCKVSNKA | |
| | LPAPIEKTISKAKGQPREPQVYTLPPSREEMTKNQVSLTCL | |
| | VKGFYPSDIAVEWESNGQPENNYKTTPPVLDSDGSFFLYSK | |
| | LTVDKSRWQQGNVESCSVMHEALHNHYTQKSLSLSPGK | |
| Antibody C | QVQLVQSGAEVKKPGASVKVSCTASGFNIKDYYVHWVKQAP | 39 |
| (Heavy | GQGLEWMGRIDPEDGDSKYAPKFQGKATMTADTSTSTVYME | |
| Chain, IgG4 DM) | LSSLRSEDTAVYYCTTSYYVGTYGYWGQGTLVTVSSASTKG | |
| | PSVFPLAPCSRSTSESTAALGCLVKDYFPEPVTVSWNSGAL | |
| | TSGVHTFPAVLQSSGLYSLSSVVTVPSSSLGTKTYTCNVDH | |
| | KPSNTKVDKRVESKYGPPCPPCPAPEFEGGPSVFLEPPKPK | |
| | DTLMISRTPEVTCVVVDVSQEDPEVQFNWYVDGVEVHNAKT | |
| | KPREEQFNSTYRVVSVLTVLHQDWLNGKEYKCKVSNKGLPS | |
| | SIEKTISKAKGQPREPQVYTLPPSQEEMTKNQVSLTCLVKG | |
| | FYPSDIAVEWESNGQPENNYKTTPPVLDSDGSFFLYSRLTV | |
| | DKSRWQEGNVESCSVMHEALHNHYTQKSLSLSLGK | |
| Antibody C | QVQLVQSGAEVKKPGASVKVSCTASGFNIKDYYVHWVKQAP | 40 |
| (Heavy Chain, | GQGLEWMGRIDPEDGDSKYAPKFQGKATMTADTSTSTVYME | |
| IgG1KOb) | LSSLRSEDTAVYYCTTSYYVGTYGYWGQGTLVTVSSASTKG | |
| | PSVFPLAPSSKSTSGGTAALGCLVKDYFPEPVTVSWNSGAL | |
| | TSGVHTFPAVLQSSGLYSLSSVVTVPSSSLGTQTYICNVNH | |
| | KPSNTKVDKRVEPKSCDKTHTCPPCPAPEAAGGPSVFLFPP | |
| | KPKDTLMISRTPEVTCVVVDVSHEDPEVKFNWYVDGVEVHN | |
| | AKTKPREEQYNSTYRVVSVLTVLHQDWLNGKEYKCKVSNKA | |
| | LPAPIEKTISKAKGQPREPQVYTLPPSRDELTKNQVSLTCL | |
| | VKGFYPSDIAVEWESNGQPENNYKTTPPVLDSDGSFFLYSK | |
| | LTVDKSRWQQGNVESCSVMHEALHNHYTQKSLSLSPGK | |

In some embodiments, the antigen-binding fragment can, for example, block proliferation or otherwise arrest the growth of a cell or cause its depletion, death, or otherwise its deletion, for example, through binding the CD40 surface antigen. For example, in T and B cell malignancies, anti-tumor effects (e.g., growth arrest with or without cell deletion or apoptosis) often result when malignant cells are exposed to stimuli that lead to activation of normal lymphocytes. This activation-induced growth arrest has been observed with signals through either antigen receptors or costimulatory receptors (see, e.g., Ashwell et al., 1987, Science 237:61; Bridges et al., 1987, J. Immunol. 139:4242; Page and Defranco, 1988, J. Immunol. 140:3717; and Beckwith et al., 1990, J. Natl. Cancer Inst. 82:501). CD40 stimulation, as a result of specific binding by either antibody or soluble ligand, inhibits B cell lymphoma growth (see, e.g., Funakoshi et al., 1994, Blood 83:2787-2794). Agents that inhibit malignant cell growth in this way and that are directed against the CD40 surface antigen are examples of appropriate agents.

CD40 specific agents include an antigen-binding fragment of a humanized anti-CD40 antibody that binds to CD40 (e.g., human CD40 or a variant thereof). The CD40 specific agents and antibodies can be optionally conjugated with or fused to a cytotoxic or chemotherapeutic agent. In aspects where the humanized antibody binds to the CD40 surface antigen and causes depletion of the CD40 expressing cell types, binding is generally characterized by homing to the CD40 surface antigen cell in vivo. Suitable binding agents bind the CD40 antigen with sufficient affinity and/or avidity such that the CD40 specific agent is useful as a therapeutic agent by specifically targeting a cell expressing the antigen.

In some aspects, the humanized antibody decreases the binding of CD40 ligand to CD40 by at least 45%, by at least 50%, by at least 60% or by at least 75% or at least 80%, or at least 90%, or at least 95%.

In some embodiments, the humanized anti-CD40 antibodies, including antigen-binding fragments thereof, such as heavy and light chain variable domains, comprise an amino acid sequence of the residues derived from the CDRs Antibody A (heavy chain sequence=SEQ ID NO:27; SEQ ID NO:28; SEQ ID NO:29 or SEQ ID NO:30; light chain sequence=SEQ ID NO:26), Antibody B (heavy chain sequence=SEQ ID NO:32; SEQ ID NO:33; SEQ ID NO:34; or SEQ ID NO:35; light chain sequence=SEQ ID NO:31) and Antibody C (heavy chain sequence=SEQ ID NO:37; SEQ ID NO:38; SEQ ID NO:39 or SEQ ID NO:40; light chain sequence=SEQ ID NO:36;) described herein above and amino acid residues derived from framework regions of a human immunoglobulin. The humanized anti-CD40 antibodies optionally include specific amino acid substitutions in the consensus or germline framework regions.

The specific substitution of amino acid residues in these framework positions can improve various aspects of antibody performance including binding affinity and/or stability, over that demonstrated in humanized antibodies formed by “direct swap” of CDRs or HVLs into the human germline framework regions, as shown in the examples below.

In some embodiments, the present invention describes other monoclonal antibodies with heavy chain (VH) sequences of SEQ ID NO:1 through SEQ ID NO:4 and light chain (VL) sequences of SEQ ID NO:5 to SEQ ID NO:8 (see Tables 1 and 2 above). The CDR sequence of these murine antibodies are shown in Tables 3 and 4 placing such CDRs into FRs of the human consensus heavy and light chain variable domains will yield useful humanized antibodies of the present invention.

In some specific embodiments, the humanized anti-CD40 antibodies disclosed herein comprise at least a heavy or light chain variable domain comprising the CDRs or HVLs of the murine monoclonal antibodies as shown in Tables 1 through 4 above and the FRs of the human germline heavy and light chain variable domains. In exemplary embodiments, the humanized antibodies created herein are: Antibody A, Antibody B and Antibody C and the various heavy and light chain sequences of the same are shown in SEQ ID NOs 26 through SEQ ID NO:40.

In specific embodiments, antibodies are contemplated that have a heavy chain sequence of any of SEQ ID NO: 27, SEQ ID NO:28, SEQ ID NO:29 or SEQ ID NO:30 in combination with a light chain sequence of SEQ ID NO:26. Alternative antibodies include those that have a heavy chain sequence of SEQ ID NO:32, SEQ ID NO:33, SEQ ID NO:34 or SEQ ID NO SEQ ID NO:35, in combination with a light chain sequence of SEQ ID NO:31. In still additional embodiments, there are provided humanized antibodies that have a heavy chain sequence of SEQ ID NO: 37, SEQ ID NO:38; SEQ ID NO:39 or SEQ ID NO: 40, in combination with a light chain sequence of SEQ ID NO:36.

The CDRs of these sequences are shown in Tables 3 and 4. In specific embodiments, it is contemplated that chimerical antibodies with switched CDR regions (i.e., for example switching one or two CDRs of Antibody A with the analogous CDR from Antibody C) between these exemplary immunoglobulins may yield useful antibodies.

In certain embodiments, the humanized anti-CD40 antibody is an antibody fragment. Various antibody fragments have been generally discussed above and there are techniques that have been developed for the production of antibody fragments. Fragments can be derived via proteolytic digestion of intact antibodies (see, e.g., Morimoto et al., 1992, Journal of Biochemical and Biophysical Methods 24:107-117; and Brennan et al., 1985, Science 229:81). Alternatively, the fragments can be produced directly in recombinant host cells. For example, Fab′-SH fragments can be directly recovered from E. coli and chemically coupled to form F(ab′)₂ fragments (see, e.g., Carter et al., 1992, Bio/Technology 10:163-167). By another approach, F(ab′)₂ fragments can be isolated directly from recombinant host cell culture. Other techniques for the production of antibody fragments will be apparent to the skilled practitioner.

Certain embodiments include an F(ab′)₂ fragment of a humanized anti-CD40 antibody comprising a have a heavy chain sequence of any of SEQ ID NO:27, SEQ ID NO:28, SEQ ID NO:29 or SEQ ID NO:30 in combination with a light chain sequence of SEQ ID NO:26. Alternative antibodies include those that have a heavy chain sequence of SEQ ID NO:32, SEQ ID NO:33, SEQ ID NO:34 or SEQ ID NO:35, in combination with a light chain sequence of SEQ ID NO:31. In still additional embodiments, there are provided humanized antibodies that have a heavy chain sequence of SEQ ID NO: 37, SEQ ID NO:38; SEQ ID NO:39 or SEQ ID NO: 40, in combination with a light chain sequence of SEQ ID NO:36. Such embodiments can include an intact antibody comprising such an F(ab′)₂.

In some embodiments, the antibody or antibody fragment includes a constant region that mediates effector function. The constant region can provide antibody-dependent cellular cytotoxicity (ADCC), antibody-dependent cellular phagocytosis (ADCP) and/or complement-dependent cytotoxicity (CDC) responses against a CD40-expressing target cell. The effector domain(s) can be, for example, an Fc region of an Ig molecule. Typically, the CD40 binding agent recruits and/or activates cytotoxic white blood cells (e.g., natural killer (NK) cells, phagocytotic cells (e.g., macrophages), and/or serum complement components).

The effector domain of an antibody can be from any suitable vertebrate animal species and isotypes. The isotypes from different animal species differ in the abilities to mediate effector functions. For example, the ability of human immunoglobulin to mediate CDC and ADCC/ADCP is generally in the order of IgM≈IgG₁≈IgG₃>IgG₂>IgG₄ and IgG₁≈IgG₃>IgG₂/IgM/IgG₄, respectively. Murine immunoglobulins mediate CDC and ADCC/ADCP generally in the order of murine IgM≈IgG₃>>IgG_{2b}>IgG₂ₐ>>IgG₁ and IgG_{2b}>IgG₂ₐ>IgG₁>>IgG₃, respectively. In another example, murine IgG₂a mediates ADCC while both murine IgG₂a and IgM mediate CDC.

### Antibody Modifications

The humanized anti-CD40 antibodies and agents can include modifications of the humanized anti-CD40 antibody or antigen-binding fragment thereof.

Conjugates of the humanized anti-CD40 antibody can be made by known methods, using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., 1987, Science 238:1098. Carbon-14-labeled 1-isothiocyanatobenzyl-3-methyldiethylene triaminepentaacetic acid (MX-DTPA) is an exemplary chelating agent for conjugation of radionucleotide to the antibody. Conjugates also can be formed with a cleavable linker.

The humanized anti-CD40 antibodies disclosed herein can also be formulated as immunoliposomes. Liposomes containing the antibody are prepared by methods known in the art, such as described in Epstein et al., 1985, Proc. Natl. Acad. Sci. USA 82:3688; Hwang et al., 1980, Proc. Natl. Acad. Sci. USA 77:4030; and U.S. Pat. Nos. 4,485,045 and 4,544,545. Liposomes having enhanced circulation time are disclosed, for example, in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody disclosed herein can be conjugated to the liposomes as described in Martin et al., 1982, J. Biol. Chem. 257:286-288 via a disulfide interchange reaction.

In other embodiments, covalent modifications of the humanized anti-CD40 antibody are also included. Covalent modifications include modification of cysteinyl residues, histidyl residues, lysinyl and amino-terminal residues, arginyl residues, tyrosyl residues, carboxyl side groups (aspartyl or glutamyl), glutaminyl and asparaginyl residues, or seryl, or threonyl residues. Another type of covalent modification involves chemically or enzymatically coupling glycosides to the antibody. Such modifications may be made by chemical synthesis or by enzymatic or chemical cleavage of the antibody, if applicable. Other types of covalent modifications of the antibody can be introduced into the molecule by reacting targeted amino acid residues of the antibody with an organic derivatizing agent that is capable of reacting with selected side chains or the amino- or carboxy-terminal residues.

Removal of any carbohydrate moieties present on the antibody can be accomplished chemically or enzymatically. Chemical deglycosylation is described by Hakimuddin et al., 1987, Arch. Biochem. Biophys. 259:52 and by Edge et al., 1981, Anal. Biochem., 118:131. Enzymatic cleavage of carbohydrate moieties on antibodies can be achieved by the use of a variety of endo- and exo-glycosidases as described by Thotakura et al., 1987, Meth. Enzymol 138:350.

Another type of useful covalent modification comprises linking the antibody to one of a variety of nonproteinaceous polymers, e.g., polyethylene glycol, polypropylene glycol, or polyoxyalkylenes, in the manner set forth in one or more of U.S. Pat. Nos. 4,640,835, 4,496,689, 4,301,144, 4,670,417, 4,791,192 and 4,179,337.

### Humanization and Amino Acid Sequence Variants

Amino acid sequence variants of the anti-CD40 antibody can be prepared by introducing appropriate nucleotide changes into the anti-CD40 antibody DNA, or by peptide synthesis. Such variants include, for example, deletions from, and/or insertions into and/or substitutions of, residues within the amino acid sequences of the anti-CD40 antibodies of the examples herein. Any combination of deletions, insertions, and substitutions is made to arrive at the final construct, provided that the final construct possesses the desired characteristics. The amino acid changes also may alter post-translational processes of the humanized or variant anti-CD40 antibody, such as changing the number or position of glycosylation sites.

A useful method for identification of certain residues or regions of the anti-CD40 antibody that are preferred locations for mutagenesis is called “alanine scanning mutagenesis,” as described by Cunningham and Wells (Science, 244:1081-1085 (1989)). Here, a residue or group of target residues are identified (e.g., charged residues such as arg, asp, his, lys, and glu) and replaced by a neutral or negatively charged amino acid (typically alanine) to affect the interaction of the amino acids with CD40 antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed anti-CD40 antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-CD40 antibody fused to an epitope tag. Other insertional variants of the anti-CD40 antibody molecule include a fusion to the N- or C-terminus of the anti-CD40 antibody of an enzyme or a polypeptide which increases the serum half-life of the antibody.

Another type of variant is an amino acid substitution variant. These variants have at least one amino acid residue in the anti-CD40 antibody molecule removed and a different residue inserted in its place. The sites of greatest interest for substitutional mutagenesis include the hypervariable regions, but FR alterations are also contemplated. Conservative substitutions are shown in Table 5 under the heading of “preferred substitutions”. If such substitutions result in a change in biological activity, then more substantial changes, denominated “exemplary substitutions”, or as further described below in reference to amino acid classes, may be introduced and the products screened.

TABLE 5

| | | Preferred |
|---|---|---|
| Original Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | arg; asn; gln; lys; | arg |
| Ile (I) | leu; val; met; ala; phe; norleucine | leu |
| Leu (L) | ile; norleucine; val; met; ala; phe | ile |
| Lys (K) | arg; gln; asn | arg |
| Met (M) | leu; phe; ile | leu |
| Phe (F) | tyr; leu; val; ile; ala; | tyr |
| Pro (P) | ala | ala |
| Ser (S) | thr | thr |
| Thr (T) | ser | ser |
| Trp (W) | tyr; phe | tyr |
| Tyr (Y) | phe; trp; thr; ser | phe |
| Val (V) | leu; ile; met; phe ala; norleucine; | leu |

In protein chemistry, it is generally accepted that the biological properties of the antibody can be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the humanized or variant anti-CD40 antibody also may be substituted, generally with serine, to improve the oxidative stability of the molecule, prevent aberrant crosslinking, or provide for established points of conjugation to a target compound. Conversely, cysteine bond(s) may be added to the antibody to improve its stability (particularly where the antibody is an antibody fragment such as an Fv fragment).

A type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity). In order to identify candidate hypervariable region sites for modification, alanine scanning mutagenesis can be performed to identify hypervariable region residues contributing significantly to antigen binding. Alternatively, or in addition, it may be beneficial to analyze a crystal structure of the antigen-antibody complex to identify contact points between the antibody and human CD40. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By “altering” is meant deleting one or more carbohydrate moieties found in the antibody, and/or adding one or more glycosylation sites that are not present in the antibody.

In some embodiments, it may be desirable to modify the antibodies of the invention to add glycosylations sites. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Thus, in order to glycosylate a given protein, e.g., an antibody, the amino acid sequence of the protein is engineered to contain one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-CD40 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-CD40 antibody.

### Therapeutic Uses

The humanized anti-CD40 antibody or agent is administered by any suitable means, including parenteral, subcutaneous, intraperitoneal, intrapulmonary, and intranasal, and, if desired for local immunosuppressive treatment, intralesional administration (including perfusing or otherwise contacting the graft with the antibody before transplantation). The humanized anti-CD40 antibody or agent can be administered, for example, as an infusion or as a bolus. Parenteral infusions include intramuscular, intravenous, intraarterial, intraperitoneal, or subcutaneous administration. In addition, the humanized anti-CD40 antibody is suitably administered by pulse infusion, particularly with declining doses of the antibody. In one aspect, the dosing is given by injections, most preferably intravenous or subcutaneous injections, depending in part on whether the administration is brief or chronic.

For the prevention or treatment of disease, the appropriate dosage of antibody will depend on a variety of factors such as the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to 20 mg/kg (e.g., 0.1-15 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen is that disclosed in WO 94/04188.

The term “suppression” is used herein in the same context as “amelioration” and “alleviation” to mean a lessening of one or more characteristics of the disease.

The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat the disorder associated with CD40 expression.

The antibody need not be, but is optionally, formulated with one or more agents currently used to prevent or treat the disorder in question. The effective amount of such other agents depends on the amount of humanized anti-CD40 antibody present in the formulation, the type of disorder or treatment, and other factors discussed above. These are generally used in the same dosages and with administration routes as used hereinbefore or about from 1 to 99% of the heretofore employed dosages.

### CD40-Associated Disorders

The anti-CD40 antibodies or agents are useful for treating or preventing a CD40-expressing cancer or an immunological disorder characterized by expression of CD40, e.g., by inappropriate activation of immune cells (e.g., lymphocytes or dendritic cells). Such expression of CD40 can be due to, for example, increased CD40 protein levels on the cells surface and/or altered antigenicity of the expressed CD40. Treatment or prevention of the immunological disorder, according to the methods described herein, is achieved by administering to a subject in need of such treatment or prevention an effective amount of the anti-CD40 antibody or agent, whereby the antibody (i) binds to activated immune cells that express CD40 and that are associated with the disease state and (ii) exerts a cytotoxic, cytostatic, or immunosuppressive effect on the activated immune cells.

Immunological diseases that are characterized by inappropriate activation of immune cells and that can be treated or prevented by the methods described herein can be classified, for example, by the type(s) of hypersensitivity reaction(s) that underlie the disorder. These reactions are typically classified into four types: anaphylactic reactions, cytotoxic (cytolytic) reactions, immune complex reactions, or cell-mediated immunity (CMI) reactions (also referred to as delayed-type hypersensitivity (DTH) reactions). (See, e.g., Fundamental Immunology (William E. Paul ed., Raven Press, N.Y., 3rd ed. 1993).)

Specific examples of such immunological diseases include the following: rheumatoid arthritis, autoimmune demyelinative diseases (e.g., multiple sclerosis, allergic encephalomyelitis), endocrine opthalmopathy, uveoretinitis, systemic lupus erythematosus, myasthenia gravis, Grave's disease, glomerulonephritis, autoimmune hepatological disorder, inflammatory bowel disease (e.g., Crohn's disease or ulcerative colitis), anaphylaxis, allergic reaction, Sjogren's syndrome, type I diabetes mellitus, primary biliary cirrhosis, Wegener's granulomatosis, fibromyalgia, polymyositis, dermatomyositis, inflammatory myositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nedosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, acute respiratory distress syndrome, pulmonary inflammation, osteoporosis, delayed type hypersensitivity and autoimmune gonadal failure.

Accordingly, the methods described herein encompass treatment of disorders of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes.

Rheumatoid arthritis (RA) is one of the most common inflammatory autoimmune diseases affecting approximately 1% of the population. While efficacious treatments (e.g. MTX and the anti-TNF agents) are available, there exists great unmet medical need, especially for those patients who do not adequately respond to anti-TNF therapies (about 30% of patients). In addition, up to 50% of patients discontinue TNF-antagonist treatment within 5 years, mainly due to adverse events but also because an increasingly recognized number of patients lose therapeutic benefit. It is thus important to establish effective therapies that target inflammation and joint destruction in RA but do not rely solely on the direct inhibition of TNF. A very attractive approach is to target costimulatory cell pathways. One of the key receptor-ligand pairs in costimulation is CD40/CD40L. This system allows interactions between immune cells, and between immune and non-immune cells, all of which are important in the pathogenesis of RA. Blockade of CD40 with an antagonistic antibody of the present invention may have one of more of the following effect in RA:
1) Inhibit B cell differentiation and antibody isotype switching;
2) Inhibit cytokine and chemokine production and up-regulation of adhesion molecules in T-cells and macrophages;
3) Inhibit the activation of dendritic cells and
4) Inhibit production of proinflammatory cytokines, chemokines, matrix metalloproteinases, prostaglandins, and down-regulate adhesion molecules in non-immune cells (e.g. epithelial, endothelial and mesenchymal cells).

Methods of achieving one of more of the above effects are expressly contemplated herein. In addition to RA, the compositions of the present invention will be particularly useful in methods of treatment of Multiple Sclerosis, Psoriasis (including Psoriatic Arthritis), Juvenile Rheumatoid Arthritis. Inflammatory Bowel Disease, Systemic Lupus Erythematosus, and Solid Organ Transplantation.

Rheumatoid Arthritis (RA) is a chronic, systemic autoimmune disease with a prevalence of approximately 1% in adults. The disease continues to cause significant morbidity and premature mortality (mortality is predominantly due to accelerated cardiovascular disease). It has now been identified that joint damage occurs very early in the course of the disease with up to 30% of patients showing radiographic evidence of bony erosions at the time of diagnosis, increasing to 60% after 1 year. Current guidelines recommend initiating therapy with traditional disease-modifying antirheumatic drugs (DMARDs) within 3 months after a definite diagnosis has been established. DMARDs have the potential to reduce or prevent joint damage and preserve joint function. Currently, rheumatologists select methotrexate (MTX) as the initial DMARD therapy for most patients.

The TNF-antagonists etanercept (Enbrel®), infliximab (Remicade®), adalimumab (Humira®), the CTLA4-antagonist abatacept (Orencia®), the anti-IL-6 receptor mAb tocilizumab and the anti-CD20 mAb rituximab (Rituxan®) are efficacious in the treatment of RA. Current guidelines generally recommend using biologic DMARDs for the treatment of active RA after an inadequate response to traditional DMARDs.

Recent studies in patients with early aggressive RA without previous MTX treatment showed that the combination of MTX with a TNF-antagonist was superior to each when used as monotherapy. The most striking result was the significant radiological benefit of the combination therapy. Thus, the combination of MTX and TNF-inhibitors should be used in patients at greatest risk for aggressive disease and aggressive phenotype (e.g. high activity score, functional impairment, seropositivity for rheumatoid factor (RF) or anti-cyclic citrullinated peptide antibody (CCP), elevated CRP, radiographic erosions). However, we anticipate that in clinical practice it will be rare that TNF-antagonists will be used as a first-line therapy. A survey of US rheumatologists conducted in April 2005 showed that the factors that most influence the decision to use a TNF-antagonist were: failure of MTX or multiple DMARDs, physician global assessment, functional impairment, and radiographic worsening or erosions. Currently, an estimated 20% of patients with RA receive TNF-inhibitor therapy in the US.

A substantial percentage of RA patients are not adequately helped with the current treatments including biologic therapies, either because of drug intolerance and toxicity or lack of response. Up to 50% of patients discontinue TNF-antagonist treatment within 5 years, mainly due to adverse events but also because an increasingly recognized number of patients lose their response.

In some embodiments, the immunological disorder is a T cell-mediated immunological disorder, such as a T cell disorder in which activated T cells associated with the disorder express CD40. Anti-CD40 antibodies or agents can be administered to deplete such CD40-expressing activated T cells. In a specific embodiment, administration of anti-CD40 antibodies or agents can deplete CD40-expressing activated T cells, while resting T cells are not substantially depleted by the anti-CD40 or agent. In this context, “not substantially depleted” means that less than about 60%, or less than about 70% or less than about 80% of resting T cells are not depleted.

The anti-CD40 antibodies and agents as described herein are also useful for treating or preventing a CD40-expressing cancer. Treatment or prevention of a CD40-expressing cancer, according to the methods described herein, is achieved by administering to a subject in need of such treatment or prevention an effective amount of the anti-CD40 antibody or agent, whereby the antibody or agent (i) binds to CD40-expressing cancer cells and (ii) exerts a cytotoxic or cytostatic effect to deplete or inhibit the proliferation of the CD40-expressing cancer cells.

CD40-expressing cancers that can be treated or prevented by the methods described herein include, for example, leukemia, such as acute leukemia, acute lymphocytic leukemia, acute myelocytic leukemia (e.g., myeloblastic, promyelocytic, myelomonocytic, monocytic, or erythroleukemia), chronic leukemia, chronic myelocytic (granulocytic) leukemia, or chronic lymphocytic leukemia; Polycythemia vera; Lymphoma (e.g., Hodgkin's disease or Non-Hodgkin's disease); multiple myeloma, Waldenstrom's macroglobulinemia; heavy chain disease; solid tumors such sarcomas and carcinomas (e.g., fibrosarcoma, myxosarcoma, liposarcoma, chondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, non small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, or esophageal carcinoma).

### Pharmaceutical Compositions and Administration Thereof

A composition comprising a CD40 binding agent (e.g., an anti-CD40 antibody) can be administered to a subject having or at risk of having an immunological disorder or a CD40-expressing cancer. The invention further provides for the use of a CD40 binding agent (e.g., an anti-CD40 antibody) in the manufacture of a medicament for prevention or treatment of a CD40 expressing cancer or immunological disorder. The term “subject” as used herein means any mammalian patient to which a CD40-binding agent can be administered, including, e.g., humans and non-human mammals, such as primates, rodents, and dogs. Subjects specifically intended for treatment using the methods described herein include humans. The antibodies or agents can be administered either alone or in combination with other compositions in the prevention or treatment of the immunological disorder or CD40-expressing cancer.

Preferred antibodies for use in such pharmaceutical compositions are those that comprise humanized antibody or antibody fragment having the heavy chain variable region amino acid sequence of any of SEQ ID NO: 1 to 4, SEQ ID NO:27, SEQ ID NO:28, SEQ ID NO: 29, SEQ ID NO:30, SEQ ID NO:32, SEQ ID NO:33, SEQ ID NO:34, SEQ ID NO:35, SEQ ID NO:37, SEQ ID NO:38, SEQ ID NO:39, or SEQ ID NO: 40.

In an embodiment, the present invention relates to a method of treating or preventing an autoimmune or inflammatory disease in a subject in need thereof, the method comprising administering to the subject a composition comprising an anti-CD40 (anti-cluster of differentiation 40) antibody,

wherein the anti-CD40 antibody comprises a heavy chain and a light chain, wherein the heavy chain sequence and light chain sequence are selected from the group consisting of: a) a heavy chain CDR1 sequence selected from the group consisting of SEQ ID NO: 9 through SEQ ID NO:11, a heavy chain CDR2 sequence selected from the group consisting of SEQ ID NO:12 through SEQ ID NO:15, and a heavy chain CDR3 sequence selected from the group consisting of SEQ ID NO:16 through SEQ ID NO:17; and b) the light chain CDR1 sequence has a sequence selected from the group consisting of SEQ ID NO:18 through SEQ ID NO:21, a light chain CDR2 sequence of SEQ ID NO:22 through SEQ ID NO:23, and a light chain CDR3 sequence selected from the group consisting of SEQ ID NO:24 through SEQ ID NO:25; or
wherein the anti-CD40 antibody comprises a variable heavy chain domain comprising any of SEQ ID NO:42, SEQ ID NO:44, SEQ ID NO:46, SEQ ID NO:48, SEQ ID NO:53, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73; and a variable light chain domain comprising any of SEQ ID NO:41, SEQ ID NO:43, SEQ ID NO:45, SEQ ID NO:47, SEQ ID NO:49, SEQ ID NO:50, SEQ ID NO:51, SEQ ID NO:52, SEQ ID NO:54, SEQ ID NO:55, SEQ ID NO:56, SEQ ID NO:74, SEQ ID NO:75, or SEQ ID NO:76; or
wherein the anti-CD40 antibody comprises a heavy chain sequence and a light chain sequence comprising the amino acid sequences of SEQ ID NO:27 and SEQ ID NO:26, respectively; SEQ ID NO:28 and SEQ ID NO:26, respectively; SEQ ID NO:29 and SEQ ID NO:26, respectively; SEQ ID NO:30 and SEQ ID NO:26, respectively; SEQ ID NO:32 and SEQ ID NO:31, respectively; SEQ ID NO:33 and SEQ ID NO:31, respectively; SEQ ID NO:34 and SEQ ID NO:31, respectively; SEQ ID NO:35 and SEQ ID NO:31, respectively; SEQ ID NO:37 and SEQ ID NO:36, respectively; SEQ ID NO:38 and SEQ ID NO:36, respectively; SEQ ID NO:39 and SEQ ID NO:36, respectively; or SEQ ID NO:40 and SEQ ID NO: 36, respectively;
wherein the composition comprises 80 mg, 120 mg, 180 mg or 240 mg of the anti-CD40 antibody;
wherein a single-dose administration of the composition comprising 80 mg of the anti-CD40 antibody results in Cₘₐₓ (ng mL⁻¹) of about 888 to about 1550, AUC_{0-tz} (μg·h mL⁻¹) of about 126 to about 365, or AUC_{0-inf} (μg·h mL⁻¹) of about 330 to about 464; or
wherein a single-dose administration of the composition comprising 120 mg of the anti-CD40 antibody results in Cₘₐₓ(ng mL⁻¹) of about 5160 to about 7210, AUC_{0-tz} (μg·h mL⁻¹) of about 1110 to about 2010, or AUC_{0-inf} (μg·h mL⁻¹) of about 1120 to about 2020; or
wherein a single-dose administration of the composition comprising 180 mg of the anti-CD40 antibody results in Cₘₐₓ (ng mL⁻¹) of about 8650 to about 16300, AUC_{0-tz} (μg·h mL⁻¹) of about 2900 to about 6380, or AUC_{0-inf} (μg·h mL⁻¹) of about 2020 to about 2910; or
wherein a single-dose administration of the composition comprising 240 mg of the anti-CD40 antibody results in Cₘₐₓ (ng mL⁻¹) of about 15700 to about 21300, AUC_{0-tz} (μg·h mL⁻¹) of about 5680 to about 7750, or AUC_{0-inf} (μg·h mL⁻¹) of about 5610 to about 7780; or
wherein a multiple-dose administration (q1w or once every week) of the composition comprising 240 mg of the anti-CD40 antibody results in Cmax,1 (μg mL−1) of about 23 after the first dose or AUC_{T}, 1 (μg·h mL−1) of about 2600 after the first dose; or
wherein a multiple-dose administration (q1w or once every week) of the composition comprising 240 mg of the anti-CD40 antibody results in Cmax,4 (μg mL−1) of about 74 after the frouth dose, or Cmin, 4(μg mL−1) of about 49 after the fourth dose, or AUC_{T}, 4 (μg·h mL−1) of about 10900 after the fourth dose.

The method according to the above embodiment, wherein the autoimmune or inflammatory disease is selected from the group consisting of lupus nephritis, rheumatoid arthritis, multiple sclerosis, proliferative lupus glomerulonephritis, inflammatory bowel disease (IBD), psoriasis, idiopathic thrombocytopenic purpura (ITP), Crohn's Disease and systemic lupus erythematosus (SLE), Hashimoto's thyroiditis, primary myxoedema, thyrotoxicosis/Graves disease, pernicious anaemia, autoimmune atrophic gastritis, autoimmune carditis, Addison's disease, premature menopause, type 1-diabetes mellitus, Good pasture's syndrome, myasthenia gravis, autoimmune haemolytic anaemia, idiopathic leucopenia, primary biliary cirrhosis, active chronic hepatitis (HBs Ag negative), cryptogenic cirrhosis, Sjogren's syndrome, dermatomyositis, scleroderma, mixed tissues connective disease, discoid lupus erythematosus, and systemic vasculitis.

The method according to the above embodiment, wherein the antibody comprises a heavy chain CDR1 sequence of SEQ ID NO: 10, a heavy chain CDR2 sequence of SEQ ID NO:13 and a heavy chain CDR3 sequence of SEQ ID NO:16; and wherein said antibody comprises a light chain CDR1 sequence of SEQ ID NO:19, a light chain CDR2 sequence of SEQ ID NO:22 and a light chain CDR3 sequence of SEQ ID NO:24.

The method according to the above embodiment, wherein said antibody comprises a heavy chain CDR1 sequence of SEQ ID NO: 9, a heavy chain CDR2 sequence of SEQ ID NO:14 and a heavy chain CDR3 sequence of SEQ ID NO:16; and wherein said antibody comprises a light chain CDR1 sequence of SEQ ID NO:20, a light chain CDR2 sequence of SEQ ID NO:22 and a light chain CDR3 sequence of SEQ ID NO:24.

The method according to the above embodiment, wherein said antibody comprises a heavy chain CDR1 sequence of SEQ ID NO: 11, a heavy chain CDR2 sequence of SEQ ID NO:15 and a heavy chain CDR3 sequence of SEQ ID NO:17; and wherein said antibody comprises a light chain CDR1 sequence of SEQ ID NO:21, a light chain CDR2 sequence of SEQ ID NO:23 and a light chain CDR3 sequence of SEQ ID NO:25.

The method according to the above embodiment, wherein the antibody comprises a heavy chain variable domain and a light chain variable region comprising the amino acid sequences of SEQ ID NO:27 and SEQ ID NO:26, respectively; SEQ ID NO:28 and SEQ ID NO:26, respectively; SEQ ID NO:29 and SEQ ID NO:26, respectively; SEQ ID NO:30 and SEQ ID NO:26, respectively; SEQ ID NO:32 and SEQ ID NO:31, respectively; SEQ ID NO:33 and SEQ ID NO:31, respectively; SEQ ID NO:34 and SEQ ID NO:31, respectively; SEQ ID NO:35 and SEQ ID NO:31, respectively; SEQ ID NO:37 and SEQ ID NO:36, respectively; SEQ ID NO:38 and SEQ ID NO:36, respectively; SEQ ID NO:39 and SEQ ID NO:36, respectively; SEQ ID NO:40 and SEQ ID NO: 36, respectively.

The method according to the above embodiment, wherein the antibody comprises: a heavy chain variable domain comprising SEQ ID NO:44 and a light chain variable domain comprising SEQ ID NO:43; or a heavy chain variable domain comprising SEQ ID NO:53 and a light chain variable domain comprising SEQ ID NO:52; or a heavy chain variable domain comprising SEQ ID NO:58 and a light chain variable domain comprising SEQ ID NO:56.

The method according to the above embodiment, wherein the antibody comprises: a heavy chain sequence comprising SEQ ID NO:30 and a light chain sequence comprising SEQ ID NO:26; or a heavy chain sequence comprising SEQ ID NO:35 and a light chain sequence comprising SEQ ID NO:31; or a heavy chain sequence comprising SEQ ID NO:40 and a light chain sequence comprising SEQ ID NO:36.

The method according to the above embodiment, wherein the autoimmune or inflammatory disease is selected from the group consisting of lupus nephritis, graft v. host disease, autoimmune or inflammatory disease, and CD40-related disorder.

In other embodiments, the compositions of the invention may be indicated for reducing signs and symptoms, inducing a major clinical response and reducing the progression of structural damage in patients with moderately to severely active RA who have had an inadequate response to anti-TNF agents. The current Gold standard: non-anti-TNF biologic therapy. Preferably, in such subjects the compositions of the invention possess non-inferior efficacy compared to non-anti-TNF biological (e.g. Orencia, Rituxan) by historical comparison in patients who have had an inadequate response to an anti-TNF agent: ACR20 at 6 months >50% for compound plus DMARD (GS: Orencia+DMARD 50% vs. placebo+DMARD 20%). In still other embodiments, the compositions of the invention inhibit progression of structural damage over a period of one year assessed by accepted X-ray scoring methods for joint erosion and joint space narrowing, similar to Rituxan (after 52 weeks mean modified Sharp score Rituxan+MTX 1.0 vs. Placebo+MTX 2.31).

Various delivery systems are known and can be used to administer the CD40 binding agent. Methods of introduction include but are not limited to intradermal, intramuscular, intraperitoneal, intravenous, subcutaneous, intranasal, epidural, and oral routes. The CD40 binding agent can be administered, for example by infusion, bolus or injection, and can be administered together with other biologically active agents such as chemotherapeutic agents. Administration can be systemic or local. In preferred embodiments, the administration is by subcutaneous injection. Formulations for such injections may be prepared in for example prefilled syringes that may be administered once every other week.

The safety characteristics of the antibodies of the invention will be determined and preferably include one or more features such as: no clinically significant adverse interactions with other medications commonly used to treat Rheumatoid Arthritis (e.g. DMARDs, Steroids, NSAIDs,); No greater rate of discontinuations due to safety or tolerability issues compared to Enbrel; Rate of serious infections no greater than anti-TNF agents or other commonly used biologic agents; Frequency and/or severity of injection site reactions or infusion reaction similar to Enbrel; No or minimal development of drug resistance (less than 5%) upon repeat cycles of therapy; No or minimal neutralizing antibodies; No evidence of enhanced platelet aggregation/activation that could lead to thromboembolic events in vivo or platelet/endothelial dysfunction that could lead to bleeding.

In specific embodiments, the CD40 binding agent composition is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the anti-CD40 antibody or agent does not absorb are used.

In other embodiments, the anti-CD40 antibody or agent is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

A CD40 binding agent (e.g., an anti-CD40 antibody) can be administered as pharmaceutical compositions comprising a therapeutically effective amount of the binding agent and one or more pharmaceutically compatible ingredients.

In typical embodiments, the pharmaceutical composition is formulated in accordance with routine procedures as a pharmaceutical composition adapted for intravenous or subcutaneous administration to human beings. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Further, the pharmaceutical composition can be provided as a pharmaceutical kit comprising (a) a container containing a CD40 binding agent (e.g., an anti-CD40 antibody) in lyophilized form and (b) a second container containing a pharmaceutically acceptable diluent (e.g., sterile water) for injection. The pharmaceutically acceptable diluent can be used for reconstitution or dilution of the lyophilized anti-CD40 antibody or agent. Optionally associated with such container(s) can be a notice in the form prescribed by a governmental agency regulating the manufacture, use or sale of pharmaceuticals or biological products, which notice reflects approval by the agency of manufacture, use or sale for human administration.

In one embodiment, the pharmaceutical composition comprises an aqueous composition having a concentration of anti-CD40 antibody from about 10 mg/ml to about 200 mg/ml; or from about 100 mg/ml to about 200 mg/ml; or from about 120 mg/ml to about 180 mg/ml; or about 120 mg/ml, 130 mg/ml, 140 mg/ml, 150 mg/ml, 160 mg/ml, 170 mg/ml, 180 mg/ml, 190 mg/ml or 200 mg/ml.

In addition to the anti-CD40 antibody, the pharmaceutical composition may further comprise a buffer, a stabilizing agent, and a, optionally, a pH adjusting agent. Nonlimiting examples of buffering agents include one or more salts such as sodium chloride, arginine hydrochloride, sodium thiocyanate, ammonium thiocyanate, ammonium sulfate, ammonium chloride, calcium chloride, zinc chloride and sodium acetate; or the salts of suitable acids such as acetic acid and amino acids. The buffering agent is added in an amount sufficient to provide a viscosity suitable for administering the formulation to the patient, for example, by injection. The pharmaceutical composition may comprise from about 100 mM up to about 200 mM of salt or buffer, or from about 120 mM up to about 180 mM salt or buffer. In one embodiment, the pharmaceutical composition comprises a buffer comprising sodium acetate at a concentration of from about 20 mM to about 30 mM and sodium chloride at a concentration of from about 120 mM to about 140 mM. In another embodiment, the pharmaceutical composition comprises a buffer comprising sodium acetate at a concentration of about 25 mM and sodium chloride at a concentration of about 130 mM.

A nonlimiting example of a suitable stabilizing agent is polysorbate 20 (Tween 20). The stabilizing agent is present in an amount sufficient to maintain the chemical and physical stability of the pharmaceutical composition. The pharmaceutical composition may comprise from about 0.001% to about 0.1% (w/v) of stabilizing agent; or from about 0.0015% to about 0.015% (w/v) of stabilizing agent; or about 0.01% (w/v) of stabilizing agent.

In one embodiment, the pharmaceutical composition comprises the anti-CD40 antibody in an amount from about 120 mg/ml to about 180 mg/ml; a buffer comprising sodium acetate at a concentration of from about 20 mM to about 30 mM and sodium chloride at a concentration of from about 120 mM to about 140 mM; and a surfactant which is polysorbate 20 at a concentration for from about 0.0015 to about 0.015% (w/v). In another embodiment, the anti-CD40 antibody formulation comprises the anti-CD40 antibody in an amount of about 120 mg/ml, 130 mg/ml, 140 mg/ml, 150 mg/ml, 160 mg/ml, 170 mg/ml, 180 mg/ml, 190 mg/ml or 200 mg/ml; a buffer comprising sodium acetate at a concentration of about 25 mM and sodium chloride at a concentration of about 130 mM; and a surfactant which is polysorbate 20 at a concentration of about 0.01% (w/v).

In another embodiment, each of the pharmaceutical compositions described above may comprise from about 70 mg to about 250 mg of the anti-CD40 antibody; or from about 80 to 240 mg of the anti-CD40 antibody. In another embodiment, pharmaceutical compositions described above comprise 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, or 250 mg of the anti-CD40 antibody.

Each of the pharmaceutical compositions described above has a pH of from about 4.0 to about 12.0; or from about 5 to about 6.0; or about 5.5. The pH may be adjusted by addition of a sufficient amount of a suitable pH adjusting agent such as an acid (e.g., hydrochloric acid) or base (e.g., sodium hydroxide).

In another embodiment, the invention relates to a method of using any one of the ant-CD40 antibody pharmaceutical compositions described herein for treating or prevention lupus nephritis.

The amount of the CD40 binding agent (e.g., anti-CD40 antibody) that is effective in the treatment or prevention of an immunological disorder or CD40-expressing cancer can be determined by standard clinical techniques. In addition, in vitro assays may optionally be employed to help identify optimal dosage ranges. The precise dose to be employed in the formulation will also depend on the route of administration, and the stage of immunological disorder or CD40-expressing cancer, and should be decided according to the judgment of the practitioner and each patient's circumstances. Effective doses may be extrapolated from dose-response curves derived from in vitro or animal model test systems.

For example, toxicity and therapeutic efficacy of the anti-CD40 antibody or agent can be determined in cell cultures or experimental animals by standard pharmaceutical procedures for determining the ED₅₀ (the dose therapeutically effective in 50% of the population). A CD40-binding agent (e.g., an anti-CD40 antibody) that exhibits a large therapeutic index is preferred. Where a CD40-binding agent exhibits toxic side effects, a delivery system that targets the CD40-binding agent to the site of affected tissue can be used to minimize potential damage non-CD40-expressing cells and, thereby, reduce side effects.

The data obtained from the cell culture assays and animal studies can be used in formulating a range of dosage for use in humans. The dosage of the CD40 binding agent typically lies within a range of circulating concentrations that include the ED₅₀ with little or no toxicity. The dosage may vary within this range depending upon the dosage form employed and the route of administration utilized. For any CD40 binding agent used in the method, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography, ELISA and the like.

Generally, the dosage of an anti-CD40 antibody or CD40 binding agent administered to a patient with an immunological disorder or CD40-expressing cancer is typically about 0.1 mg/kg to about 100 mg/kg of the subject's body weight. The dosage administered to a subject is about 0.1 mg/kg to about 50 mg/kg, about 1 mg/kg to about 30 mg/kg, about 1 mg/kg to about 20 mg/kg, about 1 mg/kg to about 15 mg/kg, or about 1 mg/kg to about 10 mg/kg of the subject's body weight.

Exemplary doses include, but are not limited to, from 1 ng/kg to 100 mg/kg. In some embodiments, a dose is about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg or about 16 mg/kg. The dose can be administered, for example, daily, once per week (weekly), twice per week, thrice per week, four times per week, five times per week, six times per week, biweekly or monthly, every two months, or every three months. In specific embodiments, the dose is about 0.5 mg/kg/week, about 1 mg/kg/week, about 2 mg/kg/week, about 3 mg/kg/week, about 4 mg/kg/week, about 5 mg/kg/week, about 6 mg/kg/week, about 7 mg/kg/week, about 8 mg/kg/week, about 9 mg/kg/week, about 10 mg/kg/week, about 11 mg/kg/week, about 12 mg/kg/week, about 13 mg/kg/week, about 14 mg/kg/week, about 15 mg/kg/week or about 16 mg/kg/week. In some embodiments, the dose ranges from about 1 mg/kg/week to about 15 mg/kg/week.

In another embodiment, the dose is from about 70 mg to about 250 mg per week; or from about 80 to 240 mg per week. In another embodiment, the dose is about 80 mg per week, 120 mg per week, 130 mg per week, 140 mg per week, 160 mg per week, 170 mg per week, 180 mg per week, 200 mg per week, 210 mg per week, 220 mg per week, mg per week, 240 mg per week, or 250 mg per week.

In some embodiments, the pharmaceutical compositions comprising the CD40 binding agent can further comprise a therapeutic agent, either conjugated or unconjugated to the binding agent. The anti-CD40 antibody or CD40 binding agent can be co-administered in combination with one or more therapeutic agents for the treatment or prevention of immunological disorders or CD40-expressing cancers. For example, combination therapy can include a cytostatic, cytotoxic, or immunosuppressive agent. Combination therapy can also include, e.g., administration of an agent that targets a receptor or receptor complex other than CD40 on the surface of activated lymphocytes, dendritic cells or CD40-expressing cancer cells. An example of such an agent includes a second, non-CD40 antibody that binds to a molecule at the surface of an activated lymphocyte, dendritic cell or CD40-expressing cancer cell. Another example includes a ligand that targets such a receptor or receptor complex. Typically, such an antibody or ligand binds to a cell surface receptor on activated lymphocytes, dendritic cell or CD40-expressing cancer cell and enhances the cytotoxic or cytostatic effect of the anti-CD40 antibody by delivering a cytostatic or cytotoxic signal to the activated lymphocyte, dendritic cell or CD40-expressing cancer cell.

Such combination therapy administration can have an additive or synergistic effect on disease parameters (e.g., severity of a symptom, the number of symptoms, or frequency of relapse).

With respect to therapeutic regimens for combinatorial administration, in a specific embodiment, an anti-CD40 antibody or CD40 binding agent is administered concurrently with a therapeutic agent. In another specific embodiment, the therapeutic agent is administered prior or subsequent to administration of the anti-CD40 antibody or CD40 binding agent, by at least an hour and up to several months, for example at least an hour, five hours, 12 hours, a day, a week, a month, or three months, prior or subsequent to administration of the anti-CD40 antibody or CD40 binding agent.

Useful classes of cytotoxic or immunosuppressive agents include, for example, antitubulin agents, auristatins (e.g., MMAE, or MMAF), DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like.

Individual cytotoxic or immunosuppressive agents include, for example, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

In some typical embodiments, the therapeutic agent is a cytotoxic agent. Suitable cytotoxic agents include, for example, dolastatins (e.g., auristatin E, AFP, MMAF, MMAE, AEB or AEVB), DNA minor groove binders (e.g., enediynes and lexitropsins), duocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, or mitoxantrone.

In some embodiments, the cytotoxic agent is a conventional chemotherapeutic such as, for example, doxorubicin, paclitaxel, melphalan, vinca alkaloids, methotrexate, mitomycin C or etoposide. In addition, potent agents such as CC-1065 analogues, calicheamicin, maytansine, analogues of dolastatin 10, rhizoxin, and palytoxin can be linked to the anti-CD40 antibodies or agents thereof.

In specific embodiments, the cytotoxic or cytostatic agent is auristatin E (also known in the art as dolastatin-10) or a derivative thereof. Typically, the auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatin derivatives include AFP, MMAF, and MMAE. The synthesis and structure of auristatin E and its derivatives are described in, for example, U.S. Patent Application Publication Nos. 2004-0157782 A1 and 2005-0238649; International Patent Application No. PCT/US03/24209, International Patent Application No. PCT/US02/13435, and U.S. Pat. Nos. 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414; the disclosures of which are incorporated by reference herein.

In specific embodiments, the cytotoxic agent is a DNA minor groove binding agent. (See, e.g., U.S. Pat. No. 6,130,237.) For example, in some embodiments, the minor groove binding agent is a CBI compound. In other embodiments, the minor groove binding agent is an enediyne (e.g., calicheamicin).

Examples of anti-tubulin agents include, but are not limited to, taxanes (e.g., Taxol® (paclitaxel), Taxotere® (docetaxel)), T67 (Tularik), vinca alkyloids (e.g., vincristine, vinblastine, vindesine, and vinorelbine), and dolastatins (e.g., auristatin E, AFP, MMAF, MMAE, AEB, AEVB). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin.

In some embodiments, the cytotoxic agent is a maytansinoid, another group of anti-tubulin agents. For example, in specific embodiments, the maytansinoid is maytansine or DM-1 (ImmunoGen, Inc.; see also Chari et al., 1992, Cancer Res. 52:127-131).

In some embodiments, the therapeutic agent is not a radioisotope.

In some embodiments, the cytotoxic or immunosuppressive agent is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscamet, or trifluridine.

In other embodiments, the cytotoxic or immunosuppressive agent is tacrolimus, cyclosporine or rapamycin. In further embodiments, the cytotoxic agent is aldesleukin, alemtuzumab, alitretinoin, allopurinol, altretamine, amifostine, anastrozole, arsenic trioxide, bexarotene, bexarotene, calusterone, capecitabine, celecoxib, cladribine, Darbepoetin alfa, Denileukin diftitox, dexrazoxane, dromostanolone propionate, epirubicin, Epoetin alfa, estramustine, exemestane, Filgrastim, floxuridine, fludarabine, fulvestrant, gemcitabine, gemtuzumab ozogamicin, goserelin, idarubicin, ifosfamide, imatinib mesylate, Interferon alfa-2a, irinotecan, letrozole, leucovorin, levamisole, meclorethamine or nitrogen mustard, megestrol, mesna, methotrexate, methoxsalen, mitomycin C, mitotane, nandrolone phenpropionate, oprelvekin, oxaliplatin, pamidronate, pegademase, pegaspargase, pegfilgrastim, pentostatin, pipobroman, plicamycin, porfimer sodium, procarbazine, quinacrine, rasburicase, revlimid, Sargramostim, streptozocin, tamoxifen, temozolomide, teniposide, testolactone, thioguanine, toremifene, Tositumomab, Trastuzumab, tretinoin, uracil mustard, valrubicin, vinblastine, vincristine, vinorelbine and zoledronate.

In additional embodiments, the drug is a humanized anti-HER2 monoclonal antibody; RITUXAN (rituximab; Genentech, Inc., South San Francisco, Calif.); a chimeric anti-CD20 monoclonal antibody); OVAREX (AltaRex Corporation, MA); PANOREX (Glaxo Wellcome, NC; a murine IgG2a antibody); Cetuximab Erbitux (Imclone Systems Inc., NY; an anti-EGFR IgG chimeric antibody); Vitaxin (MedImmune, Inc., MD); Campath I/H (Leukosite, MA; a humanized IgG1 antibody); Smart M195 (Protein Design Labs, Inc., CA; a humanized anti-CD33 IgG antibody); LymphoCide (Immunomedics, Inc., NJ; a humanized anti-CD22 IgG antibody); Smart ID10 (Protein Design Labs, Inc., CA; a humanized anti-HLA-DR antibody); Oncolym (Techniclone, Inc., CA; a radiolabeled murine anti-HLA-Dr10 antibody); Allomune (BioTransplant, CA; a humanized anti-CD2 mAb); Avastin (Genentech, Inc., CA; an anti-VEGF humanized antibody); Epratuzamab (Immunomedics, Inc., NJ and Amgen, CA; an anti-CD22 antibody); and CEAcide (Immunomedics, NJ; a humanized anti-CEA antibody).

Other suitable antibodies include, but are not limited to, antibodies against the following antigens: CA125, CA15-3, CA19-9, L6, Lewis Y, Lewis X, alpha fetoprotein, CA 242, placental alkaline phosphatase, prostate specific antigen, prostatic acid phosphatase, epidermal growth factor, MAGE-1, MAGE-2, MAGE-3, MAGE-4, anti transferrin receptor, p97, MUC1-KLH, CEA, gp100, MART1, Prostate Specific Antigen, IL-2 receptor, CD20, CD52, CD33, CD22, human chorionic gonadotropin, CD38, mucin, P21, MPG, and Neu oncogene product.

In some embodiments, the additional therapeutic agent is an immunosuppressive agent. The immunosuppressive agent can be, for example, gancyclovir, etanercept, tacrolimus, cyclosporine, rapamycin, mycophenolate (MMF), cyclophosphamide (CyP), azathioprine, hydroxychloroquine, mizoribine, mycophenolate mofetil or methotrexate. Alternatively, the immunosuppressive agent can be, for example, a glucocorticoid (e.g., cortisol or aldosterone) or a glucocorticoid analogue (e.g., prednisone or dexamethasone). In another embodiment the immunosuppresive agent can be an angiotensin-converting enzyme (ACE) inhibitor (e.g., captopril, quinapril or enalapril) or an angiotensin II receptor blocker (ARB) (e.g., losartan or candesartan)

Suitable cyclooxygenase inhibitors include meclofenamic acid, mefenamic acid, carprofen, diclofenac, diflunisal, fenbufen, fenoprofen, ibuprofen, indomethacin, ketoprofen, nabumetone, naproxen, sulindac, tenoxicam, tolmetin, and acetylsalicylic acid.

Suitable lipoxygenase inhibitors include redox inhibitors (e.g., catechol butane derivatives, nordihydroguaiaretic acid (NDGA), masoprocol, phenidone, lanopalen, indazolinones, naphazatrom, benzofuranol, alkylhydroxylamine), and non-redox inhibitors (e.g., hydroxythiazoles, methoxyalkylthiazoles, benzopyrans and derivatives thereof, methoxytetrahydropyran, boswellic acids and acetylated derivatives of boswellic acids, and quinolinemethoxyphenylacetic acids substituted with cycloalkyl radicals), and precursors of redox inhibitors.

Other suitable lipoxygenase inhibitors include antioxidants (e.g., phenols, propyl gallate, flavonoids and/or naturally occurring substrates containing flavonoids, hydroxylated derivatives of the flavones, flavonol, dihydroquercetin, luteolin, galangin, orobol, derivatives of chalcone, 4,2′,4′-trihydroxychalcone, ortho-aminophenols, N-hydroxyureas, benzofuranols, ebselen and species that increase the activity of the reducing selenoenzymes), iron chelating agents (e.g., hydroxamic acids and derivatives thereof, N-hydroxyureas, 2-benzyl-1-naphthol, catechols, hydroxylamines, carnosol trolox C, catechol, naphthol, sulfasalazine, zyleuton, 5-hydroxyanthranilic acid and 4-(omega-arylalkyl)phenylalkanoic acids), imidazole-containing compounds (e.g., ketoconazole and itraconazole), phenothiazines, and benzopyran derivatives.

Yet other suitable lipoxygenase inhibitors include inhibitors of eicosanoids (e.g., octadecatetraenoic, eicosatetraenoic, docosapentaenoic, eicosahexaenoic and docosahexaenoic acids and esters thereof, PGE1 (prostaglandin E1), PGA2 (prostaglandin A2), viprostol, 15-monohydroxyeicosatetraenoic, 15-monohydroxy-eicosatrienoic and 15-monohydroxyeicosapentaenoic acids, and leukotrienes B5, C5 and D5), compounds interfering with calcium flows, phenothiazines, diphenylbutylamines, verapamil, fuscoside, curcumin, chlorogenic acid, caffeic acid, 5,8,11,14-eicosatetrayenoic acid (ETYA), hydroxyphenylretinamide, lonapalen, esculin, diethylcarbamazine, phenantroline, baicalein, proxicromil, thioethers, diallyl sulfide and di-(1-propenyl) sulfide.

Leukotriene receptor antagonists include calcitriol, ontazolast, Bayer Bay-x-1005, Ciba-Geigy CGS-25019C, ebselen, Leo Denmark ETH-615, Lilly LY-293111, Ono ONO-4057, Terumo TMK-688, Boehringer Ingleheim BI-RM-270, Lilly LY 213024, Lilly LY 264086, Lilly LY 292728, Ono ONO LB457, Pfizer 105696, Perdue Frederick PF 10042, Rhone-Poulenc Rorer RP 66153, SmithKline Beecham SB-201146, SmithKline Beecham SB-201993, SmithKline Beecham SB-209247, Searle SC-53228, Sumitamo SM 15178, American Home Products WAY 121006, Bayer Bay-o-8276, Warner-Lambert CI-987, Warner-Lambert CI-987BPC-15LY 223982, Lilly LY 233569, Lilly LY-255283, MacroNex MNX-160, Merck and Co. MK-591, Merck and Co. MK-886, Ono ONO-LB-448, Purdue Frederick PF-5901, Rhone-Poulenc Rorer RG 14893, Rhone-Poulenc Rorer RP 66364, Rhone-Poulenc Rorer RP 69698, Shionoogi S-2474, Searle SC-41930, Searle SC-50505, Searle SC-51146, Searle SC-52798, SmithKline Beecham SK and F-104493, Leo Denmark SR-2566, Tanabe T-757 and Teijin TEI-1338.

In another embodiment, the additional therapeutic agent selected from the group consisting of mycophenolate (MMF), cyclophosphamide (CyP), a glucocorticoid (GC), and corticosteroids, or any combination thereof.

In one embodiment, the additional therapeutic agent is mycophenolate (MMF).

In another embodiment, the additional therapeutic agent is cyclophosphamide (CyP).

In another embodiment, the additional therapeutic agent is a glucocorticoid (GC).

In another embodiment, the additional therapeutic agent is a corticosteroid.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the humanized anti-CD40 antibody. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

In an embodiment relating to any of the aspects or embodiments, the administration results in an improvement in total SLEDAI or non-renal SLEDAI scores in the subject as compared to placebo. In some embodiments, the improvement is ≥5% at Weeks 26 or 52. In some embodiments, the improvement is ≥10% at Weeks 26 or 52.

In another embodiment, the invention relates to a method of determining the treatment efficacy of an anti-CD40 antibody in treating or preventing an autoimmune or inflammatory disease in a subject, the method comprising administering to the subject a composition comprising the anti-CD40 antibody, measuring the levels of an activated B-cell subset in the subject, wherein a decrease in the levels of the activated B-cell subset (when comparing the levels before and after the treatment) is indicative of efficacy. In a related embodiment, the activated B-cell subset is selected from the group consisting of CD19⁺IgD⁻CD27⁻CD95⁺, CD19⁺IgD+CD27−CD95⁺, CD19⁺IgD⁻CD27⁺CD95⁺ and CD19⁺IgD⁺CD27⁺CD95⁺. In a related embodiment, the anti-CD40 antibody is any of those disclosed herein. In a related embodiment, the autoimmune or inflammatory disease is any of the ones disclosed herein.

In another embodiment, the invention relates to a method of decreasing the levels of an activated B-cell subset in a subject suffering from an autoimmune or inflammatory disease, the method comprising administering to the subject a composition comprising an anti-CD40 antibody, wherein the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺. In a related embodiment, the anti-CD40 antibody is any of the ones disclosed herein. In a related embodiment, the autoimmune or inflammatory disease is any of the ones disclosed herein.

In another embodiment, the invention relates to a method of treating or preventing an autoimmune or inflammatory disease in a subject in need thereof, the method comprising administering to the subject a composition comprising 80 mg, 120 mg, 180 mg or 240 mg of an anti-CD40 (anti-cluster of differentiation 40) antibody, wherein the subject exhibits (or has been determined to exhibit) the presence of an activated B-cell subset. In a related embodiment, the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺. In a related embodiment, the anti-CD40 antibody is any of those disclosed herein. In a related embodiment, the autoimmune or inflammatory disease is any of the ones disclosed herein.

In another embodiment, the invention relates to a method of treating or preventing an autoimmune or inflammatory disease in a subject in need thereof, the method comprising (a) determining that the subject exhibits the presence of an activated B-cell subset (e.g., by testing a biological sample obtained from the subject), (b) administering to the subject a composition comprising 80 mg, 120 mg, 180 mg or 240 mg of an anti-CD40 (anti-cluster of differentiation 40) antibody. In a related embodiment, the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺. In a related embodiment, the anti-CD40 antibody is any of those disclosed herein. In a related embodiment, the autoimmune or inflammatory disease is any of the ones disclosed herein.

In another embodiment, the invention relates to a method of treating or preventing an autoimmune or inflammatory disease in a subject in need thereof, the method comprising administering to the subject a composition comprising 80 mg, 120 mg, 180 mg or 240 mg of an anti-CD40 (anti-cluster of differentiation 40) antibody, wherein the subject has been determined to exhibit the presence of an activated B-cell subset. In a related embodiment, the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺. In a related embodiment, the anti-CD40 antibody is any of those disclosed herein. In a related embodiment, the autoimmune or inflammatory disease is any of the ones disclosed herein.

The invention is further described in the following examples, which are not intended to limit the scope of the invention.

### EXAMPLES

### Example 1: Production of Humanized Anti-CD40 Antibody

The humanized anti-CD4 antibodies of the invention cab be prepared according to the procedures described in US20110243932. Antibody A, Antibody B and Antibody C were humanized antibodies derived from mouse antibody 20E2 (Antibody A and Antibody B) or 2H11 (Antibody C) cloned into a human IgG₁-KO (KO=knock-out)/kappa backbone. IgG₁-KO has two mutations in the Fc region, Leu234Ala and Leu235Ala to reduce FcγR and complement binding.

The results of such humanization resulted in various humanized heavy and light chain variable sequences shown below:

SEQ ID NO: 41 (variable light chain sequence):
DIVMTQSPDSLAVSLGERVTMSCKSSQSLLNSGNQKNYLTWHQQKPGQPPKLLIYWTSTRESGVPDRFSGSGSGTDF
TLTISSLQAEDVAVYYCQNDYTYPLTFGGGTKVEIK
SEQ ID NO: 42 (variable heavy chain sequence):
EVQLVKSGGGLVKPGGSLRLSCAASGFTFSDYGMHWVRQAPGKGLEWVAYISSGNRIIYYADTVKGRFTISRDNAKN
SLYLQMNSLRAEDTALYYCARQDGYRYAMDYWGQGTLVTVSS
SEQ ID NO: 43 (variable light chain sequence)
DIVMTQSPDSLAVSLGERATMSCKSSQSLLNSGNQKNYLTWHQQKPGQPPKLLIYWTSTRESGVPDRFSGSGSGTDF
TLTISSLQAEDVAVYYCQNDYTYPLTFGGGTKVEIK
SEQ ID NO: 44 (variable heavy chain sequence)
EVQLVESGGGLVKPGGSLRLSCAASGFTFSDYGMHWVRQAPGKGLEWVAYISSGNRIIYYADTVKGRFTISRDNAKN
SLYLQMNSLRAEDTALYYCARQDGYRYAMDYWAQGTLVTVSS
SEQ ID NO: 45 (variable light chain sequence)
DIVMTQSPDSLAVSLGEKVTMNCKSSQSLLNSGNQKNYLTWHQQKPGQPPKLLIYWTSTRESGVPDRFSGSGSGTDF
TLTISSLQAEDVAVYYCQNDYTYPLTFGAGTKVEIK
SEQ ID NO: 46 (variable heavy chain sequence)
EVQLVESGGGLVKPGGSRRLSCAASGFTFSDYGMHWVRQAPGKGLEWVAYISSGNRIIYYADTVKGRFTISRDNAKN
SLYLQMNSLRAEDTALYYCARQDGYRYAMDYWGQGTLVTVSS.
SEQ ID NO: 47 (variable light chain sequence)
DIVMTQSPDSLAVSLGERVTMNCKSSQSLLNSGNQKNYLTWHQQKPGQPPKLLIYWTSTRESGVPDRFSGSGSGTDF
TLTISSLQAEDVAVYYCQNDYTYPLTFGGGTKVEIK
SEQ ID NO: 48 (variable heavy chain sequence)
EVQLVESGGGLVKPGGSLRLSCAASGFTFSDYGMHWVRQAPGKGLEWVAYISSGNRIIYYADTVKGRFTISRDNAKN
SLYLQMNSLRAEDTALYYCARQDGYRYAMDYWGQGTLVTVSS
SEQ ID NO: 49 (variable light chain sequence)
DIVMTQSPDSLAVSLGERVTMNCKSSQSLLNSGNQKNYLTWHQQKPGQPPKLLIYWTSTRESGVPDRFSGSGSGTDF
TLTISSLQAEDVAVYYCQNDYTYPLTFGAGTKVEIK
SEQ ID NO: 50 (variable light chain sequence)
EVQLVESGGGLVKPGGSRRLSCAASGFTFSDYGMHWVRQAPGKGLEWVAYISSGNRIIYYADTVKGRFTISRDNAKN
SLYLQMNSLRAEDTAVYYCARQDGYRYAMDYWGQGTLVTVSS
SEQ ID NO: 51 (variable light chain sequence)
DIVMTQSPDSLAVSLGEKVTMNCKSSQSLLNSGNQKNYLTWHQQKPGQPPKLLIYWTSTRESGVPDRFSGSGSGTDF
TLTISSLQAEDLAVYYCQNDYTYPLTFGAGTKVEIK.
SEQ ID NO: 52 (variable light chain sequence)
DIVMTQSPDSLAVSLGEKVTINCKSSQSLLNSGNQKNYLTWHQQKPGQPPKLLIYWTSTRESGVPDRFSGSGSGTDF
TLTISSLQAEDVAVYYCQNDYTYPLTFGGGTKVEIK
SEQ ID NO: 53 (variable heavy chain sequence)
EVQLVESGGGLVKPGGSLRLSCAASGFTFSDYGMHWVRQAPGKGLEWVAYISSGNRIIYYADTVKGRFTISRDNAKN
SLYLQMNSLRAEDTAVYYCARQDGYRYAMDYWGQGTLVTVSS
SEQ ID NO: 54 (variable light chain sequence)
QIQMTQSPSSLSASVGDRVTITCSASSSVSYMLWFQQKPGKAPKLWIYSTSNLASGVPARFSGSGSGTDFTLTISSL
QPEDFATYYCQQRTFYPYTFGGGTKVEIK
SEQ ID NO: 55 (variable light chain sequence)
DIQMTQSPSSLSASVGDRVTITCSASSSVSYMLWFQQKPGKAPKLLIYSTSNLASGVPARFSGSGSGTDFTLTISSL
QPEDFATYYCQQRTFYPYTFGGGTKVEIK
SEQ ID NO: 56 (variable light chain sequence)
DIQMTQSPSSLSASVGDRVTITCSASSSVSYMLWFQQKPGKAPKLLIYSTSNLASGVPSRFSGSGSGTDFTLTISSL
QPEDFATYYCQQRTFYPYTFGGGTKVEIK
SEQ ID NO: 57 (variable heavy chain sequence)
QVQLVQSGAEVKKPGASVKVSCTASGFNITDYYVHWVKQRPGQGLEWMGRIDPEDGDSKYAPKFQGKATMTADTSTS
TVYMELSSLRSEDTAVYYCTTSYYVGTYGYWGQGTLVTVSS.
SEQ ID NO: 58 (variable heavy chain sequence)
QVQLVQSGAEVKKPGASVKVSCTASGFNIKDYYVHWVKQAPGQGLEWMGRIDPEDGDSKYAPKFQGKATMTADTSTS
TVYMELSSLRSEDTAVYYCTTSYYVGTYGYWGQGTLVTVSS
SEQ ID NO: 59 (variable heavy chain sequence)
QVQLVQSGAEVKKPGASVKVSCTASGFNITDYYVHWVKQRPGQGLEWMGRIDPEDGDSKYAPKFQGKVTMTADTSTS
TVYMELSSLRSEDTAVYYCTTSYYVGTYGYWGQGTLVTVSS.
SEQ ID NO: 60 (variable heavy found sequence)
QVQLVQSGAEVKKPGASVKVSCTASGFNIKDYYVHWVKQAPGQGLEWIGRIDPEDGDSKYAPKFQGKATMTADTSTS
TVYMELSSLRSEDTAVYYCTTSYYVGTYGYWGQGTLVTVSS
SEQ ID NO: 61 (variable heavy sequence)
QVQLVQSGAEVKKPGASVKVSCTASGFNITDYYVHWVKQAPGQGLEWMGRIDPEDGDSKYAPKFQGKATMTADTSTS
TVYMELSSLRSEDTAVYYCTTSYYVGTYGYWGQGTLVTVSS
SEQ ID NO: 62 (variable heavy sequence):
QVQLVQSGAEVKKPGASVKVSCTASGFNITDYYVHWVKQRPGQGLEWMGRIDPEDGDTKFAPKFQGKATMTADTSTS
TVYMELSSLRSEDTAVYYCTTSYYVGTYGYWGQGTLVTVSS
SEQ ID NO: 63 (variable heavy sequence)
QVQLVQSGAEVKKPGASVKVSCTASGFNITDYYVHWVKQRPGQGLEWMGRIDPEDGDTKFAPKFQGKVTMTADTSTS
TVYMELSSLRSEDTAVYYCTTSYYVGTYGYWGQGTLVTVSS
SEQ ID NO: 64 (variable heavy sequence)
QVQLVQSGAEVKKPGASVKVSCTASGFNIKDYYVHWVKQAPGQGLEWIGRIDPEDGDTKFAPKFQGKATMTADTSTS
TVYMELSSLRSEDTAVYYCTTSYYVGTYGYWGQGTLVTVSS
SEQ ID NO: 65 (variable heavy sequence)
QVQLVQSGAEVKKPGASVKVSCTASGFNIKDYYVHWVKQAPGQGLEWMGRIDPEDGDTKFAPKFQGKATMTADTSTS
TVYMELSSLRSEDTAVYYCTTSYYVGTYGYWGQGTLVTVSS
SEQ ID NO: 66 (variable heavy sequence)
QVQLVQSGAEVKKPGASVKVSCTASGFNITDYYVHWVKQAPGQGLEWMGRIDPEDGDTKFAPKFQGKATMTADTSTS
TVYMELSSLRSEDTAVYYCTTSYYVGTYGYWGQGTLVTVSS
SEQ ID NO: 67 (variable heavy sequence)
EVQLVQSGAEVKKPGATVKISCKVSGFNIKDYYIHWVKQRPGKGLEWMGRIDPEDGDTKYDPKFQGRVTMTADTSTD
TAYMELSSLRSEDTAVYYCTTSYYVGTYGYWGQGTTVTVSS
SEQ ID NO: 68 (variable heavy sequence)
EVQLVQSGAEVKKPGATVKISCTVSGFNIKDYYIHWVKQRPGKGLEWMGRIDPEDGDTKYDPKFQGRVTMTADTSTD
TAYMELSSLRSEDTAVYYCTTSYYVGTYGYWGQGTTVTVSS
SEQ ID NO: 69 (variable heavy sequence)
EVQLVQSGAEVKKPGATVKISCTVSGFNIKDYYIHWVKQRPGKGLEWMGRIDPEDGDTKYDPKFQGKVTMTADTSTD
TAYMELSSLRSEDTAVYYCTTSYYVGTYGYWGQGTTVTVSS
SEQ ID NO: 70 (variable heavy sequence)
EVQLVQSGAEVKKPGATVKISCTVSGFNIKDYYIHWVKQAPGKGLEWMGRIDPEDGDTKYDPKFQGKATMTADTSTD
TAYMELSSLRSEDTAVYYCTTSYYVGTYGYWGQGTTVTVSS
SEQ ID NO: 71 (variable heavy sequence)
EVQLVQSGAEVKKPGATVKISCTVSGFNIKDYYIHWVKQRPGKGLEWMGRIDPEDGDTKYDPKFQGKATMTADTSTD
TAYMELSSLRSEDTAVYYCTTSYYVGTYGYWGQGTTVTVSS
SEQ ID NO: 72 (variable heavy sequence)
EVQLVQSGAEVKKPGATVKISCTVSGFNIKDYYIHWVKQAPGKGLEWIGRIDPEDGDTKYDPKFQGKATMTADTSTD
TAYMELSSLRSEDTAVYYCTTSYYVGTYGYWGQGTTVTVSS
SEQ ID NO: 73 (variable heavy sequence)
EVQLVQSGAEVKKPGATVKISCKVSGFNIKDYYIHWVQQAPGKGLEWMGRIDPEDGDTKYDPKFQGRVTMTADTSTD
TAYMELSSLRSEDTAVYYCTTSYYVGTYGYWGQGTTVTVSS
SEQ ID NO: 74 (variable light sequence) 1 from antibody 10F2Hum:
DIQMTQSPSSLSASVGDRVTITCSATSSVSYILWFQQKPGKAPKLLIYSTSNLASGVPSRFSGSGSGTDFTLTISSL
QPEDFATYYCQQRTFYPYTFGGGTKVEIK
SEQ ID NO: 75 (variable light sequence) 2 from antibody 10F2Hum:
DIQMTQSPSSLSASVGDRVTITCSATSSVSYILWFQQKPGKAPKLLIYSTSNLASGVPARFSGSGSGTDFTLTISSL
QPEDFATYYCQQRTFYPYTFGGGTKVEIK
SEQ ID NO: 76 (variable light sequence)
QIQMTQSPSSLSASVGDRVTITCSATSSVSYILWFQQKPGKAPKLWIYSTSNLASGVPARFSGSGSGTDFTLTISSL
QPEDFATYYCQQRTFYPYTFGGGTKVEIK

Exemplary humanized antibodies of the present invention are those that have the heavy and light chain sequences set forth in the following table. The bold underlined sequences in the following table are the variable domains whereas the normal, non-underlined sequences are the constant domains:

| Identity | Sequence | SEQ ID NO: |
|---|---|---|
| Antibody A | DIVMTQSPDSLAVSLGERATMSCKSSQSLLNSGNQKNYLTW | 26 |
| (Light Chain) | HQQKPGQPPKLLIYWTSTRESGVPDRFSGSGSGTDFTLTIS | |
| | SLQAEDVAVYYCQNDYTYPLTFGGGTKVEIKRTVAAPSVFI | |
| | FPPSDEQLKSGTASVVCLLNNFYPREAKVQWKVDNALQSGN | |
| | SQESVTEQDSKDSTYSLSSTLTLSKADYEKHKVYACEVTHQ | |
| | GLSSPVTKSFNRGEC | |
| Antibody A | EVQLVESGGGLVKPGGSLRLSCAASGFTFSDYGMHWVRQAP | 27 |
| (Heavy Chain, | GKGLEWVAYISSGNRIIYYADTVKGRFTISRDNAKNSLYLQ | |
| IgG1KO) | MNSLRAEDTALYYCARQDGYRYAMDYWAQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody A | EVQLVESGGGLVKPGGSLRLSCAASGFTFSDYGMHWVRQAP | 28 |
| (Heavy Chain, | GKGLEWVAYISSGNRIIYYADTVKGRFTISRDNAKNSLYLQ | |
| IgG1) | MNSLRAEDTALYYCARQDGYRYAMDYWAQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody A | EVQLVESGGGLVKPGGSLRLSCAASGFTFSDYGMHWVRQAP | 29 |
| (Heavy Chain, | GKGLEWVAYISSGNRIIYYADTVKGRFTISRDNAKNSLYLQ | |
| IgG4DM) | MNSLRAEDTALYYCARQDGYRYAMDYWAQGTLVTVSSASTK | |
| | GPSVFPLAPCSRSTSESTAALGCLVKDYFPEPVTVSWNSGA | |
| | LTSGVHTFPAVLQSSGLYSLSSVVTVPSSSLGTKTYTCNVD | |
| | HKPSNTKVDKRVESKYGPPCPPCPAPEFEGGPSVFLEPPKP | |
| | KDTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVK | |
| | GFYPSDIAVEWESNGQPENNYKTTPPVLDSDGSFFLYSRLT | |
| | VDKSRWQEGNVESCSVMHEALHNHYTQKSLSLSLGK | |
| Antibody A | EVQLVESGGGLVKPGGSLRLSCAASGFTFSDYGMHWVRQAP | 30 |
| (Heavy, IgG1KOb) | GKGLEWVAYISSGNRIIYYADTVKGRFTISRDNAKNSLYLQ | |
| | MNSLRAEDTALYYCARQDGYRYAMDYWAQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSRDELTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody B | DIVMTQSPDSLAVSLGEKVTINCKSSQSLLNSGNQKNYL | 31 |
| (Light Chain) | TWHQQKPGQPPKLLIYWTSTRESGVPDRFSGSGSGTDFT | |
| | LTISSLQAEDVAVYYCQNDYTYPLTFGGGTKVEIKRTVA | |
| | APSVFIFPPSDEQLKSGTASVVCLLNNFYPREAKVQWKV | |
| | DNALQSGNSQESVTEQDSKDSTYSLSSTLTLSKADYEKH | |
| | KVYACEVTHQGLSSPVTKSFNRGEC | |
| Antibody B | EVQLVESGGGLVKPGGSLRLSCAASGFTFSDYGMHWVRQAP | 32 |
| (Heavy Chain, | GKGLEWVAYISSGNRIIYYADTVKGRFTISRDNAKNSLYLQ | |
| IgG1KO) | MNSLRAEDTAVYYCARQDGYRYAMDYWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody B | EVQLVESGGGLVKPGGSLRLSCAASGFTFSDYGMHWVRQAP | 33 |
| (Heavy Chain, | GKGLEWVAYISSGNRIIYYADTVKGRFTISRDNAKNSLYLQ | |
| IgG1) | MNSLRAEDTAVYYCARQDGYRYAMDYWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody B | EVQLVESGGGLVKPGGSLRLSCAASGFTFSDYGMHWVRQAP | 34 |
| (Heavy | GKGLEWVAYISSGNRIIYYADTVKGRFTISRDNAKNSLYLQ | |
| Chain, IgG4 DM) | MNSLRAEDTAVYYCARQDGYRYAMDYWGQGTLVTVSSASTK | |
| | GPSVFPLAPCSRSTSESTAALGCLVKDYFPEPVTVSWNSGA | |
| | LTSGVHTFPAVLQSSGLYSLSSVVTVPSSSLGTKTYTCNVD | |
| | HKPSNTKVDKRVESKYGPPCPPCPAPEFEGGPSVFLEPPKP | |
| | KDTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVK | |
| | GFYPSDIAVEWESNGQPENNYKTTPPVLDSDGSFFLYSRLT | |
| | VDKSRWQEGNVESCSVMHEALHNHYTQKSLSLSLGK | |
| Antibody B | EVQLVESGGGLVKPGGSLRLSCAASGFTFSDYGMHWVRQAP | 35 |
| (Heavy Chain, | GKGLEWVAYISSGNRIIYYADTVKGRFTISRDNAKNSLYLQ | |
| IgG1KOb) | MNSLRAEDTAVYYCARQDGYRYAMDYWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKRVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSRDELTKNQVSLTC | |
| | LVKGFYPSDIAVEWESNGQPENNYKTTPPVLDSDGSFFLYS | |
| | KLTVDKSRWQQGNVESCSVMHEALHNHYTQKSLSLSPGK | |
| Antibody C | DIQMTQSPSSLSASVGDRVTITCSASSSVSYMLWFQ | 36 |
| (Light Chain) | QKPGKAPKLLIYSTSNLASGVPSRFSGSGSGTDFTL | |
| | TISSLQPEDFATYYCQQRTFYPYTFGGGTKVEIKRT | |
| | VAAPSVFIFPPSDEQLKSGTASVVCLLNNFYPREAK | |
| | VQWKVDNALQSGNSQESVIEQDSKDSTYSLSSTLTL | |
| | SKADYEKHKVYACEVTHQGLSSPVTKSFNRGEC | |
| Antibody C | QVQLVQSGAEVKKPGASVKVSCTASGFNIKDYYVHWVKQAP | 37 |
| (Heavy Chain, | GQGLEWMGRIDPEDGDSKYAPKFQGKATMTADTSTSTVYME | |
| IgG1KO) | LSSLRSEDTAVYYCTTSYYVGTYGYWGQGTLVTVSSASTKG | |
| | PSVFPLAPSSKSTSGGTAALGCLVKDYFPEPVTVSWNSGAL | |
| | TSGVHTFPAVLQSSGLYSLSSVVTVPSSSLGTQTYICNVNH | |
| | KPSNTKVDKRVEPKSCDKTHTCPPCPAPEAAGGPSVFLFPP | |
| | KPKDTLMISRTPEVTCVVVDVSHEDPEVKFNWYVDGVEVHN | |
| | AKTKPREEQYNSTYRVVSVLTVLHQDWLNGKEYKCKVSNKA | |
| | LPAPIEKTISKAKGQPREPQVYTLPPSREEMTKNQVSLTCL | |
| | VKGFYPSDIAVEWESNGQPENNYKTTPPVLDSDGSFFLYSK | |
| | LTVDKSRWQQGNVESCSVMHEALHNHYTQKSLSLSPGK | |
| Antibody C | QVQLVQSGAEVKKPGASVKVSCTASGFNIKDYYVHWVKQAP | 38 |
| (Heavy Chain, | GQGLEWMGRIDPEDGDSKYAPKFQGKATMTADTSTSTVYME | |
| IgG1) | LSSLRSEDTAVYYCTTSYYVGTYGYWGQGTLVTVSSASTKG | |
| | PSVFPLAPSSKSTSGGTAALGCLVKDYFPEPVTVSWNSGAL | |
| | TSGVHTFPAVLQSSGLYSLSSVVTVPSSSLGTQTYICNVNH | |
| | KPSNTKVDKRVEPKSCDKTHTCPPCPAPELLGGPSVFLFPP | |
| | KPKDTLMISRTPEVTCVVVDVSHEDPEVKFNWYVDGVEVHN | |
| | AKTKPREEQYNSTYRVVSVLTVLHQDWLNGKEYKCKVSNKA | |
| | LPAPIEKTISKAKGQPREPQVYTLPPSREEMTKNQVSLTCL | |
| | VKGFYPSDIAVEWESNGQPENNYKTTPPVLDSDGSFFLYSK | |
| | LTVDKSRWQQGNVESCSVMHEALHNHYTQKSLSLSPGK | |
| Antibody C | QVQLVQSGAEVKKPGASVKVSCTASGFNIKDYYVHWVKQAP | 39 |
| (Heavy | GQGLEWMGRIDPEDGDSKYAPKFQGKATMTADTSTSTVYME | |
| Chain, IgG4 DM) | LSSLRSEDTAVYYCTTSYYVGTYGYWGQGTLVTVSSASTKG | |
| | PSVFPLAPCSRSTSESTAALGCLVKDYFPEPVTVSWNSGAL | |
| | TSGVHTFPAVLQSSGLYSLSSVVTVPSSSLGTKTYTCNVDH | |
| | KPSNTKVDKRVESKYGPPCPPCPAPEFEGGPSVFLEPPKPK | |
| | DTLMISRTPEVTCVVVDVSQEDPEVQFNWYVDGVEVHNAKT | |
| | KPREEQFNSTYRVVSVLTVLHQDWLNGKEYKCKVSNKGLPS | |
| | SIEKTISKAKGQPREPQVYTLPPSQEEMTKNQVSLTCLVKG | |
| | FYPSDIAVEWESNGQPENNYKTTPPVLDSDGSFFLYSRLTV | |
| | DKSRWQEGNVESCSVMHEALHNHYTQKSLSLSLGK | |
| Antibody C | QVQLVQSGAEVKKPGASVKVSCTASGFNIKDYYVHWVKQAP | 40 |
| (Heavy Chain, | GQGLEWMGRIDPEDGDSKYAPKFQGKATMTADTSTSTVYME | |
| IgG1KOb) | LSSLRSEDTAVYYCTTSYYVGTYGYWGQGTLVTVSSASTKG | |
| | PSVFPLAPSSKSTSGGTAALGCLVKDYFPEPVTVSWNSGAL | |
| | TSGVHTFPAVLQSSGLYSLSSVVTVPSSSLGTQTYICNVNH | |
| | KPSNTKVDKRVEPKSCDKTHTCPPCPAPEAAGGPSVFLFPP | |
| | KPKDTLMISRTPEVTCVVVDVSHEDPEVKFNWYVDGVEVHN | |
| | AKTKPREEQYNSTYRVVSVLTVLHQDWLNGKEYKCKVSNKA | |
| | LPAPIEKTISKAKGQPREPQVYTLPPSRDELTKNQVSLTCL | |
| | VKGFYPSDIAVEWESNGQPENNYKTTPPVLDSDGSFFLYSK | |
| | LTVDKSRWQQGNVESCSVMHEALHNHYTQKSLSLSPGK | |

The variable regions were subcloned into one or two different suitable IgG expression vectors:
A) a human IgG1-KO (knock-out)/kappa format with a Leu234Ala, Leu235Ala double mutation in the Fc region to reduce effector function such as FcγR and complement binding
B) a human IgG4-DM (double mutant)/kappa format with a Ser228Pro mutation in the hinge region to reduce the occurrence of IgG4 half-molecules and a Leu235Glu mutation to further reduce FcγR binding

The Antibody A and Antibody B were purified and evaluated by the following criteria:
Appearance of CCF (turbidity)
Filtration properties of CCF
Yield on rProteinA
Turbidity upon elution and neutralization
Soluble aggregates (SEC)
Purity/contamination pattern (SDS)
Charge pattern (IEF)

### Example 2: Safety, Pharmacokinetics and Pharmacodynamics of BI 655064 in Phase 1 Clinical Trials in Healthy Chinese and Japanese Subjects

### Abstract

Aims: To evaluate the safety, pharmacokinetics and pharmacodynamics of BI 655064 in healthy Chinese and Japanese subjects after administration of single doses of 80-240 mg and multiple dosing of 240 mg once weekly over 4 weeks.

Methods: Two Phase 1, double-blind, placebo-controlled studies were conducted (single-rising doses of BI 655064 in Chinese/Japanese male subjects [n=12 per BI 655064 dose group] or repeated 240 mg BI 655064 in Chinese male subjects [n=9]). Plasma samples were collected to investigate BI 655064 pharmacokinetics, pharmacodynamics (CD40 receptor occupancy [RO]) and immunogenicity, along with safety and tolerability of BI 655064.

Results: BI 655064 showed good overall tolerability following single-dose administration of 80-240 mg and repeated administration of 240 mg BI 655064 over 4 weeks. More Chinese subjects reported adverse events compared with Japanese subjects following single-dose administration (59.4% vs 3.1%). BI 655064 exhibited non-linear, saturable kinetics, with higher doses resulting in slower apparent clearance (0.514-0.713 mL min 1), and disproportionately higher total exposure (AUC0-inf; 5610-7780 μg·h mL−1) and maximum plasma concentration (15,700-21,300 ng mL−1) with 240 mg BI 655064. Ninety percent inhibition of CD40 RO was achieved with doses 120 mg, and a direct relationship between BI 655064 plasma concentration and inhibition of CD40 RO was observed. Most subjects had a positive treatment emergent anti-drug antibody response.

Conclusions: BI 655064 pharmacokinetic and safety profiles in East Asian male subjects were consistent with those observed in a Western population. No adjustments in the BI 655064 dosing recommendations are warranted for future clinical trials.

### Introduction

The interaction of the cell surface receptor CD40 and its ligand CD40L (CD154) play an important role in the regulation of humoral and cellular immunity and in the pathology of autoimmune diseases such as rheumatoid arthritis (RA), systemic lupus erythematosus (SLE) and lupus nephritis (LN). SLE is a systemic autoimmune disease characterised by loss of B-cell tolerance to various autoantigens, particularly nucleic acids and their binding proteins. These autoantibodies form immune complexes that deposit in various tissues of the body and drive recruitment of inflammatory cells and mediators to the kidneys, leading to LN. Renal involvement in SLE varies with ethnicity, with East Asian patients with SLE exhibiting high rates of renal involvement (50-60%) compared with Caucasians (30-38%), with the highest rates of LN observed in Thailand and Sri Lanka (70-100%).

BI 655064 is a humanized, non-depleting, antagonistic therapeutic antibody that selectively binds human CD40 and blocks the CD40-CD40L interaction. Anti-CD40L antibodies lacking a functional fragment crystallisable (Fc) region are not associated with thromboembolic events. Two mutations (Leu234Ala and Leu235Ala) were introduced into the Fc region of BI 655064 to prevent Fc-mediated complement mediated cellular cytotoxicity and platelet activation. In patients with active RA, BI 655064 has been associated with reductions in inflammatory and bone resorption markers (IL-6, MMP-3 and receptor activator of nuclear factor κB ligand), concentrations of autoantibodies (IgG, IgM and IgA rheumatoid factors) and CD95+ activated B-cell subsets. The efficacy and safety of BI 655064 in patients with LN is currently being assessed in ongoing induction and maintenance studies.

BI 655064 has been administered as single-rising doses (SRDs) and multiple-rising doses (MRDs) to Western healthy volunteers. In the SRD study, BI 655064 was administered as intravenous (i.v.) doses between 0.2 and 120 mg and subcutaneous (s.c.) doses between 40 and 120 mg. BI 655064 exposure increased supra-proportionally to dose, with a terminal half-life between 4 h and 4 days i.v. and approximately 5 days s.c. Dose-related increases in the inhibition of CD40 receptor occupancy (RO) and CD54 upregulation after both i.v. and s.c. BI 655064 dosing were observed, and increasing single i.v. and s.c. doses up to 120 mg BI 655064; doses≥20 mg i.v. and 120 mg s.c. showed >90% CD40 receptor occupancy and inhibition of CD54 upregulation, which lasted 7 days in the 120 mg i.v. and s.c. group. All doses of BI 655064 were well tolerated.

In the MRD study, BI 655064 plasma concentrations increased supra-proportionally to dose, most probably due to target mediated clearance for doses between 80 and 120 mg, but was near proportional for doses >120 mg. The terminal half-life ranged between 6 and 8 days. Following 4 weeks of dosing, >90% CD40 receptor occupancy and inhibition of CD54 upregulation were observed at all dose levels, lasting for 17 days after the last dose. Ascending multiple s.c. doses of 80-240 mg BI 655064 were generally well tolerated, and no relevant signs of acute immune reaction were observed.

Here, we present the results from two studies conducted to characterise the safety, pharmacokinetics (PK) and pharmacodynamics (PD) of BI 655064 in healthy Chinese and Japanese Subjects subjects after administration of SRD (80-240 mg) and multiple-dosing of 240 mg BI 655064 once weekly (q1w) over 4 weeks.

### Methods

### Subjects

Eligible subjects were healthy East Asian male subjects of Chinese ethnicity (ethnic Chinese; born in China; or ethnic Chinese born outside of China and a descendent of four ethnic Chinese grandparents who were all born in China) or Japanese ethnicity (born in Japan, lived outside of Japan for <10 years and parents and grandparents who were all born in Japan), aged between 20 and 45 years, with a body mass index (BMI) ≥18.5 and ≤25 kg m-2.

### Study Designs

Study 1 was a randomized, double-blind, placebo-controlled within dose group, SRD study in Chinese and Japanese healthy male subjects (ClinicalTrials.gov identifier: NCT01917916). Subjects were randomized in a 3:1 ratio (BI 655064:placebo) to four sequential dose groups; 16 subjects (8 Chinese, 8 Japanese) per 80, 120, 180 and 240 mg BI 655064 dose group. Within each dose group, 12 subjects (6 Chinese, 6 Japanese) received BI 655064 and 4 subjects (2 Chinese, 2 Japanese) received placebo. Safety data were reviewed after each dose escalation. The doses of 80-240 mg BI 655064 used in study 1 were selected based on the PK/PD modelling from Caucasian healthy volunteers, demonstrating >90% inhibition of CD40 RO with weekly 80 mg BI 655064 dosing.

Study 2 was a randomized, double-blind, placebo-controlled, multiple-dose study in Chinese healthy male subjects (ClinicalTrials.gov identifier: NCT02331277). Subjects were randomised in a 3:1 ratio to receive 240 mg BI 655064 (9 subjects) or placebo (3 subjects) q1w over 4 weeks. The 240 mg dose was selected based on safety, PK and PD data from previous clinical studies in healthy volunteers. BI 655064 was administered as s.c. injections in both studies.

The objectives of these studies were to investigate the safety, tolerability, PK and PD of BI 655064 in healthy Chinese and Japanese male subjects following s.c. injection of SRDs of 80-240 mg and in healthy Chinese male subjects following multiple-doses of BI 655064 (q1w s.c. injections of 240 mg BI 655064 over 4 weeks).

These studies were conducted at the Seoul National University Hospital Clinical Trial Centre, Seoul, Korea, for subjects of Chinese ethnicity and at the Medical Co. LTA Sumida Hospital, Tokyo, Japan, for subjects of Japanese ethnicity. All subjects submitted written informed consent. Studies were conducted in accordance with the Seoul National University Hospital Institutional Review Board (Chinese ethnicity), Kyushu Clinical Pharmacology Research Institutional Review Board (Japanese ethnicity), Good Clinical Practice and the Declaration of Helsinki and its amendments.

### Safety Assessments

Safety was assessed by monitoring treatment-emergent adverse events (AEs; using MedDRA terms), physical examinations, vital signs, 12-lead electrocardiogram (ECG) and clinical laboratory tests (haematology, coagulation including bleeding time, clinical chemistry and urinalysis).

Tolerability was judged by the investigator according to the presence or absence of ‘swelling’, ‘induration’, ‘heat’, ‘redness’, ‘pain’ or ‘other findings’.

### Pharmacokinetic Assessments

Blood samples for PK analysis (2 mL) were collected from a forearm vein using an indwelling catheter into tripotassium ethylenediaminetetraacetic acid (K3 EDTA) anticoagulant tubes. For study 1, blood samples were collected pre-dose and at regular intervals up to 1656-hours post-dose. For study 2, blood samples were collected prior to the first dose, at regular intervals up to 144-hours post-first dose, prior to the second, third and fourth doses (at 168-hours, 336-hours and 504-hours post the first dose, respectively), and at regular intervals up to 3192-hours post the first dose.

Blood samples were immediately placed on ice after collection and centrifuged (2000-4000×g) at 4-8° C. for 10 minutes within 30 minutes of sample collection. Plasma was transferred into two polypropylene sample vials (approximately 0.4 mL each) and stored at ≤−20° C. until shipment to the analytical laboratory.

Plasma concentrations of BI 655064 were assessed at all visits using a validated sandwich enzyme-linked immunosorbent assay (ELISA; Covance Laboratories Inc., Chantilly, VA, USA) with a lower limit of quantification of 30 ng mL−1. The ELISA was developed and validated for the quantification of BI 655064 in human plasma. The method met all prospective criteria for system suitability, accuracy, precision, limits of quantitation, selectivity, dilutional linearity and analyte stability. Accuracy and precision were tested in six analytical runs and all levels had a total error (absolute % RE plus % CV) of less than 30%. The intra- and inter-assay accuracy and precision acceptance criteria of ±20%; ±25% at lower limit of quantitation (LLOQ) and (upper limited of quantitation (ULOQ) were met for this method. The quantitative range is from 30 to 800 ng/mL. Dilution linearity was established to 1/50,000. Selectivity, based on recovery of BI 655064 in human plasma, was acceptable in normal, haemolyzed and lipemic samples. Stability evaluations indicated that BI 655064 spiked in human plasma was stable after 6 freeze-thaw cycles, for approximately 24 hours at ambient room temperature, for 72 hours at 2 to 8° C., and up to 20 months at −60 to −80° C. and 12 months at −15 to −30° C. BI 655064 was stable in whole blood for up to four hours. Complete assay details are provided by Schwabe et al.

Plasma BI 655064 concentration-time data were analysed by a non-compartmental approach using Phoenix® WinNonlin® software (version 6.3, Certara L.P., Princeton, NJ 08540, USA). Parameters included maximum plasma concentration (Cmax), minimum plasma concentration (Cmin), time to achieve Cmax (tmax) and terminal half-life (t½) using the standard WinNonlin procedure. Area under the concentration-time curve over time zero to the last quantifiable plasma concentration (AUC0-tz) and AUC over the uniform dosing interval _{T} (AUC_{T}) were calculated using the WinNonlin linear up/log down algorithm. In study 1, the apparent clearance (CL/F) was calculated as dose/AUC0-inf, where F is the systemic availability and AUC0-inf is the AUC over the dose interval from time 0 extrapolated to infinity. In study 1, the apparent volume of distribution (Vz/F) was determined as (CL/F)/terminal elimination constant (λz). In study 2, the accumulation ratios (RA,Cmax based on Cmax; RA,AUC based on AUC_{T}) were calculated as the ratio of the value after the fourth dose to the value after the first dose.

### Pharmacodynamic Assessments

Blood samples for the determination of CD40 RO (2.7 mL) were collected from a forearm vein in a heparin anticoagulant tube. Afterwards, 1 mL of whole blood was transferred into a TransFix stabilisation tube and sent on ice to Boehringer Ingelheim Pharma GmbH & Co. KG, Germany for further analysis. Blood samples were collected pre-dose and at regular intervals up to 1320-hours post-dose for study 1, and prior to the first dose, at 72-hours post the first dose, prior to the second and fourth doses and at regular intervals up to 3192-hours post the first dose for study 2.

CD40 RO was analysed using a validated fluorescence activated cell sorting (FACS) assay, as described by Albach et al. CD40 RO was calculated using the ratio of observed fluorescence values from samples incubated with and without fluorescein isothiocyanate-labelled BI 655064). Inhibition of CD40 RO was expressed as a percentage and was calculated by putting the CD40 RO values from post dose measurements in relation to the respective pre-dose baseline value for each individual subject.

### Immunogenicity Assessments

Blood samples for measurement of antibodies against BI 655064 (anti-drug antibodies [ADAs]) (2 mL) were collected from a forearm vein in a K3-EDTA anticoagulant tube. Blood samples were collected pre-dose and at 264-, 984- and 1656-hours post-dose for study 1, prior to the first and fourth doses and at 912-, 1848-, 2520-, 3192- and 5880 hours post the first dose (follow-up visit) for study 2.

Blood samples were immediately placed on ice after collection and centrifuged (2000-4000×g) at 4-8° C. for 10 minutes within 30 minutes of sample collection. Plasma was transferred into two cryotubes (approximately 0.4 mL each) and stored at ≤−20° C. until shipment to the analytical laboratory.

Anti-drug antibodies to BI 655064 were analysed in plasma samples using a validated bridging assay (Covance Laboratories Inc., Chantilly, VA, USA). An electrochemiluminescence assay (ECL) assay using biotin- and ruthenium-labelled BI 655064 was validated with normal human plasma for the detection of anti-BI 655064 antibodies. For the confirmatory tier, the confirmatory cutpoint in healthy plasma was determined to be 35.7% inhibition in the presence of exogenously added BI 655064. Precision of the method was determined by the positive control to have CV<10%. BI 655064 drug tolerance for the anti-BI 655064 at a positive control antibody level of 250 ng/mL is 50 μg/mL.

A sample was considered ADA positive if its response in the screening assay was greater than or equal to a plate specific cut point and if it was confirmed positive in a specificity test (response blocked by the addition of BI 655064); confirmed ADA-positive samples were further characterised in a titre assay. Titres were determined by analysing serial two-fold sample dilutions. The reported titre was the highest-fold dilution that produced a mean electrochemiluminescent value greater than or equal to the confirmatory cut point. Complete assay details are provided by Schwabe et al. [11].

### Statistical Analysis

Study results were analysed using descriptive statistics for safety, PK and PD. No formal calculation of sample size was performed. The safety population included all subjects who had received BI 655064. The PK and PD populations included all subjects who had received BI 655064 and provided evaluable data for PK and PD analysis without relevant important protocol violations. A power model was used to explore the dose proportionality of Cmax, AUC0-tz and AUC0-inf in study 1. The model was defined as: exp(Yij)=α′*exp(Xi)β+ε′ij. After logarithmic transformations, the model was converted to the linear form: Yij=α+β*Xi+εij. Dose proportionality was assumed if the slope of the regression line (β) was equal to 1.

### Results

### Subjects

Demographic and baseline characteristics for studies 1 and 2 are summarised in Table 6. Study 1 enrolled 64 healthy male subjects (32 Chinese and 32 Japanese) and all subjects completed the study. Japanese subjects were slightly older than the Chinese subjects (mean age 28.5 years vs 25.2 years). Furthermore, Japanese subjects had a lower body weight and BMI compared with the Chinese subjects (overall mean body weight 63.9 kg vs 69.4 kg and overall mean BMI 21.3 kg m-2 vs 23.0 kg m-2, respectively). None of the Japanese subjects were smokers, whereas 17 of the 32 Chinese subjects were smokers.

TABLE 6
Demographics and baseline characteristics

| | Study 1 | | | | | | | |
|---|---|---|---|---|---|---|---|---|
| | BI 655064 | | | | | | | |
| | 80 mg | | 120 mg | | 180 mg | | 240 mg | |
| | CHI | JPN | CHI | JPN | CHI | JPN | CHI | JPN |
| All | (n = 6) | (n = 6) | (n = 6) | (n = 6) | (n = 6) | (n = 6) | (n = 6) | (n = 6) |
| Mean age, | 26.0 | 29.0 | 25.5 | 23.7 | 25.2 | 28.2 | 23.2 | 36.2 |
| years | (3.0) | (9.3) | (2.7) | (2.3) | (5.8) | (6.4) | (0.8) | (10.3) |
| (SD) | | | | | | | | |
| Mean | 71.2 | 65.7 | 74.3 | 67.2 | 66.3 | 59.8 | 67.0 | 67.5 |
| weight, | (3.9) | (6.0) | (7.3) | (9.4) | (4.6) | (7.5) | (6.9) | (5.2) |
| kg (SD) | | | | | | | | |
| Mean BMI, | 23.6 | 21.4 | 23.5 | 22.3 | 22.7 | 20.9 | 22.5 | 22.0 |
| kg m⁻² | (0.9) | (1.8) | (1.4) | (1.7) | (0.7) | (2.7) | (2.1) | (1.6) |
| (SD) | | | | | | | | |
| Smoking | | | | | | | | |
| history, n (%) | | | | | | | | |
| Never | 3.0 | 4.0 | 2.0 | 6.0 | 1.0 | 5.0 | 3.0 | 4.0 |
| smoked | (50.0) | (66.7) | (33.3) | (100.0) | (16.7) | (83.3) | (50.0) | (66.7) |
| Ex-smoker | 0 | 2.0 | 0 | 0 | 2.0 | 1.0 | 1.0 | 2.0 |
| | | (33.3) | | | (33.3) | (16.7) | (16.7) | (33.3) |
| Current | 3.0 | 0 | 4.0 | 0 | 3.0 | 0 | 2.0 | 0 |
| smoker^{a} | (50.0) | | (66.7) | | (50.0) | | (33.3) | |
| Alcohol | | | | | | | | |
| history, n (%) | | | | | | | | |
| Non-drinker | 2.0 | 2.0 | 3.0 | 3.0 | 2.0 | 3.0 | 5.0 | 3.0 |
| | (33.3) | (33.3) | (50.0) | (50.0) | (33.3) | (50.0) | (83.3) | (50.0) |
| Drinker^{b} | 4.0 | 4.0 | 3.0 | 3.0 | 4.0 | 3.0 | 1.0 | 3.0 |
| | (66.7) | (66.7) | (50.0) | (50.0) | (66.7) | (50.0) | (16.7) | (50.0) |

| | Study 1 | | | | Study 2 | | |
|---|---|---|---|---|---|---|---|
| | Placebo | | Total | | BI 655064 | | |
| | CHI | JPN | CHI | JPN | 240 mg | Placebo | Total |
| All | (n = 8) | (n = 8) | (N = 32) | (N = 32) | (n = 9) | (n = 3) | (N = 12) |
| Mean age, | 25.9 | 26.3 | 25.2 | 28.5 | 25.1 | 29.0 | 26.1 |
| years | (4.1) | (7.1) | (3.6) | (8.2) | (3.4) | (4.4) | (3.9) |
| (SD) | | | | | | | |
| Mean | 68.5 | 60.6 | 69.4 | 63.9 | 63.7 | 68 | 64.8 |
| weight, | (9.5) | (5.9) | (7.1) | (7.2) | (10.1) | (4.6) | (9.0) |
| kg (SD) | | | | | | | |
| Mean BMI, | 22.8 | 20.3 | 23.0 | 21.3 | 22.1 | 21.8 | 22.0 |
| kg m⁻² | (2.2) | (1.0) | (1.6) | (1.8) | (2.5) | (1.3) | (2.2) |
| (SD) | | | | | | | |
| Smoking | | | | | | | |
| history, n (%) | | | | | | | |
| Never | 2.0 | 7.0 | 11.0 | 26.0 | 5.0 | 0 | 5.0 |
| smoked | (25.0) | (87.5) | (34.4) | (81.3) | (55.6) | | (41.7) |
| Ex-smoker | 1.0 | 1.0 | 4.0 | 6.0 | 2.0 | 1.0 | 3.0 |
| | (12.5) | (12.5) | (12.5) | (18.8) | (22.2) | (33.3) | (25.0) |
| Current | 5.0 | 0 | 17.0 | 0 | 2.0 | 2.0 | 4.0 |
| smoker^{a} | (62.5) | | (53.1) | | (22.2) | (66.7) | (33.3) |
| Alcohol | | | | | | | |
| history, n (%) | | | | | | | |
| Non-drinker | 1.0 | 5.0 | 13.0 | 16.0 | 4.0 | 3.0 | 7.0 |
| | (12.5) | (62.5) | (40.6) | (50.0) | (44.4) | (100.0) | (58.3) |
| Drinker^{b} | 7.0 | 3.0 | 19.0 | 16.0 | 5.0 | 0 | 5.0 |
| | (87.5) | (37.5) | (59.4) | (50.0) | (55.6) | | (41.7) |

Results displayed as mean (standard deviation) for age, weight, and BMI. Results displayed as number of subjects (percentage number of subjects) for smoking and alcohol history.
^{a}Did not smoke more than 10 cigarettes, 3 cigars or 3 pipes per day.
^{b}At a level that did not interfere with study participation.
BMI, body mass index;
CHI, Chinese subject;
JPN, Japanese subject;
SD, standard deviation.

There were no relevant differences in demographic characteristics between the different dose groups for Chinese and Japanese subjects, except that the Japanese subjects in the 240 mg dose group in study 1 were slightly older than the Chinese subjects (mean age 36.2 vs 23.2 years; Table 6).

Study 2 enrolled 12 healthy Chinese subjects-one subject withdrew consent after receiving all four doses of placebo and 11 subjects completed the study.

There were no relevant differences in demographic characteristics between the treatment groups in study 2, except that the placebo group only contained subjects who were current or ex-smokers and none of the subjects in the placebo group drank alcohol (Table 6).

### Safety

Single-rising doses of BI 655064 up to 240 mg were well tolerated by Chinese and Japanese subjects, and multiple dosing of 240 mg BI 655064 q1w over 4 weeks was well tolerated by Chinese subjects. There were no serious AEs or AEs leading to discontinuation reported in either of these two studies, and all AEs were mild or moderate in severity and resolved by the end of study (Table 7). In study 1, only subjects of Chinese ethnicity experienced treatment-related AEs, and more treatment-related AEs were observed following single-dose administration of BI 655064 than placebo. The most frequently reported AE considered as drug-related was diarrhoea, which was reported by two of the 24 Chinese subjects (8.3%) in the pooled BI 655064 dose group compared with none of the 8 Chinese subjects (0%) treated with placebo in study 1. For Study 2, the only AEs that were considered as drug-related and that were reported for more than one subject were chest pain, headache, arthralgia, pain in extremity, and acne; of these, headache was the only event that was reported for a higher percentage of subjects in the BI 655064 group than in the placebo group (2 subjects [22.2%] on BI 655064 versus 0 subjects on placebo).

TABLE 7
Summary of AEs and frequency of treatment-related AEs

| | Study 1 | | | | | | Study 2 | |
|---|---|---|---|---|---|---|---|---|
| | | | | | | Total | BI | |
| | BI 655064 | | | | | BI | 655064 | |
| | 80 mg | 120 mg | 180 mg | 240 mg | Placebo | 655064 | 240 mg | Placebo |
| n (%) | (n = 12) | (n = 12) | (n = 12) | (n = 12) | (n = 16) | (N = 48) | (n = 9) | (n = 3) |
| Any AEs | 3 | 3 | 5 | 4 | 5 | 15 | 6 | 3 |
| | (25.0) | (25.0) | (41.7) | (33.3) | (31.3)^{a} | (31.3) | (66.7) | (100.0) |
| Any serious AEs^{b} | 0 | 0 | 0 | 0 | 0 | 0 | 0 | 0 |
| Any severe AEs^{c} | 0 | 0 | 0 | 0 | 0 | 0 | 0 | 0 |
| Infections | 2 | 0 | 1 | 1 | 2 | 4 | 2 | 1 |
| | (16.7) | | (8.3) | (8.3) | (12.5) | (8.3) | (22.2) | (33.3) |
| Upper respiratory | 2 | 0 | 1 | 1 | 2 | 4 | 2 | 1 |
| tract | (16.7) | | (8.3) | (8.3) | (12.4) | (8.3) | (22.2) | (33.3) |
| AEs leading to | 0 | 0 | 0 | 0 | 0 | 0 | 0 | 0 |
| discontinuation | | | | | | | | |
| Any treatment- | 2 | 1 | 4 | 2 | 1 | 9 | 5 | 2 |
| related AE^{d} | (16.7) | (8.3) | (33.3) | (16.7) | (6.3) | (18.8) | (55.6) | (66.7) |
| Most common | | | | | | | | |
| treatment-related | | | | | | | | |
| AEs^{e} | | | | | | | | |
| Diarrhoea | 1 | 0 | 0 | 1 | 0 | 2 | 0 | 0 |
| | (8.3) | | | (8.3) | | (4.2) | | |
| Chest pain | 0 | 0 | 0 | 0 | 0 | 0 | 2 | 1 |
| | | | | | | | (22.2) | (33.3) |
| Headache | 0 | 0 | 0 | 0 | 0 | 0 | 2 | 0 |
| | | | | | | | (22.2) | |

^{a}Included one subject of Japanese ethnicity (all other subjects with AEs were of Chinese ethnicity).
^{b}Defined as an AE that resulted in death, was immediately life-threatening, resulted in persistent or significant disability or incapacity, required or prolonged subject hospitalisation, was a congenital anomaly or birth defect, cancer or deemed serious for any other reason.
^{c}Defined as an AE that was incapacitating or caused an inability to work or perform usual activities.
^{d}Defined by the investigator.
^{e}AEs occurring in more than or equal to two subjects receiving BI 655064 are reported.
AE, adverse event.

No clinically relevant findings with respect to clinical laboratory tests (including bleeding times), vital signs, ECG, physical examinations or local tolerability were observed, except for one subject enrolled in the BI 655064 240 mg treatment group of study 2 who experienced pain after the third injection and ‘other findings’ (not specified further) after the fourth injection.

### Pharmacokinetics

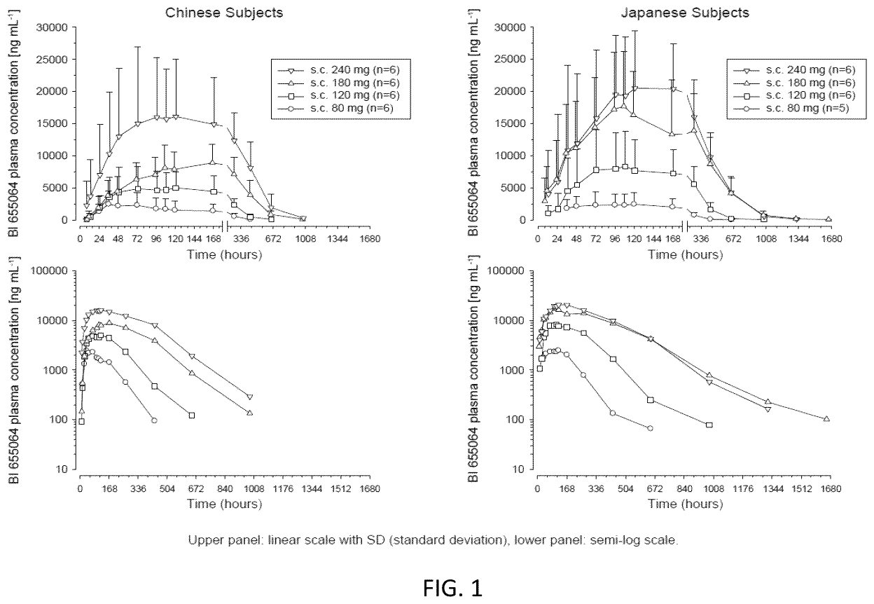

Following SRD administration in study 1, BI 655064 plasma concentrations increased with rising doses. The BI 655064 plasma concentration-time curves reached a peak at 96-144 hours post-dose, followed by at least a biphasic decline (FIG. 1). The terminal elimination half-life (t½) was generally long ranging, from 97.4-225 hours. Mean CL/F values were small (range: 0.467-4.04 mL min-1) and tended to decrease with increasing dose. Mean Vz/F values also decreased with increasing dose (range: 8.28-40.3 L). Coefficients of variation for Cmax and AUC parameters for the 120 mg-240 mg dose groups were typically within the range of 40-90%, suggesting moderate to high variability, and up to 1740% for the 80 mg dose group, suggesting very high variability (driven by one subject with very low AUC). Exposures (Cmax and AUCs) in Japanese subjects were generally higher than exposures in Chinese subjects in all dose groups; however, the exposure ratios (Japanese/Chinese) were smaller in the highest (240 mg) dose group (Cmax: 1.36; AUC0-inf: 1.39). The t½ values in Japanese subjects was slightly longer than that in the Chinese subjects, while the tmax values showed no apparent difference (Table 8).

TABLE 8
Summary of selected BI 655064 pharmacokinetic parameters following single-
dose administration to Chinese and Japanese subjects in study 1.

| | BI 655064 | | | | | | | |
|---|---|---|---|---|---|---|---|---|
| | 80 mg | | 120 mg | | 180 mg | | 240 mg | |
| Parameter | Chinese | Japanese | Chinese | Japanese | Chinese | Japanese | Chinese | Japanese |
| (unit) | (n = 6) | (n = 5) | (n = 6) | (n = 6) | (n = 6) | (n = 6) | (n = 6) | (n = 6) |
| Cₘₐₓ (ng | 888 | 1550 | 5160 | 7210 | 8650 | 16300 | 15700 | 21300 |
| mL⁻¹) | (501) | (315) | (51.6) | (92.8) | (41.1) | (73.6) | (54.2) | (53.4) |
| C_{max, norm} | 11.1 | 19.4 | 43.0 | 60.0 | 48.1 | 90.8 | 65.4 | 88.8 |
| (ng mL⁻¹ | (501) | (315) | (51.6) | (92.8) | (41.1) | (73.6) | (54.2) | (53.4) |
| mg⁻¹) | | | | | | | | |
| AUC_{0-tz} | 126 | 365 | 1110 | 2010 | 2900 | 6380 | 5680 | 7750 |
| (μg · h mL⁻¹) | (1740) | (165) | (47.2) | (79.8) | (60.4) | (84.6) | (52.4) | (57.2) |
| AUC_{0-tz, norm} | 1.58 | 4.56 | 9.23 | 16.7 | 16.1 | 35.4 | 23.7 | 32.3 |
| (μg · h mL⁻¹ mg⁻¹) | (1740) | (165) | (47.2) | (79.8) | (60.4) | (84.6) | (52.4) | (57.2) |
| AUC_{0-inf} | 330 | 464 | 1120 | 2020 | 2910 | 6430 | 5610 | 7780 |
| (μg · h mL⁻¹) | (111)^{a} | (74.1) | (46.6) | (78.8) | (60.1) | (83.3) | (59.0)^{a} | (57.1) |
| AUC_{0-inf, norm} | 4.12 | 5.80 | 9.34 | 16.9 | 16.2 | 35.7 | 23.4 | 32.4 |
| (μg · h mL⁻¹ mg⁻¹) | (111)^{a} | (74.1) | (46.6) | (78.8) | (60.1) | (83.3) | (59.0)^{a} | (57.1) |
| t_{1/2} (h) | 102 | 162 | 97.4 | 178 | 168 | 225 | 140 | 186 |
| | (54.2)^{a} | (157) | (42.2) | (45.0) | (55.4) | (62.8) | (54.6)^{a} | (42.1) |
| CL/F (mL | 4.04 | 2.87 | 1.78 | 0.989 | 1.03 | 0.467 | 0.713 | 0.514 |
| min⁻¹) | (111)^{a} | (74.1) | (46.6) | (78.8) | (60.1) | (83.3) | (59.0)^{a} | (57.1) |
| Vz/F (L) | 35.9 | 40.3 | 15.0 | 15.2 | 15.0 | 9.11 | 8.66 | 8.28 |
| | (233)^{a} | (433) | (59.2) | (104) | (73.5) | (187) | (112)^{a} | (76.9) |
| tₘₐₓ (h) | 96.0 | 120 | 102 | 138 | 144 | 102 | 108 | 144 |
| | (48.0-168) | (72.0-168) | (48.0-120) | (72.0-264) | (108-172) | (72.0-120) | (72.0-168) | (36.0-168) |

Data shown as geometric means (geometric coefficient of variation %), except for tₘₐₓ, which is presented as median (range).
^{a}n = 5.
AUC_{0-inf}, area under the concentration-time curve from time zero extrapolated to infinity;
AUC_{0-inf, norm}, dose normalised AUC_{0-inf};
AUC_{0-tz}, area under the concentration-time curve over time zero to the last quantifiable plasma concentration;
AUC_{0-tz, norm}, dose normalised AUC_{0-tz};
CL/F, apparent clearance;
Cₘₐₓ, maximum plasma concentration;
C_{max, norm}, dose normalised Cₘₐₓ;
t_{1/2}; terminal half-life;
tₘₐₓ, time to achieve Cₘₐₓ;
Vz/F, apparent volume of distribution.

Dose proportionality in Chinese and Japanese subjects was analysed over the entire dose range (80-240 mg). BI 655064 showed a more than dose proportional increase in AUCs (slope β=2.6-3.4) and Cmax (slope β=2.3-2.5) over the entire dose range (80-240 mg). Visual inspection using dose-normalised exposure also supported the supra-proportional increase in BI 655064 exposure (data not shown).

When dose proportionality was evaluated over the dose range of 120-240 mg, slope β estimates remained >1.5, but the 95% confidence intervals included unity for Cmax (Chinese and Japanese subjects) and AUCs (Japanese subjects).

In study 2, one subject who received a single dose of 240 mg BI 655064 in study 1 (approximately 1.5 years prior to enrolling into study 2) developed ADAs. The titre value was significantly boosted from 16 (baseline) up to 65,536 (just before the fourth dose of 240 mg BI 655064 q1w). Preliminary investigations suggest that this subject's ADAs interfered with the bioanalytical measurement of BI 655064 in the plasma; therefore, data from this subject were excluded from the PK analyses presented here.

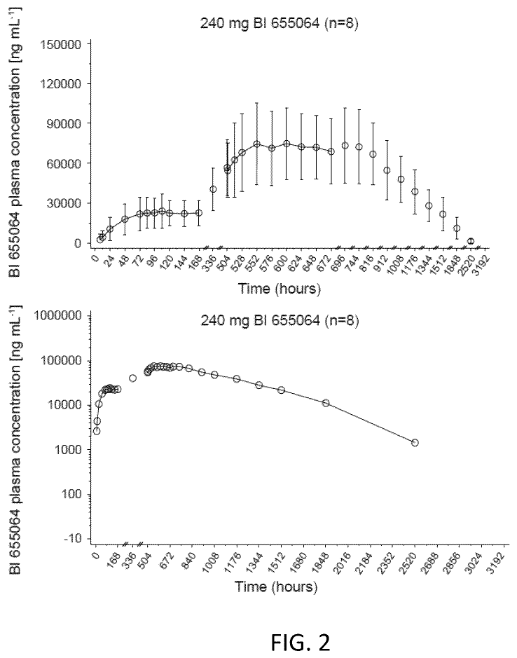

After doses of 240 mg BI 655064 q1w over 4 weeks in study 2, the plasma concentration-time curve reached a peak by 84.2 hours after the last dose followed by at least a biphasic decline with a t½ of 247 hours. Plasma concentrations did not reach steady state after the fourth dose (FIG. 2). The accumulation ratios based on Cmax and AUC_{T} after the fourth dose were 3.24 and 4.19, respectively (Table 9).

TABLE 9
Summary of selected BI 655064 pharmacokinetic parameters following
multiple-dose administration to Chinese subjects in study 2.

| | | BI 655064 | |
|---|---|---|---|
| | | 240 mg q1 w | |
| Dose | Parameter (unit) | n = 8 | |
| First Dose | C_{max, 1} (μg mL⁻¹) | 22.9 | (72.3) |
| | AUC_{t, 1} (μg · h mL⁻¹)^{a} | 2610 | (79.0) |
| | t_{max, 1} (h) | 132 | (72.0-168) |
| Fourth Dose | C_{max, 4} (μg mL⁻¹) | 74.1 | (50.4) |
| | C_{min, 4}(μg mL⁻¹) | 49.0 | (53.7) |
| | AUC_{t, 4} (μg · h mL⁻¹)^{a} | 10900 | (49.9) |
| | t_{1/2, 4} (h) | 247 | (39.8) |
| | t_{max, 4} (h) | 84.2 | (12.0-144) |
| | R_{A, AUC, 4} | 4.19 | (32.9) |
| | R_{A, Cmax, 4} | 3.24 | (24.8) |

Data shown as geometric means (geometric coefficient of variation %), except for tₘₐₓ, which is presented as median (range).
^{a}AUC_{T} is synonymous with AUC_{0-168 h}.
AUC_{T, 1}, area under the plasma concentration-time curve over a uniform dosing interval after the first dose; AUC_{T, 4}, area under the plasma concentration-time curve over a uniform dosing interval after the fourth dose; C_{max, 1}, maximum observed concentration after the first dose_{;} C_{max, 4}, maximum observed concentration after the fourth dose_{;} C_{min, 4}, minimum measured concentration of the analyte in plasma after the fourth dose; q1w, once weekly; R_{A, AUC, 4}, is equal to AUC_{T} after the fourth dose divided by AUC_{T} after the first dose; R_{A, Cmax, 4}, is equal to Cₘₐₓ after the fourth dose divided by Cₘₐₓ after the first dose; t_{1/2, 4}, terminal elimination half-life after the fourth dose; tₘₐₓ, time to maximum observed concentration; t_{max, 4}, time to maximum observed concentration after the fourth dose.

### Immunogenicity

In study 1, positive ADA responses were detected in most subjects (45/48 subjects) after a single dose of BI 655064, with response onset times in the terminal elimination phase of BI 655064 (Day 42 or Day 70 post-dose) in most cases (Table 10).

TABLE 10
Summary of positive ADA response following single-dose administration
to Chinese and Japanese subjects in study 1.

| | BI 655064 | | | | | | | | | |
|---|---|---|---|---|---|---|---|---|---|---|
| | 80 mg | | 120 mg | | 180 mg | | 240 mg | | Placebo | |
| | (n = 12) | | (n = 12) | | (n = 12) | | (n = 12) | | (n = 16) | |
| | Number of | | Number of | | Number of | | Number of | | Number of | |
| | subjects | | subjects | | subjects | | subjects | | subjects | |
| | with | | with | | with | | with | | with | |
| | positive | Titre, | positive | Titre, | positive | Titre, | positive | Titre, | positive | Titre, |
| | ADA | median | ADA | median | ADA | median | ADA | median | ADA | median |
| Timepoint | response | (range) | response | (range) | response | (range) | response | (range) | response | (range) |
| Pre-dose | 0 | NC | 0 | NC | 0 | NC | 0 | NC | 0 | NC |
| Day 12 | 4 | 3 | 1 | 4 | 1 | 1 | 0 | NC | 0 | NC |
| (264 hours | | (2-40) | | (NC) | | (NC) | | | | |
| post-dose) | | | | | | | | | | |
| Day 42 | 10 | 18 | 12 | 6 | 7 | 20 | 8 | 18 | 0 | NC |
| (984 hours | | (4-800) | | (1-400) | | (4-160) | | (4-400) | | |
| post-dose) | | | | | | | | | | |
| Day 70 | 11 | 40 | 12 | 28 | 11 | 20 | 11 | 80 | 0 | NC |
| (1656 hours | | (8-400) | | (2-400) | | (2-400) | | (2-160) | | |
| post-dose) | | | | | | | | | | |

ADA, anti-drug antibody;
NC, not calculated.

Positive ADA responses were also detected after repeat dosing with 240 mg BI 655064 in study 2 (Table 11). By Day 245, all nine subjects enrolled into the BI 655064 treatment group had a positive ADA response (median onset time: 105 days post-first dose). ADA responses were designated as treatment-induced or treatment-boosted based on recommendations from the 2014 White Paper on immunogenicity reporting by Shankar et al. [13]. 7/9 subjects (77.8%) exhibited a treatment-induced ADA-positive response and 2/9 subjects (22.2%) with pre-existing ADA's exhibited a treatment boosted ADA-positive response. One subject with a baseline titre of 1 was classified as boosted based on an increased titre later in the study. The ADA response from the subject who also participated in study 1 was significantly boosted from 16 to 65,536.

TABLE 11
Summary of positive ADA response following multiple-
dose administration to Chinese subjects in study 2.

| | BI 655064 240 mg, q1 w | | | Placebo | |
|---|---|---|---|---|---|
| | (n = 9) | | | (n = 3) | |
| | Number of | | | Number of | |
| | subjects with | | | subjects with | |
| | positive ADA | Titre, median | | positive ADA | Titre, median |
| Timepoint | response | (range) | | response | (range) |
| Pre-dose | 2 | 9 | (1-16) | 0 | NC |
| Day 21 (503.5 hours after | 1 | 65,536 | (NC) | 0 | NC |
| first dose) | | | | | |
| Day 38 (912 hours after | 1 | 32,768 | (NC) | 0 | NC |
| first dose) | | | | | |
| Day 77 (1848 hours after | 2^{a} | 8193 | (1-16,384) | 0 | NC |
| first dose) | | | | | |
| Day 105 (2520 hours after | 7 | 16 | (1-16,384) | 1 | 1 (NC) |
| first dose) | | | | | |
| Day 133 (3192 hours after | 8 | 24 | (4-8192) | 1 | 1 (NC) |
| first dose) | | | | | |
| Day 245 (5880 hours after | 9 | 16 | (16-4096) | 0^{b} | NC |
| first dose) | | | | | |

^{a}Only eight subjects were tested at this time point.
^{b}Only two subjects were tested at this time point.
ADA, anti-drug antibody; NC, not calculated; q1w, once weekly.

### Pharmacodynamics

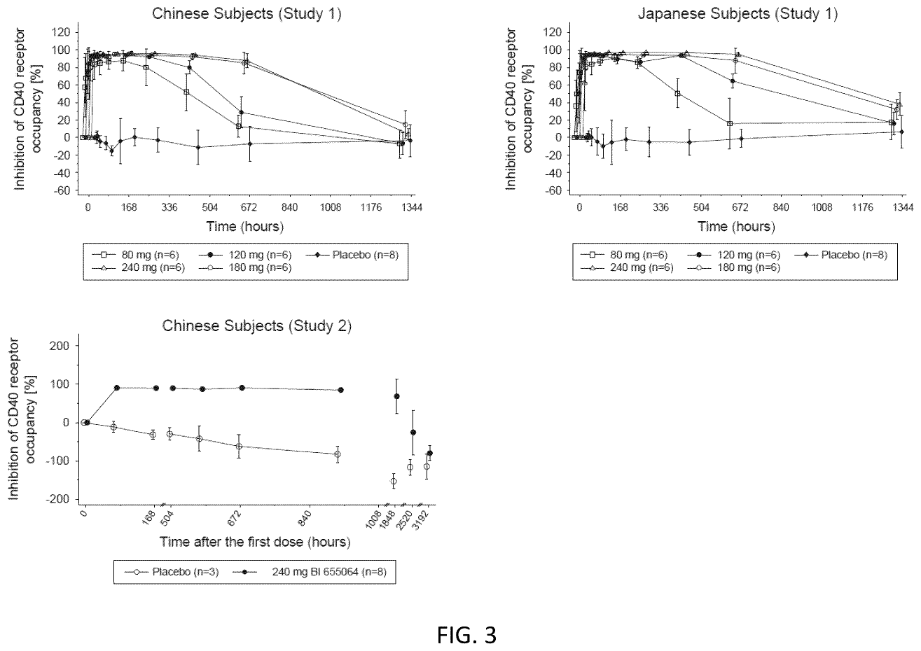

In the placebo dose group in study 1, no inhibition of CD40 RO was observed, while RO was inhibited in all active dose groups. Single s.c. administration of BI 655064 at doses ≥120 mg resulted in 90% inhibition of CD40 RO in Chinese subjects, and 90% inhibition of CD40 RO was achieved with all BI 655064 doses in Japanese subjects (FIG. 3). The duration of 90% inhibition increased with increasing BI 655064 dose (120 mg: 24-168 hours; 180 mg: 12-432 hours; 240 mg: 12-648 hours).

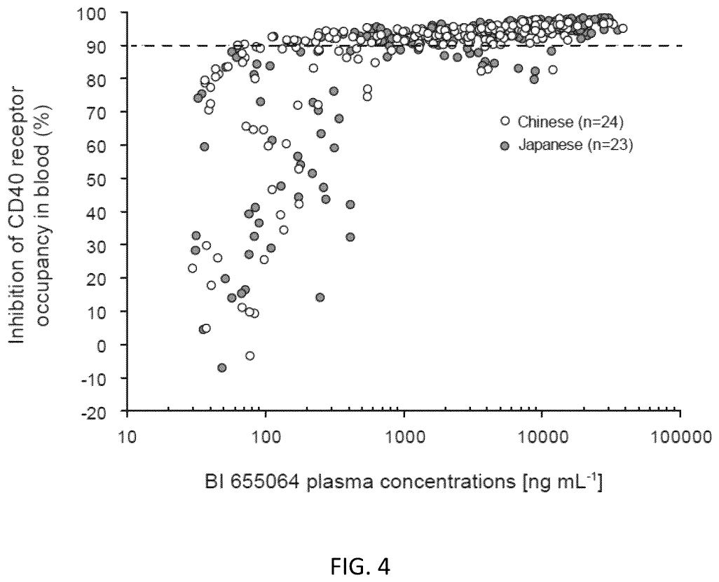

A direct relationship was observed between BI 655064 plasma concentrations and inhibition of CD40 RO in study 1. Ninety percent inhibition of CD40 RO was achieved with BI 655064 plasma concentrations ≥400 ng mL−1. Furthermore, there was no apparent difference in the relationship between BI 655064 plasma concentrations and inhibition of CD40 RO between Chinese and Japanese subjects (FIG. 4).

In study 2, the subject with a pre-existing ADA and boosted response, most probably due to participation in study 1, was excluded from this PD evaluation. Preliminary investigations suggest that ADAs may interfere with the results of the PD assay format, most probably due to the use of the FITC-labelled BI 655064 assay reagent. Following q1w s.c. administration of 240 mg BI 655064 over 4 weeks, 90% inhibition of CD40 RO was observed in the BI 655064 treatment group, with inhibition decreasing substantially between 1848 and 3192 hours after the first dose. In the placebo treatment group, inhibition of CD40 RO declined from baseline over time (FIG. 3).

### DISCUSSION

The two Phase 1 studies described here evaluated the safety, PK and PD of BI 655064 after administration of SRDs of 80-240 mg BI 655064 and multiple dosing with 240 mg BI 655064 q1w over 4 weeks in healthy East Asian subjects, and were conducted to support integration of East Asian subjects into a Phase 2 clinical trial.

BI 655064 showed good overall tolerability following administration of single s.c. doses of 80-240 mg and multiple dosing of 240 mg q1w over 4 weeks in healthy East Asian subjects. The observed AE profiles were consistent with results from similar studies conducted in a Western population. In general, the proportion of subjects with AEs were similar or lower than those observed in subjects who received placebo. Overall, a higher proportion of Chinese subjects reported any AE compared with Japanese subjects (59.4 vs 3.1%). The difference was observed for both subjects receiving BI 655064 as well as for subjects receiving placebo. The lower frequency of AEs reported in Japanese subjects has also been observed in other single- and multiple-dose studies reported in the literature comparing Japanese healthy volunteers with other ethnicities. However, the proportion of Chinese subjects reporting AEs was comparable to that observed in a similar SRD study performed in a Western population, where 41% of subjects reported AEs following BI 655064 s.c. or i.v. administration.

BI 655064 exhibited non-linear, saturable kinetics with higher doses of BI 655064, resulting in slower clearance and disproportionately higher plasma exposure (AUC and Cmax). Clearance reached a minimum of 0.514-0.713 mL min-1 at the 240 mg dose level. The observed BI 655064 PK profile in East Asian subjects is comparable to the profile observed in a Western population and is consistent with other compounds exhibiting non linear PK, possibly due to a target-mediated pathway.

The individual BI 655064 exposures for subjects were compared among Caucasian, Japanese and Chinese populations as shown in Supplemental FIG. 1; there was large variability within and across ethnicities in Cmax and AUC0-168 after single dose (Supplemental FIGS. 1A and 1B, respectively) and multiple doses (Supplemental FIGS. 1c and 1D, respectively) observed. Considering the large variability, there were no apparent differences in BI 655064 exposure (AUC, Cmax) following SRD administration over a 80-240 mg dose range or following multiple dosing in East Asian subjects to those observed in a Western population.

A trend towards decreasing BI 655064 exposure with increasing body weight and BMI was observed. BI 655064 exposure (AUC, Cmax) in Japanese subjects was generally higher than in Chinese subjects across the 80-240 mg dose range in study 1. This may be due to the correlation between BI 655064 exposure and body weight, given that Japanese subjects had a lower baseline body weight than the Chinese subjects enrolled into study 1 (Table 1). Since the number of subjects is limited per dose group, further investigation with a larger number of subjects is needed to show the effect of covariates quantitatively such as body weight or smoking history.

Ninety percent inhibition of CD40 RO was achieved with doses of BI 655064≥120 mg in this East Asian population, and a direct relationship between BI 655064 plasma concentration and inhibition of CD40 RO was observed. Higher doses of BI 655064 may be required to achieve >90% CD40 RO in patients with LN, as inflammation in LN is not only driven by B cells, but also monocytes, macrophages, dendritic cells and kidney resident cells (including mesangial cells, podocytes and proximal tubule cells), which all express CD40.

In study 2, the placebo group showed a decline in CD40 RO inhibition (from 0 at baseline to <−100% at the last sampling time point), which was associated with an unusual decline in the fluorescence intensities of the unstained samples. The decline occurred in both treatment groups and the reason for this is not known (no deviations observed in sample stability, assay conduct or sample integrity). Therefore, CD40 RO data from study 2 should be interpreted with caution.

Nearly all East Asian subjects enrolled into these studies had a positive, treatment emergent ADA response. While the incidence was high, the ADA responses were predominately observed at the end of the follow up periods in these studies, where BI 655064 plasma concentrations were near the lower limit of assay quantification and BI 655064 had largely been eliminated; therefore, the effect of ADAs on BI 655064 PK could not be evaluated. In previous studies conducted in a Western population, the ADA response was modest, with titres being relatively low and highly variable (2-640) [11]. Higher incidences of ADAs in East Asian subjects compared with Caucasian subjects have been observed with other monoclonal antibodies, including adalimumab and efalizumab.

In study 2, the ADA titres were relatively low (1-64), except for the subject who had previously received BI 655064 in study 1 and had a pre existing ADA response at baseline in study 2. This subject had a significantly boosted ADA response during study 2 but showed no safety findings related to the presence of ADAs. A neutralising ADA assay is not currently available; therefore, no conclusion could be drawn about whether ADAs were neutralising or not. Boosted ADA responses have also been reported following re exposure with the humanized anti-CD52 monoclonal antibody alemtuzumab.

In these studies, an impact of ADA on PK and the PD assays cannot be excluded. The cause for the high incidence of ADA responses observed is not known, but could be due to the expression of CD40 on dendritic cells. In a recent clinical trial with BI 655064 in patients with RA with an inadequate response to methotrexate, the incidence of ADAs was low (6/44 patients) and all ADA titres were ≤8.

In conclusion, the BI 655064 PK and safety profiles in East Asian male subjects were consistent with those observed in previous studies conducted in a Western population; therefore, no adjustments to the BI 655064 dosing recommendations are warranted for future clinical studies. A high incidence of ADAs was observed in this East Asian population; therefore future clinical trials should monitor the immunogenicity profiles of patients co administering BI 655064 with other immunosuppressants (i.e. methotrexate), which may reduce the incidence of ADA responses, as seen with other biologics in RA.

### Example 3: Randomised Dose Ranging, Placebo-Controlled, Phase II Study Assessing the Efficacy and Safety of BI 655064, an Antagonistic Anti-CD40 Antibody, in Patients with Lupus Nephritis

Background: In patients (pts) with SLE, activation of the CD40-CD40L pathway results in stimulation and proliferation of B cells and other inflammatory cell types. The subsequent generation of autoantibodies and their deposition in the kidney, as well as activation of myeloid and resident kidney cells, result in local inflammation and eventually, kidney injury. Thus, CD40 is an appealing therapeutic target in lupus nephritis (LN). BI 655064 is a humanised anti-CD40 monoclonal antibody that blocks the CD40 pathway in a nanomolar range and downregulates activated B cells.

The purpose of this study was to test whether different doses of BI 655064 help people with active lupus nephritis. In particular, we assess the efficacy and safety over 52 weeks (wks) of three doses of subcutaneous BI 655064 compared with placebo, as add-on to mycophenolate and steroids, in patients with active proliferative LN (Lupos Nephritis).

### Objectives

The objectives were to characterise the dose-response relationship, identify the target dose for Phase III development, and investigate the safety and efficacy of 3 doses of BI 655064 administered subcutaneously for 52 weeks as add-on therapy to standard of care treatment in patients with active lupus nephritis.

### Study Design/Methodology

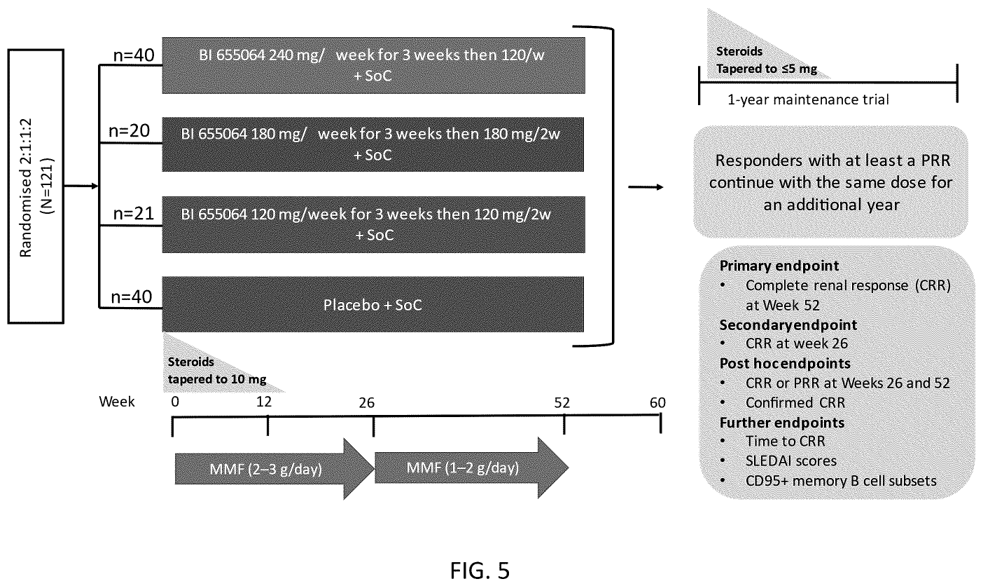

This multicentre, randomised, placebo-controlled, double-blind, parallel-group, Phase II trial in patients with active LN was conducted at 74 sites across 20 countries between 16 Aug. 2016 and 18 Aug. 2020. A total of 121 patients were randomised in a 2:1:1:2 ratio to placebo or BI 655064 120 mg, 180 mg or 240 mg; all patients received SoC (standard of care). (FIG. 5) Randomisation was stratified by race (Asian vs. non-Asian) and proteinuria at screening (urine protein/urine creatinine ratio [UP/UC]<3 or ≥3). The primary efficacy analysis was done after 52 weeks of treatment. Patients with at least a partial renal response or UP/UC<1 at Week 52 could decide if they wanted to participate in the maintenance trial 1293.13.

### Number of Patients

Planned Randomised: 120; Actual Randomized: 121.

BI 655064 120 mg: Randomised: 21; Treated: 21; Analysed: 21.

BI 655064 180 mg: Randomised: 20; Treated: 20; Analysed: 20.

BI 655064 240 mg: Randomised: 40; Treated: 40; Analysed: 40.

Placebo: Randomised: 40; Treated: 40; Analysed: 40.

Overall, 121 patients with LN were randomised, double blind, in a 2:1:1:2 ratio to placebo or BI 655064 120 mg, 180 mg or 240 mg, and received a weekly loading dose for the first 3 wks, followed by dosing every 2 wks for the 120 and 180 mg doses, and weekly (120 mg) for the 240 mg group. Key inclusion criteria included an active ISN/RPS class III or IV (±V) renal biopsy within 3 months prior to screening and a screening protein/creatinine ratio of 1 mg/mg. Randomisation was stratified based on race (Asian vs non-Asian) and screening protein/creatine (UP/UC) ratio (<3 vs ≥3). The primary efficacy endpoint was complete renal response (CRR), defined as 24 h proteinuria <0.5 g/day and stable eGFR at Wk 52.

SoC during the initial phase of the trial (Weeks 1-26) included: MMF at a dose of 2-3 g/day. A dose of <2 g/day was permitted if patients experienced MMF-related adverse events (AEs). High-dose steroids, which included pulsed methylprednisolone (500 mg intravenous [IV] for 3 days) followed by oral steroids tapered to prednisone-equivalent of 10 mg per day within 12 weeks of randomization. Patients who received ≤3 g IV steroids within the 6 weeks before randomisation only received the number of IV steroid pulses required to reach 1.5 g. A maximum dose of 1000 mg methylprednisolone per day for up to 3 days (≤3000 mg total) was permitted if deemed necessary by the investigator.

SoC during the second phase of the trial (Weeks 26-52) included MMF (1-2 g/day) in combination with 510 mg prednisone-equivalent per day. Patients who experienced MMF-related AEs were permitted a dose reduction to 1 g/day. Investigators were permitted to increase the steroid dose if patients experienced disease worsening or flares.

The study endpoints are described in FIG. 5. Complete renal response (CRR) was defined as urine protein (UP)<0.5 g/day and either normal estimated glomerular filtration rate (eGFR) or <20% decrease from baseline if eGFR was below the normal range. Partial renal response (PRR) was defined as 50% reduction in proteinuria from baseline and normal eGFR or <20% decrease in eGFR from baseline if eGFR was below normal range.

### Diagnosis

Patients with lupus nephritis (class III or IV according to the ISN/RPS 2003 classification).

### Main Criteria for Inclusion

Male and female patients, aged 18 to 70 years at Visit 1, with a diagnosis of systemic lupus erythematosus (SLE) by ACR criteria 1997. At least 4 criteria had to be documented, one of which had to be a positive anti dsDNA antibody OR a positive antinuclear antibody (ANA) at screening or around the time of start of induction therapy. Patients had to have lupus nephritis class III or IV (ISN/RPS 2003 classification) with either active or active/chronic disease, co-existing class V permitted, proven by renal biopsy within 3 months (6 months in the US) prior to screening or during screening in case induction therapy for lupus nephritis had not been started, as well as active renal disease evidenced by proteinuria ≥1.0 g/day (respectively UP/UC≥1).

Investigational product: B1655064; Dose: Dose group 1: 120 mg once a week for 3 weeks followed by 120 mg once every 2 weeks. Dose group 2: 180 mg once a week for 3 weeks followed by 180 mg once every 2 weeks. Dose group 3: 240 mg once a week for 3 weeks followed by 120 mg once a week. Mode of administration: Subcutaneous injection.

Comparator product: Placebo; Dose: not applicable; mode of administration: subcutaneouse injection.

Duration of Treatment: 52 weeks of treatment followed by a follow-up period of 8 weeks.

### Efficacy Criteria for Evaluation

The primary endpoint in this trial was the proportion of patients with complete renal response (CRR) at Week 52. The secondary endpoints were: Proportion of patients with CRR at Week 26; Proportion of patients with partial renal response (PRR) at Weeks 26 and 52; Proportion of patients with major renal response (MRR) at Weeks 26 and 52.

### Safety Criteria for Evaluation

Safety criteria included adverse events (AEs), safety laboratory tests, physical examinations (including weight measurement), vital signs (blood pressure, pulse rate), and 12-lead electrocardiograms.

### Statistical Methods

The statistical design for this Phase II dose finding trial was a multiple comparison procedure and modelling (MCPMod) approach. CRR at Week 52 (derived using urine protein [UP] and derived using UP/UC from 24 h urine collections) was analysed using a logistic regression model. Factors in the model included treatment and the covariates race (Asian/non-Asian) and proteinuria at screening (UP/UC<3 or ≥3). From this model, placebo-adjusted treatment estimates for each active dose group, as well as their corresponding variance-covariance matrix were estimated. These results were used in the MCPMod analysis. Pairwise comparisons of the modelled proportions of patients with CRR at each dose level to placebo were performed. Also, pairwise comparisons of the observed proportions of patients with CRR at each dose level to placebo were performed. Confidence intervals were calculated using the Newcombe method and p values from Barnard tests of association were derived.

An interim analysis was conducted after all patients had completed 26 weeks of treatment.

### Results:

The placebo response in this trial was higher than expected (48.3%; Table 12); none of the BI 655064 doses increased rates of CRR at Wk 52 compared with placebo. However, CRR at Wk 52 based on creatinine-adjusted proteinuria, assessed using spot urine, showed a better response in the 180 mg group (50%) vs placebo (42.5%), and the 180 mg dose showed a greater change from baseline over time vs placebo from Wk 4. Time to CRR was shorter in the 180 mg group (17.3 wks) vs placebo (20.4 wks). The 180 mg group also showed improvement vs placebo in total SLEDAI (SELENA) and its subscores.

TABLE 12
Efficacy endpoints at Wk 52

| | | BI 655064 | | |
|---|---|---|---|---|
| | Placebo | 120 mg | 180 mg | 240 mg |
| | (n = 40) | (n = 21) | (n = 20) | (n = 40) |
| Observed CRR, n | 20 | 8 | 9 | 18 |
| Adjusted* CRR, % | 48.3 | 38.3 | 45 | 44.6 |
| Observed cCRR, n | 13 | 5 | 9 | 16 |
| Adjusted* cCRR, % | 29.1 | 22.5 | 44.3 | 38.2 |
| Mean change from | | | | |
| baseline in SLEDAI | | | | |
| Total score | −6.5 | −6.1 | −9.7 | −8.2 |
| Non-renal score | −1.4 | −3.0 | −2.8 | −3.1 |
| Renal score | −5.1 | −3.7 | −6.8 | −5.0 |
| Clinical score | −5.7 | −3.9 | −7.9 | −6.5 |

Complete renal response (CRR) based on 24 h proteinuria; confirmed CRR (cCRR) based on UP/UC (spot urine) at Wks 46 and 52.
*Logistic regression model including treatment and the covariates race and proteinuria at screening.

The unexpected high placebo response prompted a post hoc analysis evaluating confirmed CRR (cCRR), whereby confirmation of the endpoint was required at both Wks 46 (penultimate visit on treatment) and 52. A 15.2% higher cCRR in the 180 mg group (44.3%) vs placebo (29.1%) was observed (p=0.26).

While based on a small sample size, there were more reports of infection-related severe and serious adverse events and neutropenia in the 240 mg group compared with placebo. Of note, in those who experienced neutropenia, a clinical impact (e.g. increase in infections) was not established. Aside from these observations, safety data were comparable across treatment groups.

Larger decreases from baseline were observed in the percentage of CD27−IgD−CD95+, CD27−IgD+CD95+, CD27+IgD+CD95+ and CD27+IgD−CD95+ B-cell subsets in the 180 and 240 mg groups compared with placebo.

Treatment-emergent anti-drug antibodies (ADAs) were detected in five pts treated with BI 655054, all at low titre, and in one who received placebo; ADAs had no impact on pharmacokinetics or safety.

### Summary—Conclusions

The trial did not meet its primary CRR endpoint. However, when confirmation of CRR was required at both Wks 46 and 52, the resultant decrease in the placebo response generated an effect size of 15.2% and 9.1% in favour of 180 mg and 240 mg BI 655064, respectively (Table 13).

TABLE 13
Efficacy endpoints at Wk 52

| | | BI 655064 | | |
|---|---|---|---|---|
| | Placebo | 120 mg | 180 mg | 240 mg |
| | (n = 40) | (n = 21) | (n = 20) | (n = 40) |
| Observed CRR, n | 20 | 8 | 9 | 18 |
| Adjusted* CRR, % | 48.3 | 38.3 | 45 | 44.6 |
| Observed cCRR, n | 13 | 5 | 9 | 16 |
| Adjusted* cCRR, % | 29.1 | 22.5 | 44.3 | 38.2 |
| Mean change from | | | | |
| baseline in SLEDAI | | | | |
| Total score | −6.5 | −6.1 | −9.7 | −8.2 |
| Non-renal score | −1.4 | −3.0 | −2.8 | −3.1 |
| Renal score | −5.1 | −3.7 | −6.8 | −5.0 |
| Clinical score | −5.7 | −3.9 | −7.9 | −6.5 |

CRR based on 24 h proteinuria; cCRR based on UP/UC (spot urine) at Wks 46 and 52.
*Logistic regression model including treatment and the covariates race and proteinuria at screening.

Trial Patients and Compliance with the Clinical Trial Protocol

A total of 209 patients were enrolled in this trial. Of these, 121 patients were randomised to 1 of 4 treatment groups (BI 655064 120 mg: 21 patients, BI 655064 180 mg: 20 patients, BI 655064 240 mg: 40 patients, placebo: 40 patients). All patients were treated with at least one dose of trial medication. The proportion of patients who prematurely discontinued trial medication was 33.3% in the 120 mg group, 15.0% in the 180 mg group, 12.5% in the 240 mg group, and 17.5% in the placebo group. The most common reason for premature discontinuation was AE (120 mg: 19.0%, 180 mg: 15.0%, 240 mg: 12.5%, placebo: 7.5%).

The vast majority of patients were female (89.3%); however, the proportion of women was lower in the 120 mg group (76.2%) and higher in the placebo group (95.0%) than in the 180 mg and the 240 mg groups (90.0% each). Overall, 52.1% of patients were White, 43.0% Asian, 3.3% Black or African American, 0.8% of multiple race, and for 0.8% the information on race was missing. The proportion of White patients was slightly lower in the 120 mg group than in the other treatment groups. About 20% of patients were of Hispanic ethnicity; however, the placebo group contained a slightly lower percentage of Hispanic patients than the other treatment groups. The mean (standard deviation [SD]) age of the patient population was 34.5 (10.2) years and the mean (SD) weight was 63.5 (15.2) kg.

There were a few imbalances between the treatment groups with respect to the proportion of patients with a time since diagnosis <6 months (120 mg: 28.6%, 180 mg: 30.0%, 240 mg: 42.5%, placebo: 47.5%), the proportion of patients with global class IV lupus nephritis (based on local renal pathologist's assessment; 120 mg: 47.6%, 180 mg: 40.0%, 240 mg: 40.0%, placebo: 32.5%), the proportion of patients with anti-dsDNA >ULN at baseline (120 mg: 52.4%, 180 mg: 30.0%, 240 mg: 67.5%, placebo: 55.0%), mean eGFR [mL/min/1.73 m2] at baseline (120 mg: 85.9, 180 mg: 99.9, 240 mg: 91.1, placebo: 88.8), and mean UP [g/day] at baseline (120 mg: 4.3, 180 mg: 3.0, 240 mg: 3.2, placebo: 2.8).

All patients used corticosteroids (predominantly methylprednisolone, prednisone, and prednisolone) and mycophenolate mofetil as standard of care therapy in this trial. Other frequently used therapies at system organ class level were antimalarials, drugs for peptic ulcer and gastro-oesophageal reflux disease, calcium, other analgesics and antipyretics, and angiotensin converting enzyme inhibitors (ACEi). The proportion of patients using ACEi or angiotensin receptor blocker therapies on treatment was higher in the placebo group (82.5%) than in the BI 655064 groups (120 mg: 61.9%, 180 mg: 70.0%, 240 mg: 77.5%).

Most trial patients had more than 48 weeks exposure to trial medication. The mean (SD) exposure was 41.6 (15.9) weeks in the 120 mg group, 49.0 (7.9) weeks in the 180 mg group, 48.7 (8.0) weeks in the 240 mg group, and 46.5 (12.6) weeks in the placebo group.

Important protocol deviations (iPDs) were reported for 12.4% of patients (120 mg: 19.0%, 180 mg: 0%, 240 mg: 15.0%, placebo: 12.5%). The most common iPDs were related to concomitant medication use.

### Efficacy Results

Primary Endpoint: Proportion of Patients with Complete Renal Response at Week 52.

The adjusted proportion of patients achieving CRR at Week 52 (based on UP from 24 h urine collections) was higher in the placebo group than in the BI 655064 treatment groups (Table 14). The placebo response rate observed at Week 52 was notably higher than expected. In the MCPMod analysis, all prespecified models showed a flat dose-response shape.

TABLE 14
Adjusted (model-based) proportion of patients with complete
renal response (based on UP 24 h) at Week 52

| | | | | Delta vs. | |
|---|---|---|---|---|---|
| | Patients | Patients with CRR | | placebo² | |
| | N | n | %¹ | % | p-value |
| Placebo | 40 | 20 | 48.3 | | |
| BI 655064 120 mg | 21 | 8 | 38.3 | −10.0 | 0.4645 |
| BI 655064 180 mg | 20 | 9 | 45.0 | −3.4 | 0.8084 |
| BI 655064 240 mg | 40 | 18 | 44.6 | −3.8 | 0.7398 |

N = number of patients in each group, n = observed number of patients with CRR
¹Adjusted proportion of patients with CRR
²The difference in probabilities was calculated by subtracting the corresponding probabilities.

The analysis of CRR using the alternate derivation based on UP/UC from the 24 h urine collections yielded similar results, while in an analysis of CRR using UP/UC from spot urines, the observed proportion of patients achieving CRR at Week 52 was higher in the 180 mg and 240 mg groups compared with placebo. (50.0% and 47.5% vs. 42.5%). In a post-hoc analysis of CRR based on UP/UC from spot urines at 2 time points (Week 46 and Week 52; ‘confirmed CRR’), the adjusted proportion of patients achieving CRR was notably higher in the 180 mg and 240 mg groups than in the placebo group (44.3% and 38.2% vs. 29.1%).

Secondary Endpoints: Proportion of Patients with Complete Renal Response at Week 26.

At Week 26, the 180 mg group was the only treatment group with a higher proportion of patients achieving CRR (based on UP 24 h) versus placebo. The observed proportions of patients with CRR at Week 26 were 37.5% (placebo), 28.6% (120 mg), 50.0% (180 mg), and 35.0% (240 mg). The analysis of CRR at Week 26 based on UP/UC 24 h yielded similar results.

Secondary Endpoint: Proportion of Patients with Partial Renal Response at Weeks 26 and 52.

At Week 26 and at Week 52, the 180 mg group was the only treatment group with a higher proportion of patients achieving PRR (based on UP 24 h) versus placebo. The observed proportions of patients with PRR at Week 26 were 62.5% (placebo), 42.9% (120 mg), 75.0% (180 mg), and 62.5% (240 mg). The observed proportions of patients with PRR at Week 52 were 60.0% (placebo), 33.3% (120 mg), 65.0% (180 mg), and 55.0% (240 mg). In the analysis of PRR at Week 52 based on UP/UC 24 h, the 180 mg and 240 mg treatment groups showed a higher proportion of patients achieving PRR versus placebo.

Secondary Endpoint: Proportion of Patients with Major Renal Response at Weeks 26 and 52.

At Week 26 and at Week 52, the 180 mg group was the only treatment group with a higher proportion of patients achieving MRR (based on UP 24 h) versus placebo. The observed proportions of patients with MRR at Week 26 were 50.0% (placebo), 28.6% (120 mg), 55.0% (180 mg), and 37.5% (240 mg). The observed proportions of patients with MRR at Week 52 were 52.5% (placebo), 42.9% (120 mg), 55.0% (180 mg), and 52.5% (240 mg). In the analysis of MRR at Week 52 based on UP/UC 24 h, the 240 mg group was the only treatment group demonstrating a higher proportion of patients achieving MRR versus placebo.

### Safety Results

Almost all patients were reported with at least 1 AE (adverse event). The most frequently reported AEs (>25% in any treatment group) by system organ class (SOC) were infections and infestations (120 mg: 61.9%, 180 mg: 75.0%, 240 mg: 75.0%, placebo: 60.0%), skin and subcutaneous tissue disorders (23.8%, 30.0%, 55.0%, 47.5%), gastrointestinal disorders (38.1%, 40.0%, 40.0%, 35.0%), musculoskeletal and connective tissue disorders (33.3%, 20.0%, 32.5%, 45.0%), general disorders and administration site conditions (38.1%, 15.0%, 25.0%, 30.0%), investigations (19.0%, 25.0%, 40.0%, 17.5%), nervous system disorders (14.3%, 20.0%, 30.0%, 30.0%), and blood and lymphatic system disorders (14.3%, 20.0%, 37.5%, 10.0%). The most frequently reported AEs (>15% in any treatment group) by preferred term (PT) were upper respiratory tract infection (23.8%, 35.0%, 17.5%, 20.0%), diarrhoea (23.8%, 15.0%, 22.5%, 15.0%), alopecia (0%, 15.0%, 22.5%, 17.5%), nasopharyngitis (9.5%, 5.0%, 10.0%, 17.5%), and neutropenia (4.8%, 15.0%, 17.5%, 2.5%).

Infections and infestations were the most frequent AEs leading to discontinuation; they were reported for 1 patient (5.0%) in the 180 mg group and 3 patients (7.5%) in the 240 mg group. Proteinuria was the only AE leading to discontinuation on PT level that was reported for >1 patient overall (1 patient on 240 mg and 1 patient on placebo).

Drug-related AEs (as defined by the investigator) most frequently belonged to the SOCs infections and infestations (120 mg: 28.6%, 180 mg: 15.0%, 240 mg: 40.0%, placebo: 22.5%), investigations (14.3%, 0%, 20.0%, 10.0%), blood and lymphatic system disorders (4.8%, 5.0%, 15.0%, 7.5%), and general disorders and administration site conditions (19.0%, 0%, 7.5%, 10.0%). The most common drug-related PTs (≥10% in any treatment group) were upper respiratory tract infection (4.8%, 10.0%, 12.5%, 10.0%), herpes zoster (4.8%, 0%, 10.0%, 2.5%), and neutropenia (0%, 5.0%, 10.0%, 2.5%).

AEs of maximum RCTC grade 3 were most frequently reported in the SOCs blood and lymphatic system disorders (120 mg: 4.8%, 180 mg: 5.0%, 240 mg: 12.5%, placebo: 5.0%) and infections and infestations (4.8%, 5.0%, 5.0%, 5.0%); PTs of RCTC grade 3 reported for >1 patient in any treatment group were neutropenia (180 mg: 5.0%, 240 mg: 5.0%), lymphopenia (240 mg: 5.0%, placebo 2.5%), and weight increased (240 mg: 5.0%).

Blood and lymphatic system disorders of maximum RCTC grade 4 were most frequently reported in the 240 mg group (120 mg: 4.8%, 180 mg: 5.0%, 240 mg: 7.5%, placebo: 2.5%); infections and infestations of RCTC grade 4 were only reported in the 240 mg group (5.0%); The only PT of RCTC grade 4 that was reported for >1 patient in any treatment group was neutropenia (120 mg: 4.8%, 180 mg: 5.0%, 240 mg: 7.5%).

AESIs were reported in the categories opportunistic infections (120 mg: 19.0%, 180 mg: 10.0%, 240 mg: 25.0%, placebo: 12.5%), severe infections (4.8%, 5.0%, 7.5%, 5.0%), and hepatic injury (placebo: 2.5%). AESIs on PT level reported for more than 1 patient in any treatment group were herpes zoster (120 mg: 4.8%, 180 mg: 5.0%, 240 mg: 12.5%, placebo: 7.5%), oral candidiasis (120 mg: 9.5%), gastroenteritis (240 mg: 5.0%), and septic shock (240 mg: 5.0%).

Serious adverse events (SAEs) were most frequently reported in the SOC infections and infestations (120 mg: 9.5%, 180 mg: 10.0%, 240 mg: 20.0%, placebo: 7.5%). At the PT level, SAEs reported for >1 patient in a treatment group were septic shock (240 mg: 5.0%) and neutropenia (120 mg: 4.8%, 180 mg: 10.0%, 240 mg: 2.5%). One death was reported in this trial: a 30-year-old female patient died of bacterial pneumonia, acute respiratory failure, and ventricular tachycardia, which were assessed by the investigator as not related to the study drug.

The evaluation of laboratory parameters revealed an imbalance between the treatment groups for neutrophils: the proportion of patients with a low neutrophil count on treatment was higher in the 240 mg group than in the other treatment groups.

### Conclusions

BI 655064 (at a dose of 120, 180, and 240 mg) added to standard of care therapy in patients with active lupus nephritis seemed to have no benefit over placebo in terms of CRR after 52 weeks of treatment. CRR was achieved by about 48% of patients in the placebo group, 38% in the 120 mg group, 45% in the 180 mg group, and 45% in the 240 mg group. The placebo response rate observed at Week 52 was higher than expected.

However, the analysis of confirmed CRR at 2 time points (Week 46 and Week 52) resulted in a reduced placebo response and a numerical benefit of the 180 mg dose over placebo, which was consistent with the improvements observed in total SLEDAI-SELENA score and its subscores.

While treatment with BI 655064 was safe and well tolerated at a dose of 120 and 180 mg, the highest BI 655064 dose (240 mg) was associated with higher frequencies of severe and serious infections, opportunistic infections, and neutropenia.

### Post Hoc Analysis Results and Discussion

### Efficacy

### B 655064 Effects on CRR and PRR

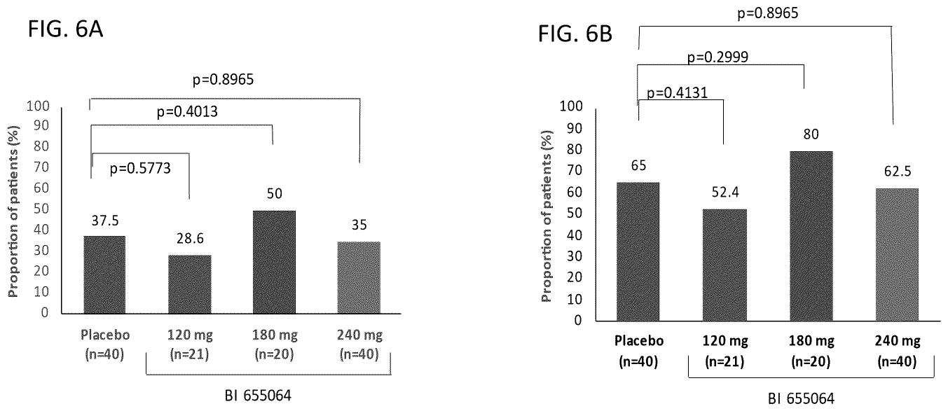

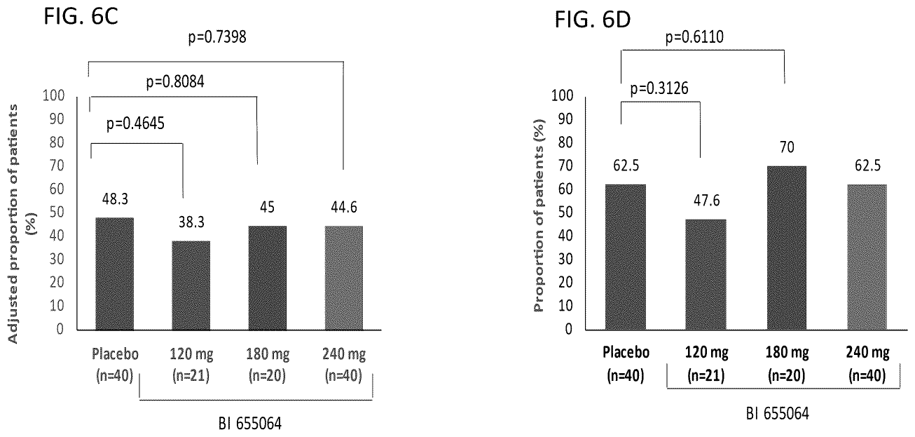

Based on UP (urine protein) from 24-hour collections: The proportion of patients with a CRR at Week 26 was higher in the BI 655064 180 mg dose group than in the placebo group (50.0% vs 37.5%; FIG. 6A). The BI 655064 180 mg group was the only treatment group with a higher proportion of patients achieving a CRR or PRR than the placebo group after both 26 and 52 weeks of treatment (FIGS. 6B and 6D). The adjusted proportion of patients who achieved a CRR at Week 52 was higher in the placebo group than in any of the BI 655064 groups (FIG. 6C).

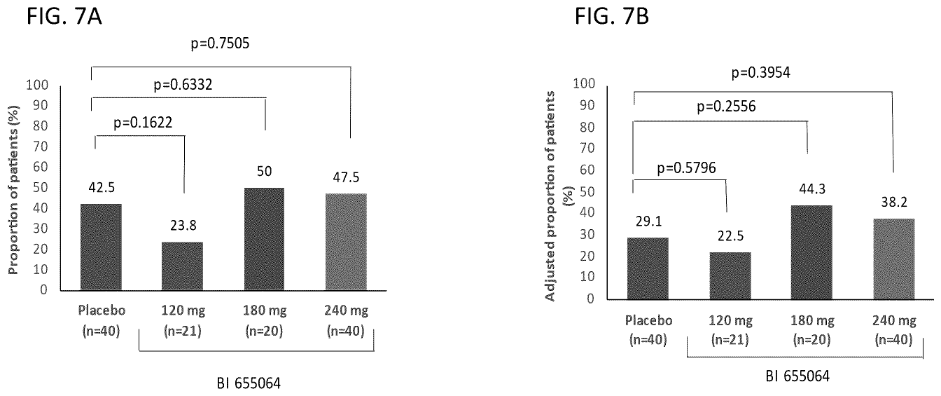

Based on UP/UC from spot urine: The proportion of patients who achieved a CRR at Week 52 was higher in the BI 655064 180 mg and 240 mg groups than in the placebo group (FIG. 7A). In a post hoc analysis of the confirmed CRR (cCRR) at Weeks 46 and 52, the adjusted proportion of patients achieving a cCRR was higher in the BI 655064 180 mg and 240 mg groups compared with the placebo group (44.3% and 38.2% vs 29.1%, respectively; FIG. 7B).

### Assessment of Further Endpoints

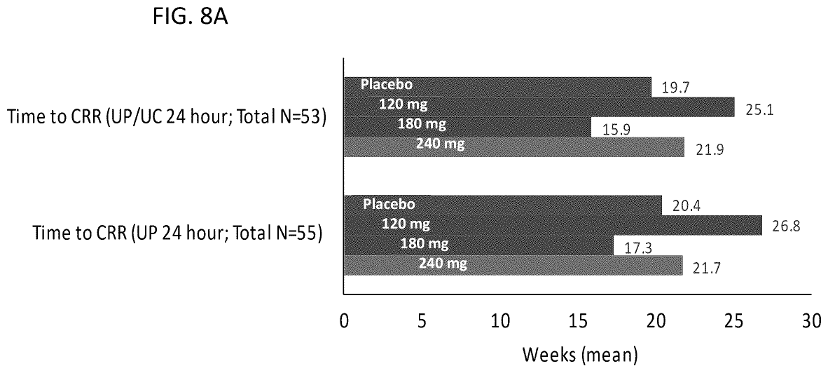

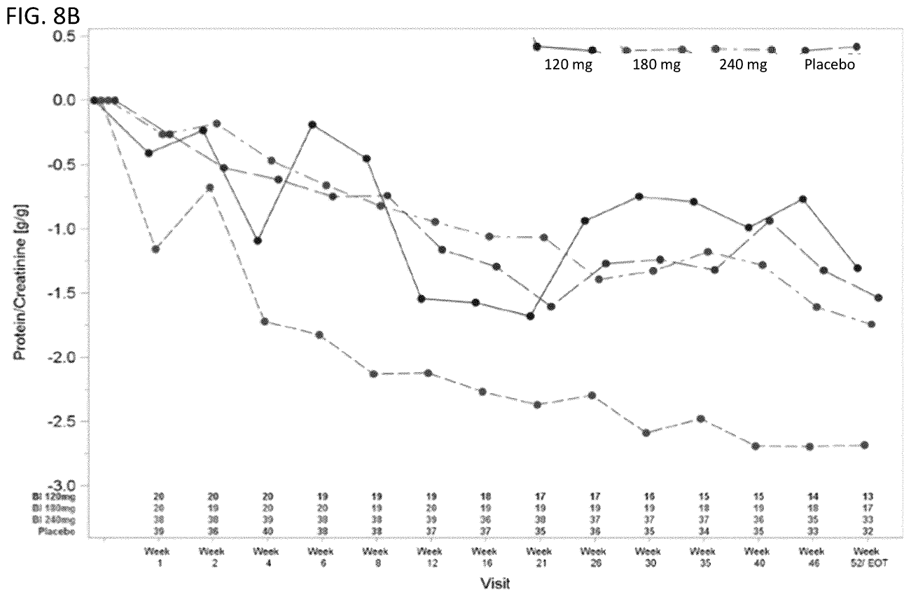

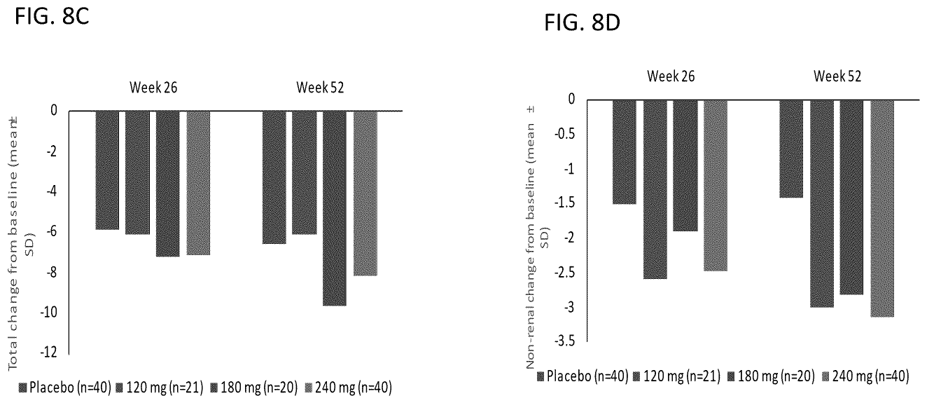

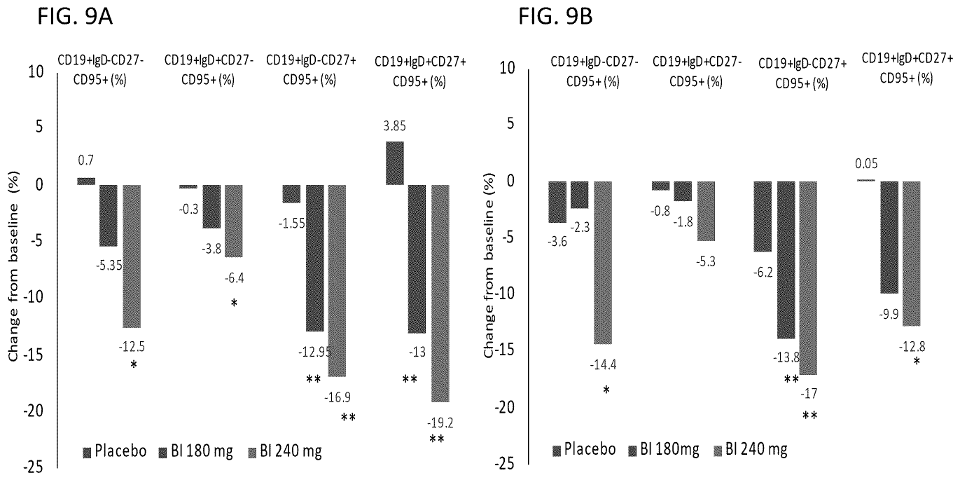

Of the patients who achieved a CRR at Week 52, patients in the BI 655064 180 mg group achieved a CRR in a shorter period of time than patients in the other treatment groups (FIG. 8A). An analysis of median change in UP/UC based on spot urine from baseline over time demonstrated that the BI 655064 180 mg group had a more substantial reduction in UP/UC over time than the other groups (FIG. 8B). The decrease from baseline in total Systemic Lupus Erythematosus Disease Activity Index (SLEDAI) was greater in the BI 655064 180 mg and 240 mg groups at Week 52 than in the other treatment groups (FIG. 8C). The decrease from baseline in non-renal SLEDAI mean scores was greater in the BI 655064 groups than in the placebo group (FIG. 8D). Significant decreases were observed in select activated B-cell subsets in the BI 655064 180 mg and 240 mg groups versus the placebo group at Weeks 12 and 26 post treatment (FIGS. 9A and 9B).

### Safety

The frequency of AEs was comparable across all treatment groups. A higher proportion of patients in the BI 655064 240 mg group experienced serious infections and neutropenia than placebo and other treatment groups; however, neutropenia was not associated with infections. Grade 4 infections were only reported in the BI 655064 240 mg group (two patients, 5.0%). No safety findings of concern were reported in the BI 655064 180 mg group.

### Discussion

The results of this trial suggest that the inhibition of CD40-CD40L shows some benefit in patients with LN, and demonstrate the feasibility of targeting CD40 without causing thromboembolic events. The trial did not meet the primary endpoint of CRR at Week 52 based on UP from 24-hour urine collection. The unexpectedly high placebo response prompted a post hoc analysis to evaluate cCRR. The BI 655064 180 mg and 240 mg groups had higher cCRR at Weeks 46 and 52 than the placebo group by 15.2% and 9.1%, respectively. The improvement in SLEDAI scores among patients treated with BI 655064 supports its potential application in the treatment of patients with active LN.

Significant decreases in select activated memory B-cell subsets were observed in the BI 655064 groups compared with the placebo group, which supports the clinical effects observed. BI 655064 180 mg was well-tolerated in comparison with the 240 mg group. The incidences of serious and severe infections and neutropenia were higher in patients in the 240 mg dose group than placebo and other treatment groups. The results from the post hoc analysis of cCRR offers evidence that BI 655064 180 mg could be effective in LN. Second year treatment of responders is ongoing.

The favourable effects of BI 655064 on SLEDAI scores in patients with LN, and its previously demonstrated efficacy in rheumatoid arthritis, indicate that BI 655064 may provide benefit in patients with non-renal SLE conditions and warrants further investigation.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

All patents and/or publications including journal articles cited in this disclosure are expressly incorporated herein by reference.

1. A method of treating lupus nephritis in a subject in need thereof, the method comprising administering to a subject with lupus nephritis class III, IV, or V a composition comprising an anti-CD40 antibody, wherein the anti-CD40 antibody comprises a heavy chain CDR1 sequence of SEQ ID NO: 11, a heavy chain CDR2 sequence of SEQ ID NO:15 and a heavy chain CDR3 sequence of SEQ ID NO:17; and wherein said antibody comprises a light chain CDR1 sequence of SEQ ID NO:21, a light chain CDR2 sequence of SEQ ID NO:23 and a light chain CDR3 sequence of SEQ ID NO:25,
wherein the composition comprises 120 mg, 180 mg or 240 mg of the anti-CD40 antibody;
wherein a single-dose administration of the composition comprising 120 mg of the anti-CD40 antibody results in Cₘₐₓ (ng mL⁻¹) of about 5160 to about 7210, AUC_{0-tz} (g·h mL⁻¹) of about 1110 to about 2010, or AUC_{0-inf} (g·h mL⁻¹) of about 1120 to about 2020; or
wherein a single-dose administration of the composition comprising 180 mg of the anti-CD40 antibody results in Cₘₐₓ (ng mL⁻¹) of about 8650 to about 16300, AUC_{0-tz} (g·h mL⁻¹) of about 2900 to about 6380, or AUC_{0-inf} (μg·h mL⁻¹) of about 2020 to about 2910; or
wherein a single-dose administration of the composition comprising 240 mg of the anti-CD40 antibody results in Cₘₐₓ (ng mL⁻¹) of about 15700 to about 21300, AUC_{0-tz} (μg h mL⁻¹) of about 5680 to about 7750, or AUC_{0-inf} (μg·h mL⁻¹) of about 5610 to about 7780; or
wherein a multiple-dose administration (q1w or once every week) of the composition comprising 240 mg of the anti-CD40 antibody results in C_{max,1} (μg mL⁻¹) of about 23 after the first dose or AUC_{τ, 1} (μg·h mL⁻¹) of about 2600 after the first dose; or
wherein a multiple-dose administration (q1w or once every week) of the composition comprising 240 mg of the anti-CD40 antibody results in C_{max,4} (μg mL⁻¹) of about 74 after the frouth dose, or C_{min, 4}(μg mL⁻¹) of about 49 after the fourth dose, or AUC_{τ, 4} (μg·h mL⁻¹) of about 10900 after the fourth dose.

2.-6. (canceled)

7. The method of claim 1, wherein the antibody comprises:
a heavy chain variable domain comprising SEQ ID NO:44 and a light chain variable domain comprising SEQ ID NO:43; or
a heavy chain variable domain comprising SEQ ID NO:53 and a light chain variable domain comprising SEQ ID NO:52; or
a heavy chain variable domain comprising SEQ ID NO:58 and a light chain variable domain comprising SEQ ID NO:56.

8.-9. (canceled)

10. A method of treating lupus nephritis in a subject in need thereof, the method comprising administering to a subject with lupus nephritis class III, IV, or V a composition comprising an anti-CD40 antibody, wherein the anti-CD40 antibody comprises a heavy chain CDR1 sequence of SEQ ID NO: 11, a heavy chain CDR2 sequence of SEQ ID NO:15 and a heavy chain CDR3 sequence of SEQ ID NO:17; and wherein said antibody comprises a light chain CDR1 sequence of SEQ ID NO:21, a light chain CDR2 sequence of SEQ ID NO:23 and a light chain CDR3 sequence of SEQ ID NO:25,
wherein the composition comprises 120 mg or 180 mg or 240 mg of the anti-CD40 antibody, and wherein the administration results in an improvement in total SLEDAI-SELENA score and its subscores in the subject as compared to placebo.

11. A method of determining the treatment efficacy of an anti-CD40 antibody in treating or preventing an autoimmune or inflammatory disease in a subject, the method comprising administering to the subject a composition comprising the anti-CD40 antibody, measuring the levels of an activated B-cell subset in the subject, wherein a decrease in the levels of the activated B-cell subset (when comparing the levels before and after the treatment) is indicative of efficacy, wherein the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺.

12. A method of decreasing the levels of an activated B-cell subset in a subject suffering from lupus nephritis, the method comprising administering to a subject with lupus nephritis class III, IV, or V a composition comprising an anti-CD40 antibody, wherein the anti-CD40 antibody comprises a heavy chain CDR1 sequence of SEQ ID NO: 11, a heavy chain CDR2 sequence of SEQ ID NO:15 and a heavy chain CDR3 sequence of SEQ ID NO:17; and wherein said antibody comprises a light chain CDR1 sequence of SEQ ID NO:21, a light chain CDR2 sequence of SEQ ID NO:23 and a light chain CDR3 sequence of SEQ ID NO:25, and wherein the activated B-cell subset is selected from the group consisting of CD19⁺IgD⁻CD27⁻CD95⁺, CD19+IgD+CD27−CD95⁺, CD19⁺IgD⁻CD27⁺CD95⁺ and CD19⁺IgD⁺CD27⁺CD95⁺.

13-15. (canceled)

16. The method of claim 1, wherein the composition comprises 120 mg of the anti-CD40 antibody.

17. The method of claim 1, wherein the composition comprises 180 mg of the anti-CD40 antibody.

18. The method of claim 1, wherein the composition comprises 240 mg of the anti-CD40 antibody.

19. The method of claim 10, wherein the antibody comprises a heavy chain variable domain comprising SEQ ID NO:53 and a light chain variable domain comprising SEQ ID NO:52.

20. The method of claim 10, wherein the composition comprises 120 mg of the anti-CD40 antibody.

21. The method of claim 10, wherein the composition comprises 180 mg of the anti-CD40 antibody.

22. The method of claim 10, wherein the composition comprises 240 mg of the anti-CD40 antibody.

23. The method of claim 12, wherein the antibody comprises a heavy chain variable domain comprising SEQ ID NO:53 and a light chain variable domain comprising SEQ ID NO:52.

24. The method of claim 12, wherein the composition comprises 120 mg of the anti-CD40 antibody.

25. The method of claim 12, wherein the composition comprises 180 mg of the anti-CD40 antibody.

26. The method of claim 12, wherein the composition comprises 240 mg of the anti-CD40 antibody.


SEQUENCE LISTING (78 sequences)

SEQ ID NO: 1 - protein, 118 aa, synthetic construct
  REGION 1..118: note = CD40 Murine Lead VH Sequence of 2H11
EVQLQQSGAELVRPGASVKLSCTASGFNIKDYYVHWVKQRPEKGLEWIGRIDPEDGDSKYAPKFQGKATMTADTSSNTAYLHLSSLTSEDTAVYYCTTSYYVGTYGYWGQGTTLTVSS

SEQ ID NO: 2 - protein, 118 aa, synthetic construct
  REGION 1..118: note = CD40 Murine Lead VH Sequence of 10F2
EVQLQQSGAELVRPGASVKLSCTASGFNIKDYYIHWVKQRPEKGLEWIGRIDPEDGDTKYDPKFQGKATMTADTSSNTAYLHLSSLTSEDTAVYYCTTSYYVGTYGYWGQGTTLTVSS

SEQ ID NO: 3 - protein, 118 aa, synthetic construct
  REGION 1..118: note = CD40 Murine Lead VH Sequence of 19B10
EVQLQQSGAELVRPGASVQLSCTASGFNIKDYYVHWVKQRPEKGLEWIGRIDPEDGDTKFAPKFQGKATMTADTSSNTVYLHLSSLTSEDTAVYYCTTSYYVGTYGYWGQGTTLTVSS

SEQ ID NO: 4 - protein, 119 aa, synthetic construct
  REGION 1..119: note = CD40 Murine Lead VH Sequence of 20E2
EVQLVESGGGLVKPGGSRKLSCAASGFTFSDYGMHWVRQAPEKGLEWVAYISSGNRIIYYADTVKGRFTISRDNAKNTLFLQMTSLRSEDTALYYCARQDGYRYAMDYWGQGTSVTVSS

SEQ ID NO: 5 - protein, 106 aa, synthetic construct
  REGION 1..106: note = CD40 Murine Lead VK Sequence of 2H11
QIVLTQSPAIMSASPGEKVTITCSASSSVSYMLWFQQKPGTSPKLWIYSTSNLASGVPARFGGSGSGTSYSLTISRMEAEDAATYYCQQRTFYPYTFGGGTKLEIK

SEQ ID NO: 6 - protein, 106 aa, synthetic construct
  REGION 1..106: note = CD40 Murine Lead VK Sequence of 10F2
QIVLTQSPTIMSASPGEKVIITCSATSSVSYILWFQQKPGTSPKLWIYSTSNLASGVPARFSGSGSGASYSLTISRMEAEDAATYYCQQRTFYPYTFGGGTKLEIK

SEQ ID NO: 7 - protein, 106 aa, synthetic construct
  REGION 1..106: note = CD40 Murine Lead VK Sequence of 19B10
QIVLTQSPAIMSASPGEKVTITCSASSSVSYMLWFQQKPGTSPKLWIYSTSNLASGVPARFSGSGSGTSYSLTISRMEAEDAATYYCQQRTFYPYTFGGGTKLEIK

SEQ ID NO: 8 - protein, 113 aa, synthetic construct
  REGION 1..113: note = CD40 Murine Lead VK Sequence of 20E2
DIVMTQSPSSLTVTAGEKVTMSCKSSQSLLNSGNQKNYLTWHQQKPGQPPKLLIYWTSTRESGVPDRFTGSGSGTDFTLTISNLQAEDLAVYYCQNDYTYPLTFGAGTKLELK

SEQ ID NO: 9 - protein, 10 aa, synthetic construct
  REGION 1..10: note = CDR1 Heavy Chain of 2H11 and 19B10
GFNIKDYYVH

SEQ ID NO: 10 - protein, 10 aa, synthetic construct
  REGION 1..10: note = CDR1 Heavy Chain of 10F2
GFNIKDYYIH

SEQ ID NO: 11 - protein, 10 aa, synthetic construct
  REGION 1..10: note = CDR1 Heavy Chain of 20E2
GFTFSDYGMH

SEQ ID NO: 12 - protein, 17 aa, synthetic construct
  REGION 1..17: note = CDR2 Heavy Chain of 2H11
RIDPEDGDSKYAPKFQG

SEQ ID NO: 13 - protein, 17 aa, synthetic construct
  REGION 1..17: note = CDR2 Heavy Chain of 10F2
RIDPEDGDTKYDPKFQG

SEQ ID NO: 14 - protein, 17 aa, synthetic construct
  REGION 1..17: note = CDR2 Heavy Chain of 19B10
RIDPEDGDTKFAPKFQG

SEQ ID NO: 15 - protein, 17 aa, synthetic construct
  REGION 1..17: note = CDR2 Heavy Chain of 20E2
YISSGNRIIYYADTVKG

SEQ ID NO: 16 - protein, 9 aa, synthetic construct
  REGION 1..9: note = CDR3 Heavy Chain of 2H11, 10F2 and 19B10
SYYVGTYGY

SEQ ID NO: 17 - protein, 10 aa, synthetic construct
  REGION 1..10: note = CDR3 Heavy Chain of 20E2
QDGYRYAMDY

SEQ ID NO: 18 - protein, 10 aa, synthetic construct
  REGION 1..10: note = CDR1 Light Chain of 2H11
SASSSVSYML

SEQ ID NO: 19 - protein, 10 aa, synthetic construct
  REGION 1..10: note = CDR1 Light Chain of 10F2
SATSSVSYIL

SEQ ID NO: 20 - protein, 10 aa, synthetic construct
  REGION 1..10: note = CDR1 Light Chain of 19B10
SASSSVSYML

SEQ ID NO: 21 - protein, 17 aa, synthetic construct
  REGION 1..17: note = CDR1 Light Chain of 20E2
KSSQSLLNSGNQKNYLT

SEQ ID NO: 22 - protein, 7 aa, synthetic construct
  REGION 1..7: note = CDR2 Light Chain of 2H11, 10F2 and 19B10
STSNLAS

SEQ ID NO: 23 - protein, 7 aa, synthetic construct
  REGION 1..7: note = CDR2 Light Chain of 20E2
WTSTRES

SEQ ID NO: 24 - protein, 9 aa, synthetic construct
  REGION 1..9: note = CDR3 Light Chain of 2H11, 10F2, and 19B10
QQRTFYPYT

SEQ ID NO: 25 - protein, 9 aa, synthetic construct
  REGION 1..9: note = CDR3 Light Chain of 20E2
QNDYTYPLT

SEQ ID NO: 26 - protein, 220 aa, synthetic construct
  REGION 1..220: note = Light Chain of Antibody A
DIVMTQSPDSLAVSLGERATMSCKSSQSLLNSGNQKNYLTWHQQKPGQPPKLLIYWTSTRESGVPDRFSGSGSGTDFTLTISSLQAEDVAVYYCQNDYTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 449 aa, synthetic construct
  REGION 1..449: note = Heavy Chain IgG1K0 of Antibody A
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 449 aa, synthetic construct
  REGION 1..449: note = Heavy Chain IgG1 of Antibody A
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 446 aa, synthetic construct
  REGION 1..446: note = Heavy Chain IgG4DM of Antibody A
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 449 aa, synthetic construct
  REGION 1..449: note = Heavy Chain IgG1K0b of Antibody A
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 220 aa, synthetic construct
  REGION 1..220: note = Light Chain of Antibody B
DIVMTQSPDSLAVSLGEKVTINCKSSQSLLNSGNQKNYLTWHQQKPGQPPKLLIYWTSTRESGVPDRFSGSGSGTDFTLTISSLQAEDVAVYYCQNDYTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 449 aa, synthetic construct
  REGION 1..449: note = Heavy Chain IgG1K0 of Antibody B
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 449 aa, synthetic construct
  REGION 1..449: note = Heavy Chain IgG1 of Antibody B
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 446 aa, synthetic construct
  REGION 1..446: note = Heavy Chain IgG4DM of Antibody B
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 449 aa, synthetic construct
  REGION 1..449: note = Heavy Chain IgG1K0b of Antibody B
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 213 aa, synthetic construct
  REGION 1..213: note = Light Chain of Antibody C
DIQMTQSPSSLSASVGDRVTITCSASSSVSYMLWFQQKPGKAPKLLIYSTSNLASGVPSRFSGSGSGTDFTLTISSLQPEDFATYYCQQRTFYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 448 aa, synthetic construct
  REGION 1..448: note = Heavy Chain IgG1K0 of Antibody C
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 448 aa, synthetic construct
  REGION 1..448: note = Heavy Chain IgG1 of Antibody C
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 445 aa, synthetic construct
  REGION 1..445: note = Heavy Chain IgG4DM of Antibody C
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 448 aa, synthetic construct
  REGION 1..448: note = Heavy Chain IgG1K0b of Antibody C
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 113 aa, synthetic construct
  REGION 1..113: note = Variable Light Chain
DIVMTQSPDSLAVSLGERVTMSCKSSQSLLNSGNQKNYLTWHQQKPGQPPKLLIYWTSTRESGVPDRFSGSGSGTDFTLTISSLQAEDVAVYYCQNDYTYPLTFGGGTKVEIK

SEQ ID NO: 42 - protein, 119 aa, synthetic construct
  REGION 1..119: note = Variable Heavy Chain
EVQLVKSGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWGQGTLVTVSS

SEQ ID NO: 43 - protein, 113 aa, synthetic construct
  REGION 1..113: note = Variable Light Chain in Antibody A
DIVMTQSPDSLAVSLGERATMSCKSSQSLLNSGNQKNYLTWHQQKPGQPPKLLIYWTSTRESGVPDRFSGSGSGTDFTLTISSLQAEDVAVYYCQNDYTYPLTFGGGTKVEIK

SEQ ID NO: 44 - protein, 119 aa, synthetic construct
  REGION 1..119: note = Variable Heavy Chain Antibody A
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSS

SEQ ID NO: 45 - protein, 113 aa, synthetic construct
  REGION 1..113: note = Variable Light Chain
DIVMTQSPDSLAVSLGEKVTMNCKSSQSLLNSGNQKNYLTWHQQKPGQPPKLLIYWTSTRESGVPDRFSGSGSGTDFTLTISSLQAEDVAVYYCQNDYTYPLTFGAGTKVEIK

SEQ ID NO: 46 - protein, 119 aa, synthetic construct
  REGION 1..119: note = Variable Heavy Chain
EVQLVESGGGLVKPGGSRRLSCAASGFTFSDYGMHWVRQAPGKGLEWVAYISSGNRIIYYADTVKGRFTISRDNAKNSLYLQMNSLRAEDTALYYCARQDGYRYAMDYWGQGTLVTVSS

SEQ ID NO: 47 - protein, 113 aa, synthetic construct
  REGION 1..113: note = Variable Light Chain
DIVMTQSPDSLAVSLGERVTMNCKSSQSLLNSGNQKNYLTWHQQKPGQPPKLLIYWTSTRESGVPDRFSGSGSGTDFTLTISSLQAEDVAVYYCQNDYTYPLTFGGGTKVEIK

SEQ ID NO: 48 - protein, 119 aa, synthetic construct
  REGION 1..119: note = Variable Heavy Chain
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWGQGTLVTVSS

SEQ ID NO: 49 - protein, 113 aa, synthetic construct
  REGION 1..113: note = Variable Light Chain
DIVMTQSPDSLAVSLGERVTMNCKSSQSLLNSGNQKNYLTWHQQKPGQPPKLLIYWTSTRESGVPDRFSGSGSGTDFTLTISSLQAEDVAVYYCQNDYTYPLTFGAGTKVEIK

SEQ ID NO: 50 - protein, 119 aa, synthetic construct
  REGION 1..119: note = Variable Light Chain
EVQLVESGGGLVKPGGSRRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSS

SEQ ID NO: 51 - protein, 113 aa, synthetic construct
  REGION 1..113: note = Variable Light Chain
DIVMTQSPDSLAVSLGEKVTMNCKSSQSLLNSGNQKNYLTWHQQKPGQPPKLLIYWTSTRESGVPDRFSGSGSGTDFTLTISSLQAEDLAVYYCQNDYTYPLTFGAGTKVEIK

SEQ ID NO: 52 - protein, 113 aa, synthetic construct
  REGION 1..113: note = Variable Light Chain in Antibody B
DIVMTQSPDSLAVSLGEKVTINCKSSQSLLNSGNQKNYLTWHQQKPGQPPKLLIYWTSTRESGVPDRFSGSGSGTDFTLTISSLQAEDVAVYYCQNDYTYPLTFGGGTKVEIK

SEQ ID NO: 53 - protein, 119 aa, synthetic construct
  REGION 1..119: note = Variable Heavy Chain
EVQLVESGGGLVKPGGSLRLSCAASGFTFSDYGMHWVRQAPGKGLEWVAYISSGNRIIYYADTVKGRFTISRDNAKNSLYLQMNSLRAEDTAVYYCARQDGYRYAMDYWGQGTLVTVSS

SEQ ID NO: 54 - protein, 106 aa, synthetic construct
  REGION 1..106: note = Variable Light Chain
QIQMTQSPSSLSASVGDRVTITCSASSSVSYMLWFQQKPGKAPKLWIYSTSNLASGVPARFSGSGSGTDFTLTISSLQPEDFATYYCQQRTFYPYTFGGGTKVEIK

SEQ ID NO: 55 - protein, 106 aa, synthetic construct
  REGION 1..106: note = Variable Light Chain
DIQMTQSPSSLSASVGDRVTITCSASSSVSYMLWFQQKPGKAPKLLIYSTSNLASGVPARFSGSGSGTDFTLTISSLQPEDFATYYCQQRTFYPYTFGGGTKVEIK

SEQ ID NO: 56 - protein, 106 aa, synthetic construct
  REGION 1..106: note = Variable Light Chain
DIQMTQSPSSLSASVGDRVTITCSASSSVSYMLWFQQKPGKAPKLLIYSTSNLASGVPSRFSGSGSGTDFTLTISSLQPEDFATYYCQQRTFYPYTFGGGTKVEIK

SEQ ID NO: 57 - protein, 118 aa, synthetic construct
  REGION 1..118: note = Variable Heavy Chain
QVQLVQSGAEVKKPGASVKVSCTASGFNITDYYVHWVKQRPGQGLEWMGRIDPEDGDSKYAPKFQGKATMTADTSTSTVYMELSSLRSEDTAVYYCTTSYYVGTYGYWGQGTLVTVSS

SEQ ID NO: 58 - protein, 118 aa, synthetic construct
  REGION 1..118: note = Variable Heavy Chain in Antibody C
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSS

SEQ ID NO: 59 - protein, 118 aa, synthetic construct
  REGION 1..118: note = Variable Heavy Chain
QVQLVQSGAEVKKPGASVKVSCTASGFNITDYYVHWVKQRPGQGLEWMGRIDPEDGDSKYAPKFQGKVTMTADTSTSTVYMELSSLRSEDTAVYYCTTSYYVGTYGYWGQGTLVTVSS

SEQ ID NO: 60 - protein, 118 aa, synthetic construct
  REGION 1..118: note = Variable Heavy Chain
QVQLVQSGAEVKKPGASVKVSCTASGFNIKDYYVHWVKQAPGQGLEWIGRIDPEDGDSKYAPKFQGKATMTADTSTSTVYMELSSLRSEDTAVYYCTTSYYVGTYGYWGQGTLVTVSS

SEQ ID NO: 61 - protein, 118 aa, synthetic construct
  REGION 1..118: note = Variable Heavy Chain
QVQLVQSGAEVKKPGASVKVSCTASGFNITDYYVHWVKQAPGQGLEWMGRIDPEDGDSKYAPKFQGKATMTADTSTSTVYMELSSLRSEDTAVYYCTTSYYVGTYGYWGQGTLVTVSS

SEQ ID NO: 62 - protein, 118 aa, synthetic construct
  REGION 1..118: note = Variable Heavy Sequence Version 1 from Antibody 19B10-Hum
QVQLVQSGAEVKKPGASVKVSCTASGFNITDYYVHWVKQRPGQGLEWMGRIDPEDGDTKFAPKFQGKATMTADTSTSTVYMELSSLRSEDTAVYYCTTSYYVGTYGYWGQGTLVTVSS

SEQ ID NO: 63 - protein, 118 aa, synthetic construct
  REGION 1..118: note = Variable Heavy Sequence Version 2 from Antibody 19B10-Hum
QVQLVQSGAEVKKPGASVKVSCTASGFNITDYYVHWVKQRPGQGLEWMGRIDPEDGDTKFAPKFQGKVTMTADTSTSTVYMELSSLRSEDTAVYYCTTSYYVGTYGYWGQGTLVTVSS

SEQ ID NO: 64 - protein, 118 aa, synthetic construct
  REGION 1..118: note = Variable Heavy Sequence Version 3 from Antibody 19B10-Hum
QVQLVQSGAEVKKPGASVKVSCTASGFNIKDYYVHWVKQAPGQGLEWIGRIDPEDGDTKFAPKFQGKATMTADTSTSTVYMELSSLRSEDTAVYYCTTSYYVGTYGYWGQGTLVTVSS

SEQ ID NO: 65 - protein, 118 aa, synthetic construct
  REGION 1..118: note = Variable Heavy Sequence Version 4 from Antibody 19B10-Hum
QVQLVQSGAEVKKPGASVKVSCTASGFNIKDYYVHWVKQAPGQGLEWMGRIDPEDGDTKFAPKFQGKATMTADTSTSTVYMELSSLRSEDTAVYYCTTSYYVGTYGYWGQGTLVTVSS

SEQ ID NO: 66 - protein, 118 aa, synthetic construct
  REGION 1..118: note = Variable Heavy Sequence Version 5 from Antibody 19B10-Hum
QVQLVQSGAEVKKPGASVKVSCTASGFNITDYYVHWVKQAPGQGLEWMGRIDPEDGDTKFAPKFQGKATMTADTSTSTVYMELSSLRSEDTAVYYCTTSYYVGTYGYWGQGTLVTVSS

SEQ ID NO: 67 - protein, 118 aa, synthetic construct
  REGION 1..118: note = Variable Heavy Sequence Version 1 from Antibody 10F2Hum
EVQLVQSGAEVKKPGATVKISCKVSGFNIKDYYIHWVKQRPGKGLEWMGRIDPEDGDTKYDPKFQGRVTMTADTSTDTAYMELSSLRSEDTAVYYCTTSYYVGTYGYWGQGTTVTVSS

SEQ ID NO: 68 - protein, 118 aa, synthetic construct
  REGION 1..118: note = Variable Heavy Sequence Version 2 from Antibody 10F2Hum
EVQLVQSGAEVKKPGATVKISCTVSGFNIKDYYIHWVKQRPGKGLEWMGRIDPEDGDTKYDPKFQGRVTMTADTSTDTAYMELSSLRSEDTAVYYCTTSYYVGTYGYWGQGTTVTVSS

SEQ ID NO: 69 - protein, 118 aa, synthetic construct
  REGION 1..118: note = Variable Heavy Sequence Version 3 from Antibody 10F2Hum
EVQLVQSGAEVKKPGATVKISCTVSGFNIKDYYIHWVKQRPGKGLEWMGRIDPEDGDTKYDPKFQGKVTMTADTSTDTAYMELSSLRSEDTAVYYCTTSYYVGTYGYWGQGTTVTVSS

SEQ ID NO: 70 - protein, 118 aa, synthetic construct
  REGION 1..118: note = Variable Heavy Sequence Version 4 from Antibody 10F2Hum
EVQLVQSGAEVKKPGATVKISCTVSGFNIKDYYIHWVKQAPGKGLEWMGRIDPEDGDTKYDPKFQGKATMTADTSTDTAYMELSSLRSEDTAVYYCTTSYYVGTYGYWGQGTTVTVSS

SEQ ID NO: 71 - protein, 118 aa, synthetic construct
  REGION 1..118: note = Variable Heavy Sequence Version 5 from Antibody 10F2Hum
EVQLVQSGAEVKKPGATVKISCTVSGFNIKDYYIHWVKQRPGKGLEWMGRIDPEDGDTKYDPKFQGKATMTADTSTDTAYMELSSLRSEDTAVYYCTTSYYVGTYGYWGQGTTVTVSS

SEQ ID NO: 72 - protein, 118 aa, synthetic construct
  REGION 1..118: note = Variable Heavy Sequence Version 6 from Antibody 10F2Hum
EVQLVQSGAEVKKPGATVKISCTVSGFNIKDYYIHWVKQAPGKGLEWIGRIDPEDGDTKYDPKFQGKATMTADTSTDTAYMELSSLRSEDTAVYYCTTSYYVGTYGYWGQGTTVTVSS

SEQ ID NO: 73 - protein, 118 aa, synthetic construct
  REGION 1..118: note = Variable Heavy Sequence Version 7 from Antibody 10F2Hum
EVQLVQSGAEVKKPGATVKISCKVSGFNIKDYYIHWVQQAPGKGLEWMGRIDPEDGDTKYDPKFQGRVTMTADTSTDTAYMELSSLRSEDTAVYYCTTSYYVGTYGYWGQGTTVTVSS

SEQ ID NO: 74 - protein, 106 aa, synthetic construct
  REGION 1..106: note = Variable Light Sequence Version 1 from Antibody 10F2Hum
DIQMTQSPSSLSASVGDRVTITCSATSSVSYILWFQQKPGKAPKLLIYSTSNLASGVPSRFSGSGSGTDFTLTISSLQPEDFATYYCQQRTFYPYTFGGGTKVEIK

SEQ ID NO: 75 - protein, 106 aa, synthetic construct
  REGION 1..106: note = Variable Light Sequence Version 2 from Antibody 10F2Hum
DIQMTQSPSSLSASVGDRVTITCSATSSVSYILWFQQKPGKAPKLLIYSTSNLASGVPARFSGSGSGTDFTLTISSLQPEDFATYYCQQRTFYPYTFGGGTKVEIK

SEQ ID NO: 76 - protein, 106 aa, synthetic construct
  REGION 1..106: note = Variable Light Sequence Version 3 from Antibody 10F2Hum
QIQMTQSPSSLSASVGDRVTITCSATSSVSYILWFQQKPGKAPKLWIYSTSNLASGVPARFSGSGSGTDFTLTISSLQPEDFATYYCQQRTFYPYTFGGGTKVEIK

SEQ ID NO: 77 - protein, 11 aa, synthetic construct
  REGION 1..11: note = Heavy Chain CDR3 for 2H11, 10F2 and 19B10
TTSYYVGTYGY

SEQ ID NO: 78 - protein, 12 aa, synthetic construct
  REGION 1..12: note = Heavy Chain CDR3 for 20E2
ARQDGYRYAMDY